(19) United States
(12) Patent Application Publication
(10) Pub. No.: US 2022/0389066 A1
(43) Pub. Date: Dec. 8, 2022

(54) POLYPEPTIDES

(71) Applicant: Affibody AB, Solna (SE)

(72) Inventor: Elisabet Wahlberg, Sigtuna (SE)

(21) Appl. No.: 17/771,307

(22) PCT Filed: Nov. 5, 2020
(86) PCT No.: PCT/EP2020/081120

§ 371 Date: Apr. 22, 2022

(30) Foreign Application Priority Data
Nov. 5, 2019 (EP) 19207172.8

(51) Int. Cl. C07K 14/435; C07K 19/00

(52) U.S. Cl. CPC C07K 14/435; C07K 19/00

(57) ABSTRACT

The present disclosure relates to a class of engineered polypeptides having a binding affinity for high mobility group box 1 protein (HMGB1), and provides an HMGB1 binding polypeptide comprising the sequence EX₂X₃X₄AX₆X₇EIX₁₀ X₁₁LPNLX₁₆X₁₇X₁₈QX₂₀X₂₁AFIYX₂₆LED or a sequence having at least 93% identity thereto. The present disclosure also relates to the use of such an HMGB1 binding polypeptide as a therapeutic, prognostic and/or diagnostic agent.

### FIELD OF THE INVENTION

The present disclosure relates to a class of engineered polypeptides having a binding affinity for the protein High Mobility Group Box 1 (in the following referred to as HMGB1). The present disclosure also relates to the use of such an HMGB1 binding polypeptide as a therapeutic, prognostic and/or diagnostic agent.

### BACKGROUND

High Mobility Group Box 1 (HMGB1) protein is a non-histone protein that has key roles both inside and outside the cell. Intracellularly, HMGB1 binds DNA and participates in a number of DNA-dependent processes, including transcription, replication, DNA repair and nucleosome assembly, serving as an architectural element. During activation and cell death, HMGB1 can translocate from the nucleus to the extracellular space. Extracellularly, HMGB1 serves as an alarmin to mediate and drive the pathogenesis of inflammatory and autoimmune disease.

HMGB1 induces inflammation by first recruiting inflammatory cells and then by activating these to release pro-inflammatory mediators. Two major pathways of HMGB1 release occur during inflammation: 1) active secretion by stressed or activated cells (chronic indications) and 2) passive release from damaged or dying cells (acute indications). These pathways are differentiated on the basis of molecular mechanisms, release kinetics, and down-stream signaling responses. Active secretion of HMGB1, initiated by cellular signal transduction through plasma membrane receptor interaction with extracellular products, occurs more slowly, whereas passive release, initiated by damage of cellular integrity in acute indications, is nearly instantaneous. Intracellular HMGB1 is retained by chromatin during apoptotic cell death and is not released unless apoptotic bodies undergo secondary necrosis. Extracellular HMGB1 and the C-X-C motif chemokine ligand 12, CXCL12, form a heterocomplex, initiating recruitment of inflammatory cells to damaged tissues, via binding to the C-X-C chemokine receptor type 4, CXCR4. Furthermore, HMGB1 mediates inflammatory responses via interactions with multiple well-known innate immunity receptors including toll-like receptor 4 (TLR4) and the receptor for advanced glycated end products (RAGE). In addition to these direct pro-inflammatory effects, HMGB1 also interacts with an ionotropic glutamate receptor, the N-methyl-D-aspartate (NMDA) receptor, to enhance glutamate activity and cytotoxicity (reviewed in Andersson and Tracey (2011) Annu Rev Immunol 29:139-62).

HMGB1 is a 25 kDa protein of 215 amino acids, whose primary sequence is more than 98% identical in all mammals. Structurally, HMGB1 is composed of three domains: two positively charged, proximal DNA-binding domains (A-box and B-box) and a negatively charged carboxyl terminus. The A-box and the B-box have distinct biological activities. The B-box recapitulates the inflammatory activities of the full-length protein, whereas the A-box antagonizes them. Three cysteines are encoded within the molecule: two cysteines in the A-box (C23 and C45) and one cysteine in the B-box (C106). C23 and C45 can form an intermolecular disulfide bond, whereas C106 is unpaired. The extracellular activity of HMGB1 depends on the redox states of the cysteine residues. Reduced HMGB1 (all-thiol-HMGB1) functions as a chemoattractant by interaction with CXCL12 and CXCR4 in a synergistic manner. Disulfide-HMGB1 (oxidized A-box, reduced B-box) is essential for the interaction with TLR4 to induce leukocyte activation and release of pro-inflammatory cytokines. Reactive oxygen species produced by leukocytes induce terminal oxidation of HMGB1, which abrogates both the chemoattractant and cytokine-inducing properties of HMGB1.

Infection, necrosis and apoptosis can all lead to elevated HMGB1 levels. At low elevated levels, HMGB1 mediates sickness behavior and antibacterial activities, which contribute to inflammatory responses that can resolve. The release of larger amounts of HMGB1, however, is associated with the development of epithelial barrier failure, organ dysfunction, and even death.

Extensive preclinical evidence suggests that blocking HMGB1 could be beneficial in a range of inflammatory diseases. Administration of agents that inhibit HMGB1 activity specifically (such as antibodies, fragments of HMGB1, soluble receptors and small molecule drugs) to animals with ischemia and inflammatory diseases interrupts the progression of tissue injury and suppresses inflammatory responses. 2G7 is a monoclonal antibody that binds to an epitope within the A-box of HMGB1. Therapeutic effects of 2G7 have been demonstrated in experimental models of sepsis, pancreatic islet graft transplantation, arthritis and drug-induced liver injury ((Qin et al (2006), J Exp Med 203(7):1637-42, Schierbeck et al (2011), Mol Med 17(9-10):1039-44, Lundbäck et al (2016), Hepatology 64(5):1699-1710). DPH1.1 is a monoclonal antibody that binds to an epitope at the end of the B box. DPH1.1 was shown to block HMGB1-elicited cell migration of 3T3 fibroblasts in vitro and leucocyte recruitment in vivo, and therapeutic effects have been demonstrated in experimental models of hepatitis B, diet-induced atherosclerosis, brain injury and mesothelioma (WO2012/136250). Small molecule inhibitors of HMGB1 release include ethyl pyruvate and glycyrrhizin (reviewed in Venereau et al (2016) Pharm Res 111:534-544).

The indication space for HMGB1 can be divided into acute and chronic inflammation. Acute conditions include for example cerebral ischemia, endotoxemia, ischemia-reperfusion injury, pancreatitis, sepsis, stroke, systemic inflammatory response syndrome (SIRS), transplantation and acute acetaminophen/paracetamol intoxication. Chronic indications include for example arthritis, vasculitis, myositis, and systemic lupus erythematosus (SLE).

Despite their proven efficacy in various preclinical models, no HMGB1 targeting antibody has yet been tested in clinical trials. Thus, there is a continuing need for the development of new modes of treatment.

### SUMMARY

It is an object of the present disclosure to provide new HMGB1 binding agents, which could for example be used for therapeutic, prognostic and diagnostic applications.

It is an object of the present disclosure to provide a new multispecific agent, such as a bispecific agent, with separate binding specificities for different parts of the HMGB1 protein, such as at least one part with affinity for the A-box of HMGB1 and another part with affinity for the B-box of HMGB1.

It is an object of the present disclosure to provide a new multispecific agent, such as a bispecific agent, which has affinity for HMGB1 and at least one additional antigen or target.

It is an object of the present disclosure to provide a molecule allowing for efficient therapy of for example various forms of inflammatory disease, while alleviating the abovementioned and other drawbacks of current therapies.

It is an object of the present disclosure to provide a molecule suitable for prognostic and diagnostic applications, for example prognostic and diagnostic application in relation to various forms of cancer and infectious disease.

These and other objects, which are evident to the skilled person from the present disclosure, are met by the different aspects of the invention as claimed in the appended claims and as generally disclosed herein.

Thus, in the first aspect of the disclosure, there is provided an HMGB1 binding polypeptide, comprising an HMGB1 binding motif BM, which motif consists of an amino acid sequence selected from:

i)

(SEQ ID NO: 56)
EX₂X₃X₄AX₆X₇EIX₁₀ X₁₁LPNLX₁₆X₁₇X₁₈QX₂₀ X₂₁AFIYX₂₆LED

wherein, independently from each other,
X₂ is selected from A, D, S and T;
X₃ is selected from E, R and W;
X₄ is selected from A, D and Q;
X₆ is selected from F and M;
X₇ is selected from E, H, W and Y;
X₁₀ is selected from I and L;
X₁₁ is selected from A and W;
X₁₆ is selected from N and T;
X₁₇ is selected from A, D, N and W;
X₁₈ is selected from A, E, Q, R, S, T and Y;
X₂₀ is selected from A and Q;
X₂₁ is selected from K, L and R; and
X₂₆ is selected from K and S;
   and
   ii) an amino acid sequence which has at least 93% identity to the sequence defined in i).

In another embodiment, there is provided an HMGB1 binding polypeptide wherein in sequence i)
X₂ is selected from A, D, S and T;
X₃ is selected from E, R and W;
X₄ is selected from D and Q;
X₆ is selected from F and M;
X₇ is selected from E, H, W and Y;
X₁₀ is selected from I and L;
X₁₁ is W;
X₁₆ is selected from N and T;
X₁₇ is selected from D, N and W;
X₁₈ is selected from A, E, Q, R, T and Y;
X₂₀ is Q;
X₂₁ is selected from K, L and R; and
X₂₆ is selected from K and S.

In another embodiment, there is provided an HMGB1 binding polypeptide, wherein in sequence i)
X₂ is selected from A, D, S and T;
X₃ is selected from E, R and W;
X₄ is selected from D and Q;
X₆ is selected from F and M;
X₇ is selected from E, H, W and Y;
X₁₀ is selected from I and L;
X₁₁ is W;
X₁₆ is selected from N and T;
X₁₇ is selected from D, N and W;
X₁₈ is selected from A, E, Q, R and T;
X₂₀ is Q;
X₂₁ is selected from K, L and R; and
X₂₆ is selected from K and S.

In yet another embodiment, there is provided an HMGB1 binding polypeptide, wherein in sequence i)
X₂ is selected from A and D;
X₃ is selected from E and W;
X₄ is selected from D and Q;
X₆ is selected from F and M;
X₇ is selected from E, H and W;
X₁₀ is selected from I and L;
X₁₁ is W;
X₁₆ is selected from N and T;
X₁₇ is selected from D, N and W;
X₁₈ is selected from A, E, and T;
X₂₀ is Q;
X₂₁ is selected from K, L and R; and
X₂₆ is selected from K and S.

In yet another embodiment, there is provided an HMGB1 binding polypeptide, wherein in sequence i)
X₂ is selected from A, D, S and T;
X₃ is selected from R and W;
X₄ is D;
X₆ is F;
X₇ is selected from E, Y and W;
X₁₀ is I;
X₁₁ is W;
X₁₆ is selected from N and T;
X₁₇ is D;
X₁₈ is selected from A, Q and R;
X₂₀ is Q;
X₂₁ is R; and
X₂₆ is selected from K and S.

As used herein, “Xₙ” and “Xₘ” are used to indicate amino acids in positions n and m in the sequence i) as defined above, wherein n and m are integers which indicate the position of an amino acid within said sequence as counted from the N-terminal end of said sequence. For example, X₃ and X₇ indicate the amino acid in position three and seven, respectively, from the N-terminal end of sequence i).

In embodiments according to the first aspect, there are provided polypeptides wherein Xₙ in sequence i) is independently selected from a group of possible residues according to Table 1. The skilled person will appreciate that Xₙ may be selected from any one of the listed groups of possible residues and that this selection is independent from the selection of amino acids in Xₘ, wherein n≠m. Thus, any of the listed possible residues in position Xₙ in Table 1 may be independently combined with any of the listed possible residues any other variable position in Table 1.

The skilled person will appreciate that Table 1 is to be read as follows: In one embodiment according to the first aspect, there is provided a polypeptide wherein amino acid residue “Xₙ” in sequence i) is selected from “Possible residues”. Thus, Table 1 discloses several specific and individualized embodiments of the first aspect of the present disclosure. For example, in one embodiment according to the first aspect, there is provided a polypeptide wherein X₄ in sequence i) is selected from A, D and Q, and in another embodiment according to the first aspect, there is provided a polypeptide wherein X₄ in sequence i) is selected from D and Q. For avoidance of doubt, the listed embodiments may be freely combined in yet other embodiments. For example, one such combined embodiment is a polypeptide in which X₄ is selected from A, D and Q, while X₇ is selected from E, H and W, and X₁₈ is selected from A, E, Q, R and T.

TABLE 1

| Xₙ | Possible residues |
|---|---|
| X₂ | A, D, S and T |
| X₂ | A, D and S |
| X₂ | A, D and T |
| X₂ | A and D |
| X₂ | A |
| X₂ | D |
| X₃ | E, R and W |
| X₃ | E and W |
| X₃ | R andW |
| X₃ | W |
| X₃ | R |
| X₃ | E |
| X₄ | A, D and Q |
| X₄ | D and Q |
| X₄ | A |
| X₄ | D |
| X₄ | Q |
| X₆ | F and M |
| X₆ | F |
| X₆ | M |
| X₇ | E, H, W and Y |
| X₇ | E, H and W |
| X₇ | E, H and Y |
| X₇ | E, W and Y |
| X₇ | H, W and Y |
| X₇ | W and Y |
| X₇ | H and W |
| X₇ | H and Y |
| X₇ | E and W |
| X₇ | E and Y |
| X₇ | E |
| X₇ | H |
| X₇ | W |
| X₇ | Y |
| X₁₀ | I and L |
| X₁₀ | I |
| X₁₀ | L |
| X₁₁ | A and W |
| X₁₁ | A |
| X₁₁ | W |
| X₁₆ | N and T |
| X₁₆ | N |
| X₁₆ | T |
| X₁₇ | A, D, N and W |
| X₁₇ | D, N and W |
| X₁₇ | D and N |
| X₁₇ | D and W |
| X₁₇ | A |
| X₁₇ | D |
| X₁₇ | N |
| X₁₇ | W |
| X₁₈ | A, E, Q, R, S, T and Y |
| X₁₈ | A, E, Q, R, T and Y |
| X₁₈ | A, E, Q, R and Y |
| X₁₈ | A, E, Q, R and T |
| X₁₈ | A, E and Q |
| X₁₈ | A, E and T |
| X₁₈ | A, Q and R |
| X₁₈ | A and E |
| X₁₈ | A |
| X₁₈ | E |
| X₁₈ | Q |
| X₁₈ | R |
| X₁₈ | S |
| X₁₈ | T |
| X₁₈ | Y |
| X₂₀ | A and Q |
| X₂₀ | A |
| X₂₀ | Q |
| X₂₁ | K, L and R |
| X₂₁ | K and R |
| X₂₁ | K |
| X₂₁ | L |
| X₂₁ | R |
| X₂₆ | K and S |
| X₂₆ | K |
| X₂₆ | S |

As described in detail in the experimental section to follow, the selection of HMGB1 binding polypeptide variants has led to the identification of a number of individual HMGB1 binding motif (BM) sequences belonging to the class defined in the first aspect of the disclosure. These sequences constitute individual embodiments of sequence i) according to this aspect. The sequences of individual HMGB1 binding motifs correspond to amino acid positions 8-36 in SEQ ID NO:1-32 presented in FIG. 1. In one embodiment of the HMGB1 binding polypeptide according to this first aspect, sequence i) corresponds to the sequence from position 8 to position 36 in a sequence selected from the group consisting of SEQ ID NO:1-14, 16 and 20-23. In one embodiment, sequence i) corresponds to the sequence from position 8 to position 36 in a sequence selected from the group consisting of SEQ ID NO:1-14. In one embodiment, sequence i) corresponds to the sequence from position 8 to position 36 in a sequence selected from the group consisting of SEQ ID NO:1-10. In one embodiment, sequence i) corresponds to the sequence from position 8 to position 36 in a sequence selected from the group consisting of SEQ ID NO:1-3. In one embodiment, sequence i) corresponds to the sequence from position 8 to position 36 in a sequence selected from the group consisting of SEQ ID NO:4-13. In one embodiment, sequence i) corresponds to the sequence from position 8 to position 36 in SEQ ID NO:1.

In some embodiments, the HMGB1 binding polypeptide of the first aspect binds to the A-box of HMGB1. In one such embodiment, the HMGB1 binding polypeptide of the first aspect binds to the A-box of HMGB1 in its reduced state. In another embodiment, the HMGB1 binding polypeptide of the first aspect binds to the A-box of HMGB1 in its oxidized state. In yet another embodiment, the HMGB1 binding polypeptide of the first aspect binds to the A-box of HMGB1 in both its reduced and oxidized states.

In one embodiment, the HMGB1 binding polypeptide of the first aspect binds to the A-box of HMGB1 such that the K_{D} value of the interaction is at most 1×10⁻⁶ M, for example at most 5×10⁻⁷ M, for example at most 1×10⁻⁷ M, for example at most 5×10⁻⁸ M, for example at most 1×10⁻⁸ M.

In one embodiment, the HMGB1 binding polypeptide of the first aspect binds to the A-box of HMGB1 such that the half maximal effective concentration of the interaction (EC50), in a suitable assay of the effect, is at most 1×10⁻⁶ M, for example at most 5×10⁻⁷ M, for example at most 1×10⁻⁷ M, for example at most 5×10⁻⁸ M, for example at most 1×10⁻⁸ M.

In a second aspect of the disclosure, there is provided an HMGB1 binding polypeptide, comprising an HMGB1 binding motif BM, which motif consists of an amino acid sequence selected from:

iii)

(SEQ ID NO: 57)
EAWX₄AEQEIW X₁₁LPNLX₁₆X₁₇X₁₈QF QAFX₂₅X₂₆LX₂₈D

wherein each of X₄, X₁₁, X₁₇, X₁₈, X₂₅, and X₂₈ are, independently of each other, selected from A, D, E, F, G, H, I, K, L, M, N, Q, R, S, T, V,
W and Y;
X₁₆ is selected from N and T; and
X₂₆ is selected from K and S;
   and
   iv) an amino acid sequence which has at least 93% identity to the sequence defined in iii).

In another embodiment, there is provided an HMGB1 binding polypeptide wherein in sequence iii)
X₄ is selected from L and S;
X₁₁ is selected from D and Q;
X₁₆ is selected from N and T;
X₁₇ is selected from E and L;
X₁₈ is selected from A and Q;
X₂₅ is selected from L and M;
X₂₆ is selected from K and S; and
X₂₈ is selected from I and L.

As used herein, “Xₙ” and “Xₘ” are used to indicate amino acids in positions n and m in the sequence iii) as defined above, wherein n and m are integers which indicate the position of an amino acid within said sequence as counted from the N-terminal end of said sequence. For example, X₄ and X₁₁ indicate the amino acid in position four and eleven, respectively, from the N-terminal end of sequence iii).

In embodiments according to the second aspect, there are provided polypeptides wherein Xₙ in sequence iii) is independently selected from a group of possible residues according to Table 2. The skilled person will appreciate that Xₙ may be selected from any one of the listed groups of possible residues and that this selection is independent from the selection of amino acids in Xₘ, wherein n≠m. Thus, any of the listed possible residues in position Xₙ in Table 2 may be independently combined with any of the listed possible residues any other variable position in Table 2, in analogy to what is explained above in connection with the first aspect of the disclosure and Table 1.

TABLE 2

| Xₙ | Possible residues |
|---|---|
| X₄ | A, D, E, F, G, H, I, K, L, M, |
| | N, Q, R, S, T, V, W and Y |
| X₄ | A, I, L, S, T and V |
| X₄ | A, I, L, S and V |
| X₄ | A, I, L and S |
| X₄ | I, L, S, T and V |
| X₄ | I, L, S and V |
| X₄ | I, L and V |
| X₄ | I, L and S |
| X₄ | I and L |
| X₄ | L and V |
| X₄ | A, S and T |
| X₄ | S and T |
| X₄ | L and S |
| X₄ | L |
| X₄ | S |
| X₁₁ | A, D, E, F, G, H, I, K, L, M, |
| | N, Q, R, S, T, V, W and Y |
| X₁₁ | A, D, E, N and Q |
| X₁₁ | A, D, E and Q |
| X₁₁ | D, E and Q |
| X₁₁ | D and E |
| X₁₁ | E and Q |
| X₁₁ | D and Q |
| X₁₁ | D |
| X₁₁ | Q |
| X₁₆ | N and T |
| X₁₆ | N |
| X₁₆ | T |
| X₁₇ | A, D, E, F, G, H, I, K, L, M, |
| | N, Q, R, S, T, V, W and Y |
| X₁₇ | D, E, I, L, Q and V |
| X₁₇ | D, E, I, L and V |
| X₁₇ | D, E, I and L |
| X₁₇ | E, I and L |
| X₁₇ | D, E and L |
| X₁₇ | I, L and V |
| X₁₇ | D and E |
| X₁₇ | I and L |
| X₁₇ | E and L |
| X₁₇ | E |
| X₁₇ | L |
| X₁₈ | A, D, E, F, G, H, I, K, L, M, |
| | N, Q, R, S, T, V, W and Y |
| X₁₈ | A, D, E, I, L, N, Q and V |
| X₁₈ | A, D, E, L, N, Q and V |
| X₁₈ | A, D, E, N, Q and V |
| X₁₈ | A, D, E, N and Q |
| X₁₈ | A, D, N and Q |
| X₁₈ | A, N and Q |
| X₁₈ | A, D and Q |
| X₁₈ | A and Q |
| X₁₈ | A |
| X₁₈ | Q |
| X₂₅ | A, D, E, F, G, H, I, K, L, M, |
| | N, Q, R, S, T, V, W and Y |
| X₂₅ | I, L, M and V |
| X₂₅ | I, L and M |
| X₂₅ | I and M |
| X₂₅ | I and L |
| X₂₅ | L and M |
| X₂₅ | L |
| X₂₅ | M |
| X₂₆ | K and S |
| X₂₆ | K |
| X₂₆ | S |
| X₂₈ | A, D, E, F, G, H, I, K, L, M, |
| | N, Q, R, S, T, V, W and Y |
| X₂₈ | A, I, L, V, W and Y |
| X₂₈ | A, I, L, V and W |
| X₂₈ | A, I, L, V and Y |
| X₂₈ | A, I, L and V |
| X₂₈ | I, L and V |
| X₂₈ | I and V |
| X₂₈ | L and V |
| X₂₈ | I and L |
| X₂₈ | I |
| X₂₈ | L |
| X₂₈ | V |

As described in detail in the experimental section to follow, the selection of HMGB1 binding polypeptide variants has led to the identification of a number of individual HMGB1 binding motif (BM) sequences belonging to the class defined in the second aspect of the disclosure. These sequences constitute individual embodiments of sequence iii) according to this aspect. The sequences of individual HMGB1 binding motifs correspond to amino acid positions 8-36 in SEQ ID NO:1-32 presented in FIG. 1. In one embodiment of the HMGB1 binding polypeptide according to this second aspect, sequence iii) corresponds to the sequence from position 8 to position 36 in a sequence selected from the group consisting of SEQ ID NO:30 and 31. In one embodiment, sequence iii) corresponds to the sequence from position 8 to position 36 in SEQ ID NO:30. In one embodiment, sequence iii) corresponds to the sequence from position 8 to position 36 in SEQ ID NO:31.

In some embodiments, the HMGB1 binding polypeptide of the second aspect binds to the B-box of HMGB1. In one such embodiment, the HMGB1 binding polypeptide of the second aspect binds to the B-box of HMGB1 in its reduced state. In another embodiment, the HMGB1 binding polypeptide of the second aspect binds to the B-box of HMGB1 in its oxidized state. In yet another embodiment, the HMGB1 binding polypeptide of the second aspect binds to the B-box of HMGB1 in both its reduced and oxidized states.

In one embodiment, the HMGB1 binding polypeptide of the second aspect binds to the B-box of HMGB1 such that the K_{D} value of the interaction is at most 1×10⁻⁶ M, for example at most 5×10⁻⁷ M, for example at most 1×10⁻⁷ M, for example at most 5×10⁻⁸ M, for example at most 1×10⁻⁸ M.

In one embodiment, the HMGB1 binding polypeptide of the second aspect binds to the B-box of HMGB1 such that the half maximal effective concentration of the interaction (EC50), in a suitable assay of the effect, is at most 1×10⁻⁶ M, for example at most 5×10⁻⁷ M, for example at most 1×10⁻⁷ M, for example at most 5×10⁻⁸ M, for example at most 1×10⁻⁸ M.

The above definitions of two separate classes of sequence related, HMGB1 binding polypeptides are based on a statistical analysis of a number of random polypeptide variants of a parent scaffold, that were selected for their interaction with of HMGB1 in selection experiments (A-box and/or B-box and/or entire HMGB1 protein). The identified HMGB1 binding motifs, or “BM”, correspond to the target binding region of the parent scaffold, which region constitutes two alpha helices within a three-helical bundle protein domain. In the parent scaffold, the varied amino acid residues of the two BM helices constitute a binding surface for interaction with the constant Fc part of antibodies. In the present disclosure, the random variation of binding surface residues and subsequent selection of variants have replaced the Fc interaction capacity with a capacity for interaction with HMGB1.

As the skilled person will realize, the function of any polypeptide, such as the HMGB1 binding capacity of the polypeptide of the present disclosure, is dependent on the tertiary structure of the polypeptide. It is therefore possible to make minor changes to the sequence of amino acids in a polypeptide without affecting the function thereof. Thus, the disclosure encompasses modified variants of the HMGB1 binding polypeptide, which have retained HMGB1 binding characteristics.

In this way, encompassed by the present disclosure is an HMGB1 binding polypeptide comprising an amino acid sequence with 93% or greater identity, such as 96% or greater identity, to a polypeptide as defined in i) or to a polypeptide defined in iii). For example, it is possible that an amino acid residue belonging to a certain functional grouping of amino acid residues (e.g. hydrophobic, hydrophilic, polar etc.) could be exchanged for another amino acid residue from the same functional group.

In some embodiments, such changes may be made in any position of the sequence of the HMGB1 binding polypeptide as disclosed herein. In other embodiments, such changes may be made only in the non-variable positions, also denoted scaffold amino acid residues. In such cases, changes are not allowed in the variable positions. In other embodiments, such changes may be only in the variable positions. According to one definition of such “variable positions”, these are positions denoted with an “X” in sequence i) or iii) as defined above. According to another definition, “variable positions” are those positions that are randomized in a selection library of Z variants prior to selection, and may thus for example be positions 2, 3, 4, 6, 7, 10, 11, 17, 18, 20, 21, 25 and 28 in sequence i) or sequence iii), as illustrated in Example 1.

The term “% identity”, as used throughout the specification, may for example be calculated as follows. The query sequence is aligned to the target sequence using the CLUSTAL W algorithm (Thompson et al (1994) Nucleic Acids Research, 22: 4673-4680). A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % identity.

In some embodiments of either the first or second aspect of the present disclosure, the BM as defined above “forms part of” a three-helix bundle protein domain. This is understood to mean that the sequence of the BM is “inserted” into or “grafted” onto the sequence of the original three-helix bundle domain, such that the BM replaces a similar structural motif in the original domain. For example, without wishing to be bound by theory, the BM is thought to constitute two of the three helices of a three-helix bundle, and can therefore replace such a two-helix motif within any three-helix bundle. As the skilled person will realize, the replacement of two helices of the three-helix bundle domain by the two BM helices has to be performed so as not to affect the basic structure of the polypeptide. That is, the overall folding of the Ca backbone of the polypeptide according to this embodiment of the invention is substantially the same as that of the three-helix bundle protein domain of which it forms a part, e.g. having the same elements of secondary structure in the same order etc. Thus, a BM according to the present disclosure “forms part” of a three-helix bundle domain if the polypeptide according to this embodiment has the same fold as the original domain, implying that the basic structural properties are shared, those properties e.g. resulting in similar CD spectra. The skilled person is aware of other parameters that are relevant.

In particular embodiments, the HMGB1 binding motif (BM) thus forms part of a three-helix bundle protein domain. For example, the BM may essentially constitute two alpha helices with an interconnecting loop, within said three-helix bundle protein domain. In particular embodiments, said three-helix bundle protein domain is selected from domains of bacterial receptor proteins. Non-limiting examples of such domains are the five different three-helical domains of Protein A from Staphylococcus aureus, such as domain B, and derivatives thereof. In some embodiments, the three-helical bundle protein domain is a variant of protein Z, which is derived from domain B of staphylococcal Protein A (Wahlberg E et al 2003, PNAS 100(6):3185-3190).

In some embodiments where the HMGB1 binding polypeptide as disclosed herein forms part of a three-helix bundle protein domain, the HMGB1 binding polypeptide may comprise a binding module (BMod), the amino acid sequence of which is selected from:

v)

(SEQ ID NO: 58)
K-[BM]-DPSQSXₐX_{b}LLX_{c} EAKKLX_{d}XₑX_{f}Q;

wherein
[BM] is an HMGB1 binding motif according to any definition herein;
Xₐ is selected from A and S;
X_{b} is selected from N and E;
X_{c} is selected from A, S and C;
X_{d} is selected from E, N and S;
Xₑ is selected from D, E and S; and
X_{f} is selected from A and S; and
   vi) an amino acid sequence which has at least 93% identity to a sequence defined in v).

In some embodiments, said polypeptide may beneficially exhibit a high structural stability, such as resistance to chemical modifications, to changes in physical conditions and to proteolysis, during production and storage, as well as in vivo.

As discussed above, polypeptides comprising minor changes as compared to the above amino acid sequences, which do not largely affect the tertiary structure and the function of the polypeptide, are also within the scope of the present disclosure. Thus, in some embodiments, sequence vi) has at least 93%, such as at least 95%, such as at least 97% identity to a sequence defined by v).

In one embodiment, Xₐ in sequence v) is A.
In one embodiment, Xₐ in sequence v) is S.
In one embodiment, X_{b} in sequence v) is N.
In one embodiment, X_{b} in sequence v) is E.
In one embodiment, X_{c} in sequence v) is A.
In one embodiment, X_{c} in sequence v) is S.
In one embodiment, X_{c} in sequence v) is C.
In one embodiment, X_{d} in sequence v) is E.
In one embodiment, X_{d} in sequence v) is N.
In one embodiment, X_{d} in sequence v) is S.
In one embodiment, Xₑ in sequence v) is D.
In one embodiment, Xₑ in sequence v) is E.
In one embodiment, Xₑ in sequence v) is S.
In one embodiment, X_{d}Xₑ in sequence v) is selected from EE, ES, SD, SE and SS.
In one embodiment, X_{d}Xₑ in sequence v) is ES.
In one embodiment, X_{d}Xₑ in sequence v) is SE.
In one embodiment, X_{d}Xₑ in sequence v) is SD.
In one embodiment, X_{f} in sequence v) is A.
In one embodiment, X_{f} in sequence v) is S.
In one embodiment, in sequence v), Xₐ is A; X_{b} is N; X_{c} is A and X_{f} is A.
In one embodiment, in sequence v), Xₐ is S; X_{b} is E; X_{c} is A and X_{f} is A.
In one embodiment, in sequence v), Xₐ is A; X_{b} is N; X_{c} is C and X_{f} is A.
In one embodiment, in sequence v), Xₐ is S; X_{b} is E; X_{c} is S and X_{f} is S.
In one embodiment, in sequence v), Xₐ is S; X_{b} is E; X_{c} is C and X_{f} is S.
In one embodiment, in sequence v), Xₐ is A; X_{b} is N; X_{c} is A; X_{d}Xₑ is ND and X_{f} is A.
In one embodiment, in sequence v), Xₐ is S; X_{b} is E; X_{c} is A; X_{d}Xₑ is ND and X_{f} is A.
In one embodiment, in sequence v), Xₐ is A; X_{b} is N; X_{c} is C; X_{d}Xₑ is ND and X_{f} is A.
In one embodiment, in sequence v), Xₐ is S; X_{b} is E; X_{c} is S; X_{d}Xₑ is ND and X_{f} is S.
In one embodiment, in sequence v), Xₐ is S; X_{b} is E; X_{c} is C; X_{d}Xₑ is ND and X_{f} is S.
In one embodiment, in sequence v), Xₐ is A; X_{b} is N; X_{c} is A; X_{d}Xₑ is SE and X_{f} is A.
In one embodiment, in sequence v), Xₐ is S; X_{b} is E; X_{c} is A; X_{d}Xₑ is SE and X_{f} is A.
In one embodiment, in sequence v), Xₐ is A; X_{b} is N; X_{c} is C; X_{d}Xₑ is SE and X_{f} is A.
In one embodiment, in sequence v), Xₐ is S; X_{b} is E; X_{c} is S; X_{d}Xₑ is SE and X_{f} is S.
In one embodiment, in sequence v), Xₐ is S; X_{b} is E; X_{c} is C; X_{d}Xₑ is SE and X_{f} is S.
In one embodiment, in sequence v), Xₐ is A; X_{b} is N; X_{c} is A; X_{d}Xₑ is SD and X_{f} is A.
In one embodiment, in sequence v), Xₐ is S; X_{b} is E; X_{c} is A; X_{d}Xₑ is SD and X_{f} is A.
In one embodiment, in sequence v), Xₐ is A; X_{b} is N; X_{c} is C; X_{d}Xₑ is SD and X_{f} is A.
In one embodiment, in sequence v), Xₐ is S; X_{b} is E; X_{c} is S; X_{d}Xₑ is SD and X_{f} is S.
In one embodiment, in sequence v), Xₐ is S; X_{b} is E; X_{c} is C; X_{d}Xₑ is SD and X_{f} is S.

In a further embodiment, which is an embodiment of the first aspect of the disclosure, sequence v) corresponds to the sequence from position 7 to position 55 in a sequence selected from the group consisting of SEQ ID NO:1-14, 16, 20-23 and 27-29. In one embodiment, sequence v) corresponds to the sequence from position 7 to position 55 in a sequence selected from the group consisting of SEQ ID NO:1-14 and 29. In one embodiment, sequence v) corresponds to the sequence from position 7 to position 55 in a sequence selected from the group consisting of SEQ ID NO:1-14. In one embodiment, sequence v) corresponds to the sequence from position 7 to position 55 in a sequence selected from the group consisting of SEQ ID NO:27-29. In one embodiment, sequence v) corresponds to the sequence from position 7 to position 55 in a sequence selected from the group consisting of SEQ ID NO:1-10. In one embodiment, sequence v) corresponds to the sequence from position 7 to position 55 in a sequence selected from the group consisting of SEQ ID NO:1-3. In one embodiment, sequence v) corresponds to the sequence from position 7 to position 55 in a sequence selected from the group consisting of SEQ ID NO:4-13. In one embodiment, sequence v) corresponds to the sequence from position 7 to position 55 in SEQ ID NO:1. In one embodiment, sequence v) corresponds to the sequence from position 7 to position 55 in SEQ ID NO:29.

In yet a further embodiment, which is an embodiment of the second aspect of the disclosure, sequence v) corresponds to the sequence from position 7 to position 55 in a sequence selected from the group consisting of SEQ ID NO:30-32. In one embodiment, sequence v) corresponds to the sequence from position 7 to position 55 in SEQ ID NO:31.

Also, in a further embodiment, there is provided an HMGB1 binding polypeptide, which comprises an amino acid sequence selected from:

vii) YA-[BMod]-AP;

wherein [BMod] is an HMGB1 binding module as defined herein; and

viii) an amino acid sequence which has at least 90% identity to a sequence defined in v).

In one embodiment, the HMGB1 binding polypeptide comprises an amino acid sequence selected from:

ix)

(SEQ ID NO: 59)
VDAKYAK-[BM]-DPSQSSELLSEAKKLNDSQAPK;

wherein [BM] is an HMGB1 binding motif as defined herein; and

x) an amino acid sequence which has at least 89% identity to the sequence defined in ix).

In a further embodiment, which is an embodiment of the first aspect of the disclosure, sequence ix) is selected from SEQ ID NO:1-3 and 14-26. In another such embodiment, sequence ix) is selected from SEQ ID NO:1-3. In another such embodiment, sequence ix) is SEQ ID NO:1.

In a further embodiment, which is an embodiment of the second aspect of the disclosure, sequence ix) is selected from SEQ ID NO:30-31. In another such embodiment, sequence ix) is SEQ ID NO:31.

In another further embodiment, the HMGB1 binding polypeptide comprises an amino acid sequence selected from:

xi)

(SEQ ID NO: 60)
AEAKYAK-[BM]-DPSQSSELLSEAKKLNDSQAPK,

wherein [BM] is an HMGB1 binding motif as defined herein; and
xii) an amino acid sequence which has at least 89% identity to the sequence defined in xi).

In a further embodiment, which is an embodiment of the first aspect of the disclosure, sequence xi) is selected from SEQ ID NO:27-28. In another such embodiment, sequence xi) is SEQ ID NO:27.

In a further embodiment, which is an embodiment of the second aspect of the disclosure, sequence xi) is SEQ ID NO:32.

Also, in a further embodiment, there is provided an HMGB1 binding polypeptide, which comprises an amino acid sequence selected from:

xiii) FA-[BMod]-AP;

wherein [BMod] is an HMGB1 binding module as defined herein; and

xiv) an amino acid sequence which has at least 90% identity to a sequence defined in xiii).

In one embodiment, the HMGB1 binding polypeptide comprises an amino acid sequence selected from:

xv)

(SEQ ID NO: 61)
AEAKFAK-[BM]-DPSQSSELLSEAKKLSESQAPK,

wherein [BM] is an HMGB1 binding motif as defined herein; and

xvi) an amino acid sequence which has at least 89% identity to the sequence defined in xv).

In a further embodiment, which is an embodiment of the first aspect of the disclosure, sequence xv) is selected from SEQ ID NO:4-13 and 29. In another such embodiment, sequence ix) is SEQ ID NO:29.

In one embodiment, the HMGB1 binding polypeptide comprises an amino acid sequence selected from:

xvii)

(SEQ ID NO: 62)
AEAKFAK-[BM]-DPSQSSELLSEAKKLNDSQAPK;

wherein [BM] is an HMGB1 binding motif as defined herein; and

xviii) an amino acid sequence which has at least 89% identity to the sequence defined in xxiii).

As discussed above, polypeptides comprising minor changes as compared to the above amino acid sequences, which do not largely affect the tertiary structure and the function of the polypeptide, also fall within the scope of the present disclosure. Thus, in some embodiments, sequence vi), viii), x), xii), xiv), xvi) or xviii) may for example be at least 89%, such as at least 90%, such as at least 91%, such as at least 92%, such as at least 93%, such as at least 94%, such as at least 95%, such as at least 96%, such as at least 97%, such as at least 98%, such as at least 99% identical to a sequence defined by v), vii), ix), xi), xiii), xv) and xvii), respectively.

In some embodiments, the HMGB1 binding motif may form part of a polypeptide comprising an amino acid sequence selected from

(SEQ ID NO: 63)
ADNNFNK-[BM]-DPSQSANLLSEAKKLNESQAPK;
(SEQ ID NO: 64)
ADNKFNK-[BM]-DPSQSANLLAEAKKLNDAQAPK;
(SEQ ID NO: 65)
ADNKFNK-[BM]-DPSVSKEILAEAKKLNDAQAPK;
(SEQ ID NO: 66)
ADAQQNNFNK-[BM]-DPSQSTNVLGEAKKLNESQAPK;
(SEQ ID NO: 67)
AQHDE-[BM]-DPSQSANVLGEAQKLNDSQAPK;
(SEQ ID NO: 68)
VDNKFNK-[BM]-DPSQSANLLAEAKKLNDAQAPK;
(SEQ ID NO: 69)
AEAKYAK-[BM]-DPSESSELLSEAKKLNKSQAPK;
(SEQ ID NO: 70)
VDAKYAK-[BM]-DPSQSSELLAEAKKLNDAQAPK;
(SEQ ID NO: 71)
VDAKYAK-[BM]-DPSQSSELLAEAKKLNDSQAPK;
(SEQ ID NO: 72)
AEAKYAK-[BM]-DPSQSSELLSEAKKLNDSQAPK;
(SEQ ID NO: 73)
AEAKFAK-[BM]-DPSQSSELLSEAKKLNDSQAPK;
(SEQ ID NO: 74)
AEAKFAK-[BM]-DPSQSSELLSEAKKLNDSQAPK;
(SEQ ID NO: 75)
AEAKFAK-[BM]-DPSQSSELLSEAKKLNDSQAP;
(SEQ ID NO: 76)
AEAKYAK-[BM]-DPSQSSELLAEAKKLNDAQAPK;
(SEQ ID NO: 77)
AEAKYAK-[BM]-DPSQSSELLSEAKKLSESQAPK;
(SEQ ID NO: 78)
AEAKYAK-[BM]-DPSQSSELLSEAKKLSESQAP;
(SEQ ID NO: 79)
AEAKFAK-[BM]-DPSQSSELLSEAKKLSESQAPK;
(SEQ ID NO: 80)
AEAKFAK-[BM]-DPSQSSELLSEAKKLSESQAP;
(SEQ ID NO: 81)
AEAKYAK-[BM]-DPSQSSELLAEAKKLSEAQAPK;
(SEQ ID NO: 82)
AEAKYAK-[BM]-QPEQSSELLSEAKKLSESQAPK;
(SEQ ID NO: 83)
AEAKYAK-[BM]-DPSQSSELLSEAKKLESSQAPK;
(SEQ ID NO: 84)
AEAKYAK-[BM]-DPSQSSELLSEAKKLESSQAP;
(SEQ ID NO: 85)
AEAKYAK-[BM]-DPSQSSELLAEAKKLESAQAPK;
(SEQ ID NO: 86)
AEAKYAK-[BM]-QPEQSSELLSEAKKLESSQAPK;
(SEQ ID NO: 87)
AEAKYAK-[BM]-DPSQSSELLSEAKKLSDSQAPK;
(SEQ ID NO: 88)
AEAKYAK-[BM]-DPSQSSELLSEAKKLSDSQAP;
(SEQ ID NO: 89)
AEAKYAK-[BM]-DPSQSSELLAEAKKLSDSQAPK;
(SEQ ID NO: 90)
AEAKYAK-[BM]-DPSQSSELLAEAKKLSDAQAPK;
(SEQ ID NO: 91)
AEAKYAK-[BM]-QPEQSSELLSEAKKLSDSQAPK;
(SEQ ID NO: 92)
VDAKYAK-[BM]-DPSQSSELLSEAKKLNDSQAPK;
(SEQ ID NO: 93)
VDAKYAK-[BM]-DPSQSSELLAEAKKLNDAQAPK;
(SEQ ID NO: 94)
VDAKYAK-[BM]-DPSQSSELLSEAKKLSESQAPK;
(SEQ ID NO: 95)
VDAKYAK-[BM]-DPSQSSELLAEAKKLSEAQAPK;
(SEQ ID NO: 96)
VDAKYAK-[BM]-QPEQSSELLSEAKKLSESQAPK;
(SEQ ID NO: 97)
VDAKYAK-[BM]-DPSQSSELLSEAKKLESSQAPK;
(SEQ ID NO: 98)
VDAKYAK-[BM]-DPSQSSELLAEAKKLESAQAPK;
(SEQ ID NO: 99)
VDAKYAK-[BM]-QPEQSSELLSEAKKLESSQAPK;
(SEQ ID NO: 100)
VDAKYAK-[BM]-DPSQSSELLSEAKKLSDSQAPK;
(SEQ ID NO: 101)
VDAKYAK-[BM]-DPSQSSELLAEAKKLSDSQAPK;
(SEQ ID NO: 102)
VDAKYAK-[BM]-DPSQSSELLAEAKKLSDAQAPK;
(SEQ ID NO: 103)
VDAKYAK-[BM]-QPEQSSELLSEAKKLSDSQAPK;
(SEQ ID NO: 104)
VDAKYAK-[BM]-DPSQSSELLAEAKKLNKAQAPK;
(SEQ ID NO: 105)
AEAKYAK-[BM]-DPSQSSELLAEAKKLNKAQAPK;
and
(SEQ ID NO: 106)
ADAKYAK-[BM]-DPSQSSELLSEAKKLNDSQAPK;

wherein [BM] is an HMGB1 binding motif as defined herein.

In one embodiment, said HMGB1 is human HMGB1. In another embodiment, said HMGB1 is rat HMGB1.

The terms “HMGB1 binding” and “binding affinity for HMGB1” as used in this specification refer to a property of a polypeptide which may be tested for example by ELISA, by the use of surface plasmon resonance (SPR) technology, or by the use of quartz crystal microbalance (QCM) technology.

For example as described in the experimental section below, HMGB1 binding affinity may be tested in an experiment in which samples of the polypeptide are captured on antibody-coated ELISA plates and biotinylated HMGB1 is added followed by streptavidin-conjugated HRP. TMB substrate is added and the absorbance at 450 nm is measured using a multi-well plate reader, such as Victor³ (Perkin Elmer). The skilled person may then interpret the results obtained by such experiments to establish at least a qualitative measure of the binding affinity of the polypeptide for HMGB1. If a quantitative measure is desired, for example to determine the EC50 value (the half maximal effective concentration) for the interaction, ELISA may also be used. The response of the polypeptide against a dilution series of biotinylated HMGB1 is measured using ELISA as described above. The skilled person may then interpret the results obtained by such experiments, and EC50 values may be calculated from the results using for example GraphPad Prism 5 and non-linear regression.

HMGB1 binding affinity may also be tested in an experiment in which HMGB1, or a fragment thereof, is immobilized on a sensor chip of a surface plasmon resonance (SPR) instrument, and the sample containing the polypeptide to be tested is passed over the chip. Alternatively, the polypeptide to be tested is immobilized on a sensor chip of the instrument, and a sample containing HMGB1, or a fragment thereof, is passed over the chip. The skilled person may then interpret the results obtained by such experiments to establish at least a qualitative measure of the binding affinity of the polypeptide for HMGB1. If a quantitative measure is desired, for example to determine a K_{D} value for the interaction, surface plasmon resonance methods may also be used. Binding values may for example be defined in a Biacore (GE Healthcare) or ProteOn XPR 36 (Bio-Rad) instrument. HMGB1 is suitably immobilized on a sensor chip of the instrument, and samples of the polypeptide whose affinity is to be determined are prepared by serial dilution and injected in random order. K_{D} values may then be calculated from the results using for example the 1:1 Langmuir binding model of the BIAevaluation 4.1 software, or other suitable software, provided by the instrument manufacturer.

HMGB1 binding affinity may also be tested using a continuous-flow system for automated analysis based on the Quartz Crystal Microbalance (QCM) technology. To monitor binding interactions, one of the interacting molecules, or fragment thereof, is immobilized on the sensor surface and the sample containing the other one is injected over the sensor surface. Binding data is measured in real-time. The signal output is given in frequency (Hz) and is directly related to changes in mass on the sensor surface. If a quantitative measure is desired, for example to determine a K_{D} value for the interaction, QCM methods may also be used. Binding values may for example be defined in an Attana A200® (Attana) instrument. For example, HMGB1 may be immobilized on a sensor chip of the instrument, and samples of the polypeptide whose affinity is to be determined are prepared by serial dilution and injected. Data is collected by Attester Software and K_{D} values may then be calculated from the results using for example a 1:1 Langmuir binding model and subsequently processed in the Evaluation Software, or other suitable software, provided by the instrument manufacturer.

The terms “albumin binding” and “binding affinity for albumin” as used in this disclosure refer to a property of a polypeptide which may be tested for example by the use of SPR technology in a Biacore instrument or ProteOn XPR36 instrument or by the use of QCM technology in an Attana instrument, in an analogous way to the examples described above for HMGB1.

The skilled person will understand that various modifications and/or additions can be made to an HMGB1 binding polypeptide according to any aspect disclosed herein in order to tailor the polypeptide to a specific application without departing from the scope of the present disclosure.

For example, in one embodiment, there is provided an HMGB1 binding polypeptide as described herein, which polypeptide has been extended by and/or comprises additional amino acids at the C terminus and/or N terminus. Such a polypeptide should be understood as a polypeptide having one or more additional amino acid residues at the very first and/or the very last position in the polypeptide chain. Thus, an HMGB1 binding polypeptide may comprise any suitable number of additional amino acid residues, for example at least one additional amino acid residue. Each additional amino acid residue may individually or collectively be added in order to, for example, improve and/or simplify production, purification, stabilization in vivo or in vitro, coupling or detection of the polypeptide. Such additional amino acid residues may comprise one or more amino acid residues added for the purpose of chemical coupling. One example of this is the addition of a cysteine residue. Additional amino acid residues may also provide a “tag” for purification or detection of the polypeptide, such as a His₆ tag, a (HisGlu)₃ tag (“HEHEHE” tag) or a “myc” (c-myc) tag or a “FLAG” tag for interaction with antibodies specific to the tag or immobilized metal affinity chromatography (IMAC) in the case of a His₆-tag.

In one embodiment, there is provided an HMGB1 binding polypeptide as described herein which comprises additional amino acids at the C-terminal and/or N-terminal end. For example, in one embodiment of the HMGB1 binding polypeptide as disclosed herein, it consists of any one of the sequences disclosed herein, having from 0 to 15 additional C-terminal and/or N-terminal residues, such as from 0 to 7 additional C-terminal and/or N-terminal residues. In one embodiment, the HMGB1 binding polypeptide consists of any one of the sequences disclosed herein, having from 0 to 15, such as from 0 to 4, such as 3 additional C-terminal residues. In one particular embodiment, the HMGB1 binding polypeptide as described herein comprises the additional C-terminal residues VDC, VEC or GC.

The further amino acids as discussed above may be coupled to the HMGB1 binding polypeptide by means of chemical conjugation (using known organic chemistry methods) or by any other means, such as expression of the HMGB1 binding polypeptide as a fusion protein or joined in any other fashion, either directly or via a linker, for example an amino acid linker.

A further polypeptide domain may moreover provide another HMGB1 binding moiety. Thus, in a further embodiment, there is provided an HMGB1 binding polypeptide in a multimeric form. Said multimer is understood to comprise at least two HMGB1 binding polypeptides as disclosed herein as monomer units, the amino acid sequences of which may be the same or different. Multimeric forms of the polypeptides may comprise a suitable number of domains, each having an HMGB1 binding motif, and each forming a monomer within the multimer. These domains may have the same amino acid sequence, but alternatively, they may have different amino acid sequences. In other words, the HMGB1 binding polypeptide of the invention may form homo- or heteromultimers, for example homo- or heterodimers. In one embodiment, there is provided an HMGB1 binding polypeptide, wherein said monomer units are covalently coupled together. In another embodiment, said HMGB1 binding polypeptide monomer units are expressed as a fusion protein. In one embodiment, there is provided an HMGB1 binding polypeptide in dimeric form. In one particular embodiment, said dimeric form is a homodimeric form. In another embodiment, said dimeric form is a heterodimeric form.

In a particular embodiment of an HMGB1 binding polypeptide in multimeric form, one of said at least two HMGB1 binding polypeptides as disclosed herein is present as a first monomeric unit and has affinity for the A-box of HMGB1, while another of said at least two HMGB1 binding polypeptides as disclosed herein is present as a second monomeric unit and has affinity for the B-box of HMGB1. In one embodiment, said first monomeric unit is an HMGB1 binding polypeptide according to the first aspect of the disclosure. In one embodiment, said second monomeric unit is an HMGB1 binding polypeptide according to the second aspect of the disclosure.

For the sake of clarity, throughout this disclosure, the term “HMGB1 binding polypeptide” is used to encompass HMGB1 binding polypeptides in all forms, i.e. monomeric and multimeric forms.

The further amino acids as discussed above may for example comprise one or more further polypeptide domain(s). A further polypeptide domain may provide the HMGB1 binding polypeptide with another function, such as for example another binding function, or an enzymatic function, or a toxic function or a fluorescent signaling function, or combinations thereof.

Furthermore, it may be beneficial that the HMGB1 binding polypeptide as defined herein is part of a fusion protein or a conjugate comprising a second or further moieties. Second and further moiety/moieties of the fusion polypeptide or conjugate in such a protein may suitably have a desired biological activity.

Thus, in a third aspect of the disclosure, there is provided a fusion protein or a conjugate, comprising a first moiety consisting of an HMGB1 binding polypeptide according to the first or second aspect, and a second moiety consisting of a polypeptide having a desired biological activity. In another embodiment, said fusion protein or conjugate may additionally comprise further moieties, comprising desired biological activities that can be either the same as or different from the biological activity of the second moiety.

Non-limiting examples of a desired biological activity comprise a therapeutic activity, a binding activity and an enzymatic activity. In one embodiment, the second moiety having a desired biological activity is a therapeutically active polypeptide. In one embodiment, said second moiety is an immune response modifying agent. In another embodiment, said second moiety is an anti-cancer agent.

In one embodiment of the first, second or third aspect of the disclosure, there is provided an HMGB1 binding polypeptide, fusion protein or conjugate which comprises an immune response modifying agent. Non-limiting examples of additional immune response modifying agents include immunomodulating agents or other anti-inflammatory agents

Non-limiting examples of therapeutically active polypeptides are biomolecules, such as molecules selected from the group consisting of human endogenous enzymes, hormones, growth factors, chemokines, cytokines and lymphokines.

Non-limiting examples of binding activities are binding activities which increase the in vivo half-life of the fusion protein or conjugate, and binding activities which act to block a biological activity. One example of such a binding activity is a binding activity, which increases the in vivo half-life of a fusion protein or conjugate. In one embodiment of said fusion protein or conjugate, the in vivo half-life of said fusion protein or conjugate is longer than the in vivo half-life of the HMGB1 binding polypeptide per se. In one embodiment, said in vivo half-life is increased at least 10 times, such as at least 25 times, such as at least 50 times, such as at least 75 times, such as at least 100 times, compared the in vivo half-life of the HMGB1 binding polypeptide per se.

As discussed, the fusion protein or conjugate may comprise at least one further moiety with a binding activity towards a target. In one particular embodiment, said target is albumin, binding to which increases the in vivo half-life of said fusion protein or conjugate. In one embodiment, said albumin binding activity is provided by an albumin binding domain (ABD) of streptococcal protein G, or a derivative thereof. Thus, said fusion protein may for example comprise an HMGB1 binding polypeptide in monomeric or multimeric form (such as a homodimeric or heterodimeric form) as defined herein and an albumin binding domain of streptococcal protein G or a derivative thereof. Derivatives of the albumin binding domain of streptococcal protein G are known to persons of skill in the art, for example from WO2009/016043, WO2012/004384 and WO2014/048977, all hereby incorporated by reference.

In another embodiment, there is provided a fusion protein or a conjugate wherein said second moiety having a desired binding activity is a protein based on protein Z, derived from the B domain of protein A from Staphylococcus aureus, which has a binding affinity for a target other than HMGB1.

In another embodiment, there is provided a fusion protein or a conjugate wherein said second moiety having a desired binding activity is an antibody or antigen binding fragment thereof. As is well known, antibodies are immunoglobulin molecules capable of specific binding to a target (an antigen), such as a carbohydrate, polynucleotide, lipid, polypeptide or other, through at least one antigen recognition site on the immunoglobulin molecule. As used herein, the term “antibody or an antigen binding fragment thereof” encompasses not only full-length or intact polyclonal or monoclonal antibodies, but also antigen-binding fragments thereof, such as Fab, Fab′, F(ab′)₂, Fab₃, Fv and variants thereof, fusion proteins comprising one or more antibody portions, humanized antibodies, chimeric antibodies, minibodies, diabodies, triabodies, tetrabodies, linear antibodies, single chain antibodies, multispecific antibodies (e.g. bispecific antibodies) and any other modified configuration of the immunoglobulin molecule that comprises an antigen recognition site of the required specificity, including glycosylation variants of antibodies, amino acid sequence variants of antibodies and covalently modified antibodies. Further examples of modified antibodies and antigen binding fragments thereof include nanobodies, AlbudAbs, DARTs (dual affinity re-targeting), BiTEs (bispecific T-cell engager), TandAbs (tandem diabodies), DAFs (dual acting Fab), two-in-one antibodies, SMIPs (small modular immunopharmaceuticals), FynomAbs (fynomers fused to antibodies), DVD-Igs (dual variable domain immunoglobulin), CovX-bodies (peptide modified antibodies), duobodies and triomAbs. This listing of variants of antibodies and antigen binding fragments thereof is not to be seen as limiting, and the skilled person is aware of other suitable variants.

In one embodiment, said at least one antibody or antigen binding fragment thereof is selected from the group consisting of full-length antibodies, Fab fragments, Fab′ fragments, F(ab′)₂ fragments, Fc fragments, Fv fragments, single chain Fv fragments, (scFv)₂ and domain antibodies. In one embodiment, said at least one antibody or antigen binding fragment thereof is selected from full-length antibodies, Fab fragments and scFv fragments. In one particular embodiment, said at least one antibody or antigen binding fragment thereof is a full-length antibody.

In one embodiment, the antibody or antigen binding fragment thereof is selected from the group consisting of monoclonal antibodies, human antibodies, humanized antibodies, chimeric antibodies, and antigen-binding fragments thereof.

For example, said fusion protein or conjugate, comprising at least one further moiety, may among many other possible configurations, comprise [HMGB1 binding polypeptide]-[albumin binding moiety]-[moiety with affinity for selected target], or [HMGB1 A-box binding polypeptide]-[HMGB1 B-box binding polypeptide]-[albumin binding moiety]. It is to be understood that the three moieties in this example may be arranged in any order from the N- to the C-terminal of the polypeptide.

The skilled person is aware that the construction of a fusion protein often involves the use of linkers between the functional moieties to be fused, and there are different kinds of linkers with different properties, such as flexible amino acid linkers, rigid amino acid linkers and cleavable amino acid linkers. Linkers have been used to for example increase stability or improve folding of fusion proteins, to increase expression, improve biological activity, enable targeting and alter pharmacokinetics of fusion proteins. Thus, in one embodiment, the polypeptide according to any aspect disclosed herein further comprises at least one linker, such as at least one linker selected from flexible amino acid linkers, rigid amino acid linkers and cleavable amino acid linkers. In one embodiment, said linker is arranged between said HMGB1 binding polypeptide and a further polypeptide domain, such as between an HMGB1 binding domain as disclosed herein and an antibody or antigen binding fragment thereof (as described in further detail below). Flexible linkers are often used in the art when the joined domains require a certain degree of movement or interaction, and may be particularly useful in some embodiments of the complex. Such linkers are generally composed of small, non-polar (for example G) or polar (for example S or T) amino acids. Some flexible linkers primarily consist of stretches of G and S residues, for example (GGGGS)ₚ. Adjusting the copy number “p” allows for optimization of linker in order to achieve appropriate separation between the functional moieties or to maintain necessary inter-moiety interaction. Apart from G and S linkers, other flexible linkers are known in the art, such as G and S linkers containing additional amino acid residues, such as T and A, to maintain flexibility, as well as polar amino acid residues to improve solubility. Additional non-limiting examples of linkers include ASGS (SEQ ID NO:130), GAPGGGGS (SEQ ID NO:107), GGGGSLVPRGSGGGGS (SEQ ID NO:108), (GS)₃ (SEQ ID NO:109), (GS)₄ (SEQ ID NO:110), (GS)₅ (SEQ ID NO:111), GGSGGHMGSGG (SEQ ID NO:112), GGSGGSGGSGG (SEQ ID NO:113), GGSGG (SEQ ID NO:114), GGSGGGGG (SEQ ID NO:115), GGGSEGGGSEGGGSEGGG (SEQ ID NO:116), AAGAATAA (SEQ ID NO:117), GGGGG (SEQ ID NO:118), GGSSG (SEQ ID NO:119), GSGGGTGGGSG (SEQ ID NO:120), GSGGGTGGGSG (SEQ ID NO:121), GSGSGSGSGGSG (SEQ ID NO:122), GSGGSGGSGGSGGS (SEQ ID NO:123) and GSGGSGSGGSGGSG (SEQ ID NO:124), respectively, and GT. The skilled person is aware of other suitable linkers.

In one embodiment, said linker is a flexible linker comprising glycine (G), serine (S) and/or threonine (T) residues. In one embodiment, said linker has a general formula selected from (GₙSₘ)ₚ and (SₙGₘ)ₚ, wherein, independently, n=1-7, m=0-7, n+m 8 and p=1-7. In one embodiment, n=1-5. In one embodiment, m=0-5. In one embodiment, p=1-5. In a more specific embodiment, n=4, m=1 and p=1-4. In one embodiment, said linker is selected from the group consisting of S₄G (SEQ ID NO:125), (S₄G)₃ (SEQ ID NO:126) and (S₄G)₄ (SEQ ID NO:127). In one embodiment, said linker is selected from the group consisting of G₄S (SEQ ID NO:128) and (G₄S)₃ (SEQ ID NO:129). In one particular embodiment, said linker is G₄S and in another embodiment said linker is (G₄S)₃.

With regard to the description above of fusion proteins or conjugates incorporating an HMGB1 binding polypeptide according to the disclosure, it is to be noted that the designation of first, second and further moieties is made for clarity reasons to distinguish between HMGB1 binding polypeptide or polypeptides according to the invention on the one hand, and moieties exhibiting the same or other functions on the other hand. These designations are not intended to refer to the actual order of the different domains in the polypeptide chain of the fusion protein or conjugate. Similarly, the designations first and second monomer units are made for clarity reasons to distinguish between said units. Thus, for example, said first moiety (or monomer unit) may without restriction appear at the N-terminal end, in the middle, or at the C-terminal end of the fusion protein or conjugate.

The above aspects furthermore encompass polypeptides in which the HMGB1 binding polypeptide according to the first or second aspect, or the HMGB1 binding polypeptide as comprised in a fusion protein or conjugate according to the third aspect, further comprises a label, such as a label selected from the group consisting of fluorescent dyes and metals, chromophoric dyes, chemiluminescent compounds, bioluminescent proteins, enzymes, radionuclides, radioactive particles and pretargeting recognition tags. Such labels may for example be used for detection of the polypeptide.

In embodiments in which the polypeptide, fusion protein or conjugate is labeled, directly or indirectly (e.g. via pretargeting), with an imaging agent (e.g. radioactive agent), measuring the amount or localization of labeled polypeptide may be done using imaging equipment, such as through acquiring radioactivity counts or images of radiation density, or derivatives thereof such as radiation concentration. Non-limiting examples of radionuclides, suitable for either direct labeling of the HMGB1 binding agent according to any aspect disclosed herein, or for indirect labeling by labeling of a complementary pretargeting moiety, include ⁶⁸Ga, ^{110m}In, ¹⁸F, ⁴⁵Ti, ⁴⁴Sc, ⁶¹Cu, ⁶⁶Ga, ⁶⁴Cu, ⁵⁵Co, ⁷²As, ⁸⁶Y, ⁸⁹Zr, ¹²⁴I, ⁷⁶Br, ¹¹¹In, ^{99m}Tc, ¹²³I, ¹³¹I and ⁶⁷Ga.

In one embodiment, the imaging equipment used in such measurements is positron emission tomography (PET) equipment, in which case the radionuclide is selected such that it is suitable for PET. The skilled person is aware of radionuclides suitable for use with PET. For example, a PET radionuclide is selected from the group consisting of ⁶⁸Ga, ^{110m}In, ¹⁸F, ⁴⁵Ti, ⁴⁴Sc, ⁶¹Cu, ⁶⁶Ga, ⁶⁴Cu, ⁵⁵Co, ⁷²As, ⁸⁶Y, ⁸⁹Zr, ¹²⁴I and ⁷⁶Br.

In another embodiment, the imaging equipment used is single-photon emission computed tomography (SPECT) equipment, in which case the radionuclide is selected such that it is suitable for SPECT. The skilled person is aware of radionuclides suitable for use with SPECT. For example, a SPECT radionuclide is selected from the group consisting of ¹¹¹In, ^{99m}Tc, ¹²³I, ¹³¹I and ⁶⁷Ga.

Thus, in one embodiment there is provided an HMGB1 binding polypeptide, fusion protein or complex as described herein, which comprises a direct or indirect radionuclide label, such as a radionuclide selected from the group consisting of ⁶⁸Ga, ^{110m}In, ¹⁸F, ⁴⁵Ti, ⁴⁴Sc, ⁶¹Cu, ⁶⁶Ga, ⁶⁴Cu, ⁵⁵CO, ⁷²As, ⁸⁶Y, ⁸⁹Zr, ¹²⁴I, ⁷⁶Br, ¹¹¹In, ^{99m}Tc, ¹²³I, ¹³¹I and ⁶⁷Ga, such as the group consisting of ⁶⁸Ga, ^{110m}In, ¹⁸F, ⁴⁵Ti, ⁴⁴Sc, ⁶¹Cu, ⁶⁶Ga, ⁶⁴Cu, ⁵⁵Co, ⁷²As, ⁸⁶Y, ⁸⁹Zr, ¹²⁴I and ⁷⁶Br, such as ¹⁸F.

In some embodiments, the labeled HMGB1 binding polypeptide is present as a moiety in a fusion protein or conjugate also comprising a second or further moiety having a desired biological activity. The label may in some instances be coupled only to the HMGB1 binding polypeptide, and in some instances both to the HMGB1 binding polypeptide and to the second moiety of the fusion protein or conjugate. Furthermore, it is also possible that the label may be coupled to a second moiety and not to the HMGB1 binding moiety. Hence, in yet another embodiment, there is provided an HMGB1 binding polypeptide comprising a second moiety, wherein said label is coupled to the second moiety only. Thus, when reference is made to a labeled polypeptide, this should be understood as a reference to all aspects of polypeptides as described herein, including HMGB1 binding polypeptides, fusion proteins and conjugates comprising an HMGB1 binding polypeptide.

In embodiments where the HMGB1 binding polypeptide, fusion protein or conjugate is radiolabeled, such a radiolabeled polypeptide may comprise a radionuclide. A majority of radionuclides have a metallic nature and metals are typically incapable of forming stable covalent bonds with elements presented in proteins and peptides. For this reason, labeling of proteins and peptides with radioactive metals is performed with the use of chelators, i.e. multidentate ligands, which form non-covalent compounds, called chelates, with the metal ions. In an embodiment of the HMGB1 binding polypeptide, fusion protein or conjugate, the incorporation of a radionuclide is enabled through the provision of a chelating environment, through which the radionuclide may be coordinated, chelated or complexed to the polypeptide. One example of a chelator is the polyaminopolycarboxylate type of chelator. Two classes of such polyaminopolycarboxylate chelators can be distinguished: macrocyclic and acyclic chelators.

In one embodiment, the HMGB1 binding polypeptide, fusion protein or conjugate comprises a chelating environment provided by a polyaminopolycarboxylate chelator conjugated to the HMGB1 binding polypeptide via a thiol group of a cysteine residue or an epsilon amine group of a lysine residue.

The most commonly used macrocyclic chelators for radioisotopes of indium, gallium, yttrium, bismuth, radioactinides and radiolanthanides are different derivatives of DOTA (1,4,7,10-tetraazacyclododecane-1,4,7,10-tetraacetic acid). In one embodiment, a chelating environment of the HMGB1 binding polypeptide, HMGB1 binding polypeptide in heterodimeric form, fusion protein or conjugate is provided by DOTA or a derivative thereof. More specifically, in one embodiment, a chelating polypeptide encompassed by the present disclosure is obtained by reacting the DOTA derivative 1,4,7,10-tetraazacyclododecane-1,4,7-tris-acetic acid-10-maleimidoethylacetamide (maleimidomonoamide-DOTA) with said polypeptide. In one embodiment, a chelating polypeptide encompassed by the present disclosure is obtained by reacting the DOTA derivative DOTAGA (2,2′,2″-(10-(2,6-dioxotetrahydro-2H-pyran-3-yl)-1,4,7,10-tetraazacyclododecane-1,4,7-triyl)triacetic acid) with said polypeptide. Additionally, 1,4,7-triazacyclononane-1,4,7-triacetic acid (NOTA) and derivatives thereof may be used as chelators. Hence, in one embodiment, a chelating environment of the HMGB1 binding polypeptide, HMGB1 binding polypeptide in heterodimeric form, fusion protein or conjugate is provided by NOTA or a derivative thereof. In one embodiment, a chelating polypeptide encompassed by the present disclosure is obtained by reacting the NOTA derivative NODAGA (2,2′-(7-(1-carboxy-4-((2,5-dioxopyrrolidin-1-yl)oxy)-4-oxobutyl)-1,4,7-triazonane-1,4-diyl)diacetic acid) with said polypeptide. The most commonly used acyclic polyaminopolycarboxylate chelators are different derivatives of DTPA (diethylenetriamine-pentaacetic acid). Hence, polypeptides having a chelating environment provided by diethylenetriaminepentaacetic acid or derivatives thereof are also encompassed by the present disclosure.

In further aspects of the present disclosure, there is provided a polynucleotide encoding an HMGB1 binding polypeptide or fusion protein as described herein; an expression vector comprising said polynucleotide; and a host cell comprising said expression vector.

Also encompassed by this disclosure is a method of producing HMGB1 binding polypeptide or fusion protein as described above, comprising culturing said host cell under conditions permissive of expression of said polypeptide from its expression vector, and isolating the polypeptide.

The HMGB1 binding polypeptide or fusion protein of the present disclosure may alternatively be produced by non-biological peptide synthesis using amino acids and/or amino acid derivatives having protected reactive side-chains, the non-biological peptide synthesis comprising
step-wise coupling of the amino acids and/or the amino acid derivatives to form a polypeptide or fusion protein as described herein having protected reactive side-chains,
removal of the protecting groups from the reactive side-chains of the polypeptide or fusion protein, and
folding of the polypeptide in aqueous solution.

A conjugate as disclosed herein may be produced by the conjugation of at least one HMGB1 binding polypeptide or fusion protein as described herein to at least one additional moiety. The skilled person is aware of conjugation methods, such as conventional chemical conjugation methods, for example using charged succinimidyl esters or carbodiimides.

It should be understood that the HMGB1 binding polypeptide according to the present disclosure may be useful as a therapeutic, diagnostic and/or prognostic agent in its own right or as a means for targeting other therapeutic or diagnostic agents, with e.g. direct or indirect effects on HMGB1. A direct therapeutic effect may for example be accomplished by antagonizing HMGB1 action. An indirect therapeutic effect may for example be accomplished by pretargeting using HMGB1 binding polypeptides as described above.

Thus, in another aspect, there is provided a composition comprising an HMGB1 binding polypeptide, fusion protein or conjugate as described herein and at least one pharmaceutically acceptable excipient or carrier. In one embodiment, said composition further comprises at least one additional active agent, such as at least two additional active agents, such as at least three additional active agents.

The small size and robustness of the HMGB1 binding polypeptides of the present disclosure confer several advantages over conventional monoclonal antibody based therapies. Such advantages include advantages in formulation, modes of administration, such as alternative routes of administration, administration at higher molar doses than antibodies and absence of Fc-mediated side effects. In particular when treating acute inflammatory conditions, the short plasma half-life of the polypeptides described herein is advantageous over monoclonal antibodies which have a long residence time. The agents of the present disclosure are contemplated for oral, topical, intravenous, intraperitoneal, subcutaneous, pulmonary, transdermal, intramuscular, intranasal, buccal, sublingual or suppository administration, such as for intravenous or subcutaneous administration.

In another aspect of the present disclosure, there is provided an HMGB1 binding polypeptide, fusion protein, conjugate or composition as described herein for use as a medicament, a prognostic agent and/or a diagnostic agent. In one embodiment, there is provided an HMGB1 binding polypeptide, fusion protein, conjugate or composition for use in the treatment, diagnosis or prognosis of an HMGB1 related disorder. In one embodiment, said use in diagnosis is carried out in vivo. In another embodiment, said use in diagnosis is carried out in vitro. In one embodiment, said use in prognosis is carried out in vivo. In another embodiment, said use in prognosis is carried out in vitro.

In one embodiment, said HMGB1 binding polypeptide, fusion protein, conjugate or composition is provided for use as a medicament. In a more specific embodiment, there is provided an HMGB1 binding polypeptide, fusion protein, conjugate or composition as described herein, for use as a medicament to modulate HMGB1 function, such as to modulate HMGB1 function in vivo. As used herein, the term “modulate” refers to changing the activity, such as partially inhibiting or fully inhibiting HMGB1 function.

In one embodiment, there is provided an HMGB1 binding polypeptide, fusion protein, conjugate or composition for use in the treatment of an HMGB1 related disorder.

In one embodiment, there is provided an HMGB1 binding polypeptide, fusion protein, conjugate or composition for use in the diagnosis of an HMGB1 related disorder. In a more specific embodiment, said use in diagnosis is carried out in vivo. In another specific embodiment, said use in diagnosis is carried out in vitro.

In one embodiment, there is provided an HMGB1 binding polypeptide, fusion protein, conjugate or composition for use in the prognosis of an HMGB1 related disorder. In a more specific embodiment, said use in prognosis is carried out in vivo. In another specific embodiment, said use in prognosis is carried out in vitro.

As used herein, the term “HMGB1 related disorder” refers to any disorder, disease or condition in which HMGB1 action plays a role. Examples of HMGB1 related disorder include inflammatory diseases, respiratory diseases, autoimmune diseases, infectious diseases, trauma, cardiovascular disease, neurodegenerative diseases, metabolic disorders, liver injury and cancers.

It is to be understood that said HMGB1 binding polypeptide, fusion protein, conjugate or composition may be used as the sole diagnostic or prognostic agent or as a companion diagnostic and/or prognostic agent.

In a related aspect, there is provided a method of treatment of an HMGB1 related disorder, comprising administering to a subject in need thereof an effective amount of an HMGB1 binding polypeptide, fusion protein, conjugate or composition as described herein. In a more specific embodiment of said method, the HMGB1 binding polypeptide, fusion protein, conjugate or composition as described herein modulates HMGB1 function in vivo. The skilled person will appreciate that any description in relation to the use of HMGB1 binding polypeptide, fusion protein, conjugate or composition as described herein for treatment of a disease or disorder is equally relevant for the related therapeutic method. For the sake of brevity, such description will not be repeated here.

In another aspect of the present disclosure, there is provided an in vitro method of detecting HMGB1, comprising providing a sample suspected to contain HMGB1, contacting said sample with an HMGB1 binding polypeptide, fusion protein, conjugate or composition as described herein, and detecting the binding of the HMGB1 binding polypeptide, fusion protein, conjugate or composition to indicate the presence of HMGB1 in the sample.

In one embodiment, said method further comprises an intermediate washing step for removing non-bound polypeptide, fusion protein, conjugate or composition, after contacting the sample.

In another aspect of the present disclosure, there is provided a diagnostic or prognostic method for determining the presence of HMGB1 in a subject, the method comprising the steps:
a) contacting the subject, or a sample isolated from the subject, with an HMGB1 binding polypeptide, fusion protein, conjugate or composition as described herein, and
b) obtaining a value corresponding to the amount of the HMGB1 binding polypeptide, fusion protein, conjugate or composition that has bound in said subject or to said sample.

In one embodiment, said method further comprises an intermediate washing step for removing non-bound polypeptide, fusion protein, conjugate or composition, after contacting the subject or sample and before obtaining a value.

In one embodiment, said method further comprises a step of comparing said value to a reference. Said reference may be by a numerical value, a threshold or a visual indicator, for example based on a color reaction. The skilled person will appreciate that different ways of comparison to a reference are known in the art and may be suitable for use.

In one embodiment of such a method, said subject is a mammalian subject, such as a human subject. In one embodiment, said method is performed in vivo. In another embodiment, said method is performed in vitro.

In one embodiment, the diagnostic or prognostic method is a method for medical imaging in vivo as discussed above. Such a method comprises the systemic administration of an HMGB1 binding entity as disclosed herein (i.e. the polypeptide per se, or the fusion protein, conjugate or composition containing it) to a mammalian subject. The HMGB1 binding entity is directly or indirectly labelled, with a label comprising a radionuclide suitable for medical imaging (see above for a list of contemplated radionuclides). Furthermore, the method for medical imaging comprises obtaining one or more images of at least a part of the subject's body using a medical imaging instrument, said image(s) indicating the presence of the radionuclide inside the body.

In certain embodiments of the various medical uses and methods of treatment, diagnosis and prognosis disclosed herein, said HMGB1 related disorder is selected from the group consisting of inflammatory diseases, respiratory diseases, autoimmune diseases, infectious diseases, trauma, cardiovascular disease, neurodegenerative diseases, metabolic disorders, liver injury and cancers.

Examples of specific indications within these categories, which are also embodiments of HMGB1 related disorders within the context of the present disclosure for which a polypeptide with affinity for HMGB1 is considered to have therapeutic, diagnostic or prognostic relevance, are: arthritis (such as rheumatoid arthritis, collagen-induced arthritis, crystal-induced arthritis ankylosing spondylitis), atherosclerosis, hepatitis, inflammatory bowel disease, chronic inflammatory anemia, myositis, pancreatitis, pulmonary fibrosis, pulmonary inflammation, hepatic ischemia-reperfusion injury, drug-induced liver intoxication (such as acetaminophen/paracetamol intoxication), acute or chronic liver failure, nonalcoholic fatty liver disease, liver fibrosis, cirrhosis, hemolytic uremic syndrome, systemic lupus erythematosus, cutaneous lupus erythematosus, lupus nephritis, glomerulonephritis, juvenile idiopathic arthritis, antineutrophilic cytoplasmatic antibody (ANCA)-associated vasculitis, systemic vasculitis scleroderma, Sjogren syndrome, Behcet's disease, cancer (such as breast cancer, colorectal cancer, hepatocellular carcinoma, lung cancer, pancreatic cancer, renal cell carcinoma, melanoma and mesothelioma), ischemia/reperfusion, stroke, ischemic brain injury, chemical toxemia, traumatic brain injury, neuroinflammation, epileptogenesis, cognitive dysfunctions, atherosclerosis, gastric ulcer, hyperoxia, sepsis, endotoxemia, hemorrhagic shock, cerebrovascular disease, myocardial infarction, heart failure, Alzheimer's disease, multiple sclerosis, epilepsy, diabetes, obesity, transplant rejection, chronic kidney disease, sciatica and neuropathic pain.

In a more specific embodiment of any one of the foregoing therapeutic aspects of the disclosure, the HMGB1 related disorder is selected from the group consisting of cerebrovascular diseases, chronic inflammatory anemia, acute liver failure, drug-induced liver intoxication, hemolytic uremic syndrome, systemic lupus erythematosus, mesothelioma, lung cancer, stroke, sepsis, sciatica and neuropathic pain, such as selected from the group consisting of drug-induced liver intoxication, sepsis, sciatica and neuropathic pain.

In a more specific embodiment of any one of the foregoing diagnostic or prognostic aspects of the disclosure, the HMGB1 related disorder is selected from the group consisting of traumatic brain injury, neuroinflammation, epileptogenesis, cognitive dysfunctions, stroke, rheumatoid arthritis, systemic lupus erythematosus, lupus nephritis, juvenile idiopathic arthritis, antineutrophilic cytoplasmatic antibody associated vasculitis, Behcet's disease, chronic inflammatory anemia, cancer (such as breast cancer, colorectal cancer, hepatocellular carcinoma, lung cancer, pancreatic cancer, melanoma and mesothelioma), hemolytic uremic syndrome, systemic inflammatory response syndrome, sepsis, drug-induced liver intoxication, sciatica and neuropathic pain, such as selected from the group consisting of traumatic brain injury, stroke, lung cancer, mesothelioma, hemolytic uremic syndrome, drug-induced liver intoxication, sepsis, sciatica and neuropathic pain.

More information concerning HMGB1 and its role as target for therapeutic intervention, or as biomarker for diagnosis and/or prognosis, is found in the reviews by Venereau et al (2016), supra, Andersson and Tracey (2011), supra, and Kang et al (2014) Molecular Aspects of Medicine 40:1-116.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1 is a listing of the amino acid sequences of examples of selected HMGB1 A-box binding polypeptides and alanine/serine mutated variants thereof (SEQ ID NO:1-29); HMGB1 B-box binding polypeptides (SEQ ID NO:30-32); control polypeptides with affinity for an irrelevant target (Z03638, SEQ ID NO:33 and Z04726, SEQ ID NO:54); the albumin binding polypeptide PP013 (SEQ ID NO:34); A-box binding polypeptides in fusion with PP013 (SEQ ID NO:35-36); B-box binding polypeptide in fusion with PP013 (SEQ ID NO:37); homodimeric A-box binding polypeptides in fusion with PP013 (SEQ ID NO:43-45); heterodimeric A-box and B-box binding polypeptides in fusion with PP013 (SEQ ID NO:38-42, SEQ ID NO:46-48 and SEQ ID NO:55); control polypeptide (homodimeric Z03638) in fusion with PP013 (SEQ ID NO:49); full-length rat HMGB1 (SEQ ID NO:50); the A-box domain of human HMGB1, boxA (SEQ ID NO:51); the A-box domain of human HMGB1 with an N-terminal GST-tag, GST-boxA (SEQ ID NO:52); and the B-box domain of human HMGB1 with an N-terminal GST-tag, GST-boxB (SEQ ID NO:53). Listed amino acid sequences were used in selection, screening and/or characterization of the binding polypeptides of the disclosure. The deduced HMGB1 A-box or B-box binding motifs (BM) extend from residue 8 to residue 36 in sequences with SEQ ID NO:1-14, 16, 20-23 and 27-32. The amino acid sequences of the 49 amino acid residues long polypeptides (BMod) predicted to constitute the complete three-helix bundle within each of these Z variants extend from residue 7 to residue 55.

FIG. 2 shows binding of four different HMGB1 variants to Z-ABD polypeptides measured by Biacore, as described in Example 3. HMGB1(disulfide) (FIGS. 2A and 2E), HMGB1(red) (FIGS. 2B and 2F), boxA(disulfide) (FIGS. 2C and 2G) and GST-boxA(red) (FIGS. 2D and 2H), respectively, were injected over ZA3812 (FIG. 2A-D) and ZA3914 (FIG. 2E-H), respectively, captured on immobilized HSA. In FIG. 2A-C and FIG. 2E-G, the injected concentrations were 10 nM (black broken line), 40 nM (black solid line), 160 nM (gray broken line) and 640 nM (gray solid line), i.e. of HMGB1(disulfide), HMGB1(red) and boxA(disulfide), respectively, whereas in FIG. 2D and FIG. 2H, the injected concentrations were 2.5 nM (black broken line), 10 nM (black solid line), 40 nM (gray broken line) and 160 nM (gray solid line), i.e. of GST-boxA(red).

FIG. 3 shows inhibition of HMGB1-induced cell migration by the A-box binding polypeptide ZH 01 (FIG. 3A) and by the B-box binding polypeptides ZH 31 (FIG. 3B) and ZH 30 (FIG. 3C), respectively. Z04726 and ZZA4014 are negative control variants. The results are presented as cells/field measured by the number of cells on the lower side of the filter counted with a Leica DM LS2 microscope.

FIG. 4 shows analysis by the cAMP Hunter eXpress CXCR4 CHO-K1 GPCR Assay. A: Analysis of luminescent signal after HMGB1-CXCL12 binding to CXCR4. HMGB1 (200 nM-0.3 nM) was added together with CXCL12 (0.4 nM) and inhibited the luminescent signal after 30 min stimulation. B: Analysis of luminescent signal after ZZA4263 inhibition of the HMGB1-CXCL12 complex binding to CXCR4. ZZA4263 (1000 nM-0.0006 nM) or PBS was added together with HMGB1 (10 nM) and CXCL12 (0.4 nM) and incubated with the cells for 30 min. ZZA4263 enabled the luminescent signal to return (black squares), whereas the PBS negative control did not show any effect on the luminescent signal (grey dots).

FIG. 5A shows the PK profiles of ZZA4263 in mice after i.v. (solid dots) and i.p. (open dots) administration, respectively. FIG. 5B shows the PK profile of ZZA4263 in rat after i.v. administration.

FIG. 6 shows the evaluation of ZZA4263 in a CLP-induced sepsis model in mice, depicted as the effect of ZZA4263 on the survival rate (FIG. 6A) and Murine Sepsis Score (FIG. 6B), respectively.

### EXAMPLES

### Summary

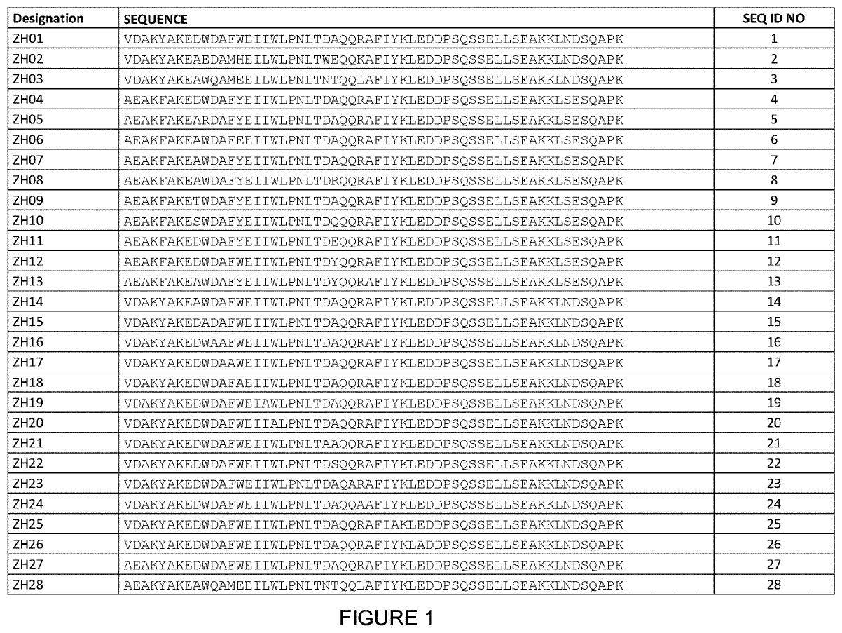

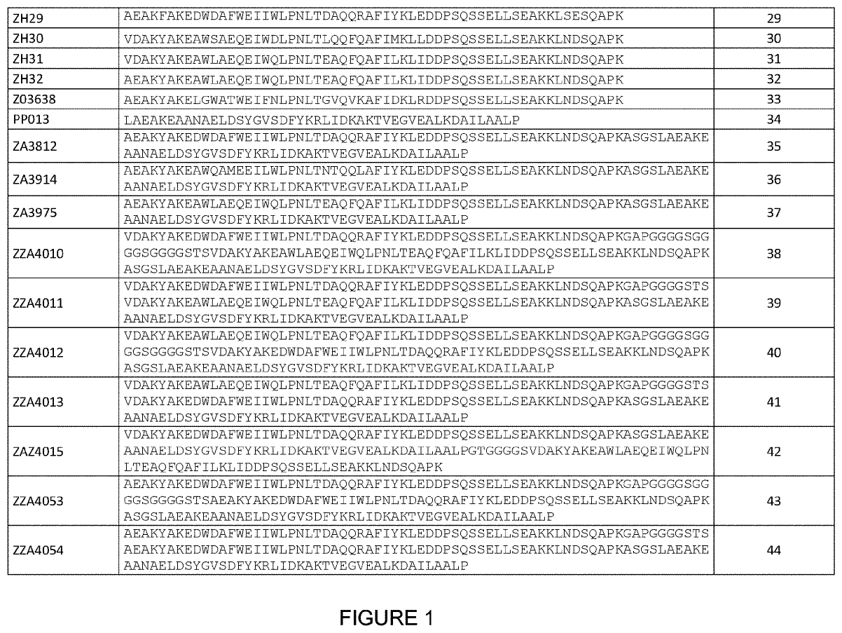

The following Examples disclose the development of novel Z variant molecules targeting the human High Mobility Group Box 1 protein (HMGB1), based on phage display technology. The polypeptides described herein were sequenced, and their amino acid sequences are listed in FIG. 1 with the sequence identifiers SEQ ID NO:1-32. The Examples further describe the characterization of HMGB1 binding polypeptides, and demonstrate their in vitro and in vivo functionality. Furthermore, a number of fusion protein constructs were prepared in the context of a “formatting program”, in order to optimize the potencies of said polypeptides. The Examples describe the characterization of these formatted HMGB1 binding polypeptides and demonstrate their in vitro and in vivo functionality.

Different full-length HMGB1 proteins and fragments thereof used in the Examples described below are listed in Table 3. All HMGB1 target proteins were produced by HMGBiotech Srl, Milano, Italy. The HMGB1 proteins used correspond to the amino acid sequence of rat HMGB1. However, rat and human HMGB1 are identical in sequence within the A-box and the B-box domains. The alternative denotations in Table 3 follow the systematic nomenclature proposed by Antoine et al (2014) Mol Med 20:135-137).

TABLE 3
HMGB1 proteins and fragments thereof
used in selection, screening and
characterization of HMGB1 binding polypeptides

| | SEQ | |
|---|---|---|
| | ID | |
| Designation | NO¹ | Description |
| HMGB1 | 50 | Fully reduced form of full-length |
| (red) | | HMGB1; HMGB1C23hC45hC106h |
| HMGB1 | 50 | Disulfide linked form of full-length |
| (disulfide) | | HMGB1; HMGB1C23-C45C106h |
| boxA | 51 | Disulfide linked form of A-box |
| (disulfide) | | domain; boxAC23-C45C106h |
| boxA(red) | 51 | Reduced form of A-box domain |
| GST-boxA | 52 | Reduced form of A-box domain |
| (red) | | with N-terminal GST tag |
| GST-boxB | 53 | Reduced form of B-box domain |
| (red) | | with N-terminal GST tag |
| b-HMGB1 | 50 | Biotinylated, fully reduced form |
| (red) | | of full-length HMGB1 |
| b-boxA | 51 | Biotinylated, disulfide-linked |
| (disulfide) | | form of A-box domain |
| b-GST-boxA | 52 | Biotinylated, reduced form of A-box |
| (red) | | domain with N-terminal GST tag |
| b-GST-boxB | 53 | Biotinylated, reduced form of B-box |
| (red) | | domain with N-terminal GST tag |

¹Unmodified amino acid sequence, independent of redox status

### Example 1

### Selection and Screening of HMGB1 Binding Z Variants

### Summary

In this Example, the reduced (i.e. thiol) forms of the A-box domain (boxA(red)) and the B-box domain (boxB(red)) of human HMGB1 were used as targets in phage display selections using a phage library of Z variants. Selected clones were DNA sequenced, produced in E. coli periplasmic fractions and assayed against full-length HMGB1, A-box or B-box in ELISA (enzyme-linked immunosorbent assay).

### Materials and Methods

Biotinylation of target protein: Glutathione S-transferase (GST) fused target proteins GST-boxA(red) (SEQ ID NO:52) and GST-boxB(red) (SEQ ID NO:53), as well as boxA(disulfide) (SEQ ID NO:51) and HMGB1(disulfide) (SEQ ID NO:50), were biotinylated using No-Weigh EZ-Link Sulfo-NHS-LC-Biotin (Thermo Scientific, cat. no. 21327) at a 10×molar excess, according to the manufacturer's recommendations. The reactions were performed at room temperature (RT) for 30-40 min. Buffer exchange to phosphate buffered saline (PBS; 10 mM phosphate, 137 mM NaCl, 2.68 mM KCl, pH 7.4) was performed both before and after biotinylation using PD-10 desalting columns (GE Healthcare, cat. no. 17-0851-01) according to the manufacturer's instructions.

Biotinylation of antibody: Anti-GST antibody (goat-polyclonal, Abcam, cat. no. ab6613) was biotinylated as described above.

Phage display selection of HMGB1 binding Z variants: A library of random variants of protein Z displayed on bacteriophage, constructed in phagemid pAY02592 essentially as described in Grönwall et al (2007) J Biotechnol 128:162-183, was used to select HMGB1 binding polypeptides. In this library, an albumin binding domain (“ABD”, i.e. domain GA3 of protein G from Streptococcus strain G148) is used as fusion partner to the Z variants. The library is denoted Zlib006Naive.II and has a size of 1.5×10¹⁰ library members (Z variants). Production of phage stock from phagemid library Zlib006Naive.II was essentially performed as described earlier (see e.g. PCT publication WO2017/072280) using E. coli RRIΔM15 cells (Rüther et al (1982) Nucleic Acids Res 10:5765-5772) from a glycerol stock of the library. The phage particles were precipitated in PEG/NaCl (polyethylene glycol/sodium chloride) from the supernatant twice, filtered and dissolved in PBS and glycerol as described in Grönwall et al supra. Phage stocks were stored at −80° C. before use.

Selections against GST-boxA(red), GST-boxB(red) and the corresponding biotinylated proteins, b-GST-boxA(red) and b-GST-boxB(red), were performed in four cycles. In tracks 1 and 2, Dynabeads® M-280 Streptavidin (SA beads, Invitrogen, cat. no. 11206D) were used to catch the b-GST-boxA(red):Z-variant and b-GST-boxB(red):Z-variant complexes. In tracks 3 and 4, SA beads coated with biotin-anti-GST antibody were used to catch the GST-boxA(red):Z-variant and GST-boxB(red):Z-variant complexes. As selection proceeded, the tracks were further divided according to target concentration and number and/or time of washes. Phage stock preparation and amplification of phage between selection cycles were performed essentially as described for selection against another biotinylated target in PCT publication WO2009/077175, with the following exceptions: 1) for amplification of phage particles between selection cycles 1 and 2, E. coli strain ER2738 (Lucigen, Middleton, Wis., USA) cells were grown to log phase in TSB supplemented with 10 μg/ml tetracycline and 2% glucose; 2) Infection was done during 30 min.

In order to reduce the amount of background binders, pre-selection was performed in each cycle using SA beads coated with biotin-GST, biotinylated as described previously for b-GST-boxA(red) and b-GST-boxB(red). Furthermore, in tracks 3 and 4, pre-selection was also performed using SA beads coated with biotin-anti-GST antibody. During pre-selection, the phage stock was incubated with coated beads end-over-end for 30-60 min at RT. All tubes and beads used in the pre-selections or selection were pre-blocked with PBS supplemented with 3% bovine serum albumin (BSA, Sigma, cat. no. A3059-100G) and 0.1% Tween20 (PBSTB).

Selection was performed in solution in PBS supplemented with 10% fetal bovine serum (FBS; Gibco, cat. no. 10108-165) and 0.1% Tween20 (Acros Organics, cat. no. 233362500) and 2 mM dithiothreitol (DTT; Acros Organics, cat. no. 165680250) at RT. The time for selection was approximately 120 min followed by wash with PBS supplemented with 0.1% Tween20 (PBST 0.1%) and catch of target-phage complexes on SA beads or SA beads coated with biotin-anti-GST antibody using 1 mg beads per 4-8 μg b-GST-boxA(red) and b-GST-boxB(red), respectively, and 0.4 μg GST-boxA(red) and GST-boxB(red), respectively.

In the final selection cycle, log phase bacteria were infected with eluate and diluted before spreading onto TBAB plates (30 g/l tryptose blood agar base, Oxoid, cat. no. CM0233B) supplemented with 0.2 g/l ampicillin in order to form single colonies to be used in ELISA screening.

An overview of the selection strategy, describing an increased stringency in successive cycles with a lowered target concentration and an increased number of washes, is shown in Table 4. Unless noted otherwise, washes were performed for 1 min using PBST 0.1%. Elution was carried out as described in WO2009/077175.

TABLE 4
Overview of the selection against HMGB1 using a primary library

| | | Phage stock | Proteins | | | | |
|---|---|---|---|---|---|---|---|
| | | from library | used in | | Target | Number | Duration |
| | Selection | or selection | pre- | Target | conc. | of | of last |
| Cycle | track | track | selection | protein | (nM) | washes | wash (h) |
| 1 | 1 | Zlib006Naive.II | b-GST | b-GST- | 100 | 2 | |
| | | | | boxA(red) | | | |
| 1 | 2 | Zlib006Naive.ll | b-GST | b-GST- | 100 | 2 | |
| | | | | boxB(red) | | | |
| 1 | 3 | Zlib006Naive.II | b-GST, b- | GST- | 100 | 2 | |
| | | | anti-GST | boxA(red) | | | |
| | | | antibody | | | | |
| 1 | 4 | Zlib006Naive.II | b-GST, b- | GST- | 100 | 2 | |
| | | | anti-GST | boxB(red) | | | |
| | | | antibody | | | | |
| 2 | 1-1 | 1 | b-GST | b-GST- | 50 | 4 | |
| | | | | boxA(red) | | | |
| 2 | 1-2 | 1 | b-GST | b-GST- | 10 | 4 | |
| | | | | boxA(red) | | | |
| 2 | 2-1 | 2 | b-GST | b-GST- | 50 | 4 | |
| | | | | boxB(red) | | | |
| 2 | 2-2 | 2 | b-GST | b-GST- | 10 | 4 | |
| | | | | boxB(red) | | | |
| 2 | 3-1 | 3 | b-GST, b- | GST- | 50 | 4 | |
| | | | anti-GST | boxA(red) | | | |
| 2 | 3-2 | 3 | b-GST, b- | GST- | 10 | 4 | |
| | | | anti-GST | boxA(red) | | | |
| 2 | 4-1 | 4 | b-GST, b- | GST- | 50 | 4 | |
| | | | anti-GST | boxB(red) | | | |
| 2 | 4-2 | 4 | b-GST, b- | GST- | 10 | 4 | |
| | | | anti-GST | boxB(red) | | | |
| 3 | 1-1-1 | 1-1 | b-GST | b-GST- | 25 | 6 | |
| | | | | boxA(red) | | | |
| 3 | 1-2-1 | 1-2 | b-GST | b-GST- | 1 | 6 | |
| | | | | boxA(red) | | | |
| 3 | 2-1-1 | 2-1 | b-GST | b-GST- | 25 | 6 | |
| | | | | boxB(red) | | | |
| 3 | 2-2-1 | 2-2 | b-GST | b-GST- | 1 | 6 | |
| | | | | boxB(red) | | | |
| 3 | 3-1-1 | 3-1 | b-GST, b- | GST- | 25 | 6 | |
| | | | anti-GST | boxA(red) | | | |
| 3 | 3-2-1 | 3-2 | b-GST, b- | GST- | 1 | 6 | |
| | | | anti-GST | boxA(red) | | | |
| 3 | 4-1-1 | 4-1 | b-GST, b- | GST- | 25 | 6 | |
| | | | anti-GST | boxB(red) | | | |
| 3 | 4-2-1 | 4-2 | b-GST, b- | GST- | 1 | 6 | |
| | | | anti-GST | boxB(red) | | | |
| 4 | 1-1-1-1 | 1-1-1 | b-GST | b-GST- | 12.5 | 10 | |
| | | | | boxA(red) | | | |
| 4 | 1-1-1-2 | 1-1-1 | b-GST | b-GST- | 2.5 | 30 | 1 |
| | | | | boxA(red) | | | |
| 4 | 1-2-1-1 | 1-2-1 | b-GST | b-GST- | 0.1 | 10 | |
| | | | | boxA(red) | | | |
| 4 | 1-2-1-2 | 1-2-1 | b-GST | b-GST- | 0.05 | 30 | 1 |
| | | | | boxA(red) | | | |
| 4 | 2-1-1-1 | 2-1-1 | b-GST | b-GST- | 12.5 | 10 | |
| | | | | boxB(red) | | | |
| 4 | 2-1-1-2 | 2-1-1 | b-GST | b-GST- | 2.5 | 30 | 1 |
| | | | | boxB(red) | | | |
| 4 | 2-2-1-1 | 2-2-1 | b-GST | b-GST- | 0.1 | 10 | |
| | | | | boxB(red) | | | |
| 4 | 2-2-1-2 | 2-2-1 | b-GST | b-GST- | 0.05 | 30 | 1 |
| | | | | boxB(red) | | | |
| 4 | 3-1-1-1 | 3-1-1 | b-GST, b- | GST- | 12.5 | 10 | |
| | | | anti-GST | boxA(red) | | | |
| 4 | 3-1-1-2 | 3-1-1 | b-GST, b- | GST- | 2.5 | 30 | 1 |
| | | | anti-GST | boxA(red) | | | |
| 4 | 3-2-1-1 | 3-2-1 | b-GST, b- | GST- | 0.1 | 10 | |
| | | | anti-GST | boxA(red) | | | |
| 4 | 3-2-1-2 | 3-2-1 | b-GST, b- | GST- | 0.05 | 30 | 1 |
| | | | anti-GST | boxA(red) | | | |
| 4 | 4-1-1-1 | 4-1-1 | b-GST, b- | GST- | 12.5 | 10 | |
| | | | anti-GST | boxB(red) | | | |
| 4 | 4-1-1-2 | 4-1-1 | b-GST, b- | GST- | 2.5 | 30 | 1 |
| | | | anti-GST | boxB(red) | | | |
| 4 | 4-2-1-1 | 4-2-1 | b-GST, b- | GST- | 0.1 | 10 | |
| | | | anti-GST | boxB(red) | | | |
| 4 | 4-2-1-2 | 4-2-1 | b-GST, b- | GST- | 0.05 | 30 | 1 |
| | | | anti-GST | boxB(red) | | | |

Production of Z variants for ELISA: The Z variants were produced and the periplasmic fraction of each individual variant was prepared as described in WO2017/072280. The final supernatant of the periplasmic extract contained the Z variants as fusions to ABD, expressed as AQHDEALE-[ZH ##]-VDYV-[ABD]-YVPG (Grönwall et al supra). ZH ## refers to individual, 58 amino acid residue Z variants.

Sequencing: In parallel with the ELISA screening, all clones were sequenced. PCR fragments were amplified from single colonies, sequenced and analyzed essentially as described in WO2009/077175

ELISA screening of Z variants against reduced A-box and B-box: The binding of Z variants to HMGB1 was analyzed in ELISA assays. Half-area 96-well ELISA plates (Greiner, cat. no. 675061) were coated at 4° C. overnight with 2 μg/ml of an anti-ABD goat antibody (produced in-house) diluted in coating buffer (50 mM sodium carbonate, pH 9.6; Sigma, cat. no. C3041). The antibody solution was poured off and the wells were washed in water and blocked with 100 μl of PBSC (PBS supplemented with 0.5% casein; Sigma, cat. no. C8654) for 1-3 h at RT. The blocking solution was discarded and 50 μl periplasmic solutions, diluted 1:16 with PBST 0.05%, were added to the wells and incubated for 1.5 to 2.5 h at RT under slow agitation. As a negative control, periplasmic ABD was added. The supernatants were poured off and the wells were washed 4 times with PBST 0.05%. Then, 50 μl of either b-GST-boxA(red), at a concentration of 2 nM, or b-GST-boxB(red), at a concentration of 100 nM, in PBSC+1-2 mM DTT, was added to each well of A-box binding Z-variants or B-box binding Z-variant, respectively. The plates were incubated for 1 h at RT followed by washes as described above. Streptavidin conjugated HRP (Thermo Scientific, cat. no. N100) diluted 1:30,000 in PBSC+1-2 mM DTT, was added to the wells and the plates were incubated for approximately 45 min. After washing as described above, 50 μl ImmunoPure TMB substrate (Thermo Scientific, cat. no. 34021) was added to the wells and the plates were treated according to the manufacturer's recommendations. The absorbance at 450 nm was measured using a multi-well plate reader, Victor3 (Perkin Elmer).

ELISA screening of Z variants against the disulfide form of A-box and against HMGB1: To find out if selected A-box binding Z variants could also bind the disulfide form of HMGB1, A-box binding variants were screened in the same setup as described above against b-boxA(disulfide) at a concentration of 1000 nM and b-HMGB1(disulfide) at a concentration of 100 nM in PBSC.).

ELISA analysis of selectivity for each HMGB1 domain: In a similar setup as described above, 100 nM of the HMGB1 domain not used as target in the particular selection track was used to identify unselective Z variants (i.e. b-GST-boxB(red) was used to screen Z-variants from b-GST-boxA(red) tracks and b-GST-boxA(red) was used to screen Z-variants from b-GST-boxB(red) tracks.

EC50 analysis of Z variants: A sub-group of HMGB1 binding Z variants was subjected to an analysis of their response against a dilution series of b-GST-boxA(red) or b-GST-boxB(red) following the procedure described above. The Z variants ZH 01 (SEQ ID NO:1), ZH 02 (SEQ ID NO:2) and ZH 03 (SEQ ID NO:3) were diluted 1:16 in PBST 0.05% and target protein b-GST-boxA(red) was diluted in PBSC (PBS with 0.5% casein (Sigma cat no. C-8654)) with 0.5-1 mM DTT and added at a concentration of 1 μM and diluted stepwise 1:5 down to 60 μM. The Z variants ZH 30 (SEQ ID NO:30) and ZH 31 (SEQ ID NO:31) were diluted 1:16 in PBST 0.05% and b-GST-boxB(red) was diluted in PBSC without DTT present and added at a concentration of 7 μM and diluted stepwise 1:5 down to 90 μM. As a background control, all Z variants were also assayed with no target protein added. Periplasm containing the ABD moiety only was used as a negative control. In the same assay, the selectivity of the Z variants was tested by incubating periplasm samples with the opposite HMGB1 domain (i.e. b-GST-boxA(red) or b-GST-boxB(red)) than the one used as target in selection, added at a concentration of 1000 nM. Data were analyzed using GraphPad Prism 5 and non-linear regression, and EC50 values (the half maximal effective concentration) were calculated.

### Results

Phage display selection of HMGB1 binding Z variants: Individual clones were obtained after four cycles of phage display selections against the reduced forms of the A-box and the B-box domains of HMGB1, i.e. boxA(red) and boxB(red), respectively.

Sequencing: Sequencing was performed for clones obtained after four cycles of selection. Each variant was given a unique identification number ##, and individual variants are referred to as ZH ##. The amino acid sequences of the 58 amino acid residues long Z variants are listed in FIG. 1 and in the sequence listing as SEQ ID NO:1-3 and 30-32. The deduced HMGB1 binding motifs extend from residue 8 to residue 36 in each sequence. The amino acid sequences of the 49 amino acid residues long polypeptides predicted to constitute the complete three-helix bundle within each of these Z variants extend from residue 7 to residue 55.

ELISA screening of Z variants against reduced A-box and B-box: The clones obtained after four cycles of selection were produced in 96-well plates and screened for b-GST-boxA(red) or b-GST-boxB(red) binding activity in ELISA. ZH 01, ZH 02 and ZH 03 were found to give an average response of 0.7, 0.2 and 0.6 AU, respectively (corresponding to at least 3× the blank control) against b-GST-boxA(red) at a target concentration of 2 nM. ZH 30 and ZH 31 were found to give responses of 0.14 and 0.47 AU, respectively (corresponding to at least 2.3×the blank control) against b-GST-boxB(red) at a concentration of 100 nM.

ELISA screening of Z variants against the disulfide form of A-box and against HMGB1: The A-box binding Z variants ZH 01, ZH 02, and ZH 03 were all found to bind to b-boxA(disulfide), giving responses of 3.06, 2.02 and 1.17 AU, respectively (corresponding to at least 3×the blank control) at a target concentration of 1000 nM. In the same way, ZH 01, ZH 02 and ZH 03 showed binding to b-HMGB1(disulfide) with a response of 3.07, 1.17 and 2.96 AU, respectively (corresponding to at least 3×the blank control) at a target concentration of 100 nM.

ELISA analysis of selectivity for each HMGB1 domain: No significant binding was detected to either biotin, GST or the HMGB1 domain not used as target in selection. These results indicate that the selected Z variants are specific to boxA(red) or boxB(red), respectively.

EC50 analysis of Z variants: A subset of Z variants having the highest ELISA values in the ELISA screening experiments described above was selected and subjected to a target titration in ELISA format. Periplasm samples were incubated with a serial dilution of either b-GST-boxA(red) or b-GST-boxB(red). Obtained values were analyzed and EC50 values calculated (Table 5).

TABLE 5
Calculated EC50 values from ELISA titration analysis

| Designation | SEQ ID NO | Target | EC50 (M) |
|---|---|---|---|
| ZH01 | 1 | boxA(red) | 2.2 × 10⁻⁹ |
| ZH02 | 3 | boxA(red) | 5.6 × 10⁻⁹ |
| ZH03 | 2 | boxA(red) | 2.7 × 10⁻⁹ |
| ZH30 | 30 | boxB(red) | 6.9 × 10⁻⁹ |
| ZH31 | 31 | boxB(red) | 9.9 × 10⁻⁹ |

### Example 2

### Production of Monomeric HMGB1 Binding Z Variants

### Summary

This Example describes the general procedure for subcloning and production of His-tagged Z variants and Z variants in fusion with an albumin binding domain, which are used throughout the characterization experiments that follow.

### Materials and Methods

Subcloning of Z variants with a His₆-tag: DNA encoding each Z variant was amplified from the library vector pAY02592. A subcloning strategy for construction of monomeric Z variant molecules with an N-terminal His₆-tag was applied using standard molecular biology techniques and essentially as described in PCT publication WO2009/077175. The Z gene fragments were subcloned into an expression vector, resulting in the encoded sequence MGSSHHHHHHLQ-[ZH ##]-VD.

Subcloning of Z variants in fusion with ABD: The N-terminal amino acids in positions 1 and 2 (V and D, respectively) of the Z variants ZH 01, ZH 03 and ZH 31 were mutated to the amino acid residues A and E, respectively, using standard molecular biology techniques. The resulting new Z variants, ZH 27 (SEQ ID NO:27), ZH 28 (SEQ ID NO:28) and ZH 32 (SEQ ID NO:32), were subcloned into an expression vector containing the ABD variant PP013 (SEQ ID NO:34). The constructs encoded by the expression vectors were in the format [ZH ##]-ASGS-PP013 and denoted ZA3812 (SEQ ID NO:35), ZA3914 (SEQ ID NO:36) and ZA3975 (SEQ ID NO:37).

Expression of HMGB1 binding Z variants: Production of Z variants was accomplished essentially as follows: E. coli T7E2 cells (GeneBridges) were transformed with plasmids containing the gene fragment of each respective HMGB1 binding Z variant and cultivated at 37° C. in 980 ml of TSB-YE medium supplemented with 50 μg/ml kanamycin. In order to induce protein expression, isopropyl-β-D-1-thiogalactopyranoside (IPTG) was added to a final concentration of 0.2 mM at OD₆₀₀=2 and the cultivation was incubated at 37° C. for another 5 h. The cells were harvested by centrifugation.

Purification of HMGB1 binding Z variants with a His₆-tag: Approximately 1-2 g of each cell pellet was re-suspended in binding buffer (20 mM sodium phosphate, 0.5 M NaCl, 20 mM imidazole, pH 7.4) supplemented with Benzonase® (Merck, cat. no. 1.01654.0001). After cell disruption, cell debris was removed by centrifugation and each supernatant was applied on a 1 ml His GraviTrap IMAC column (GE Healthcare, cat. no. 11-0033-99). Contaminants were removed by washing with wash buffer (20 mM sodium phosphate, 0.5 M NaCl, 60 mM imidazole, pH 7.4) and the Z variants were subsequently eluted with elution buffer (20 mM sodium phosphate, 0.5 M NaCl, 500 mM imidazole, pH 7.4). Z variants were subjected to a second purification step by reverse phase chromatography (RPC), for which each Z variant was loaded onto a 1 ml Resource 15RPC column (GE Healthcare), previously equilibrated with RPC solvent A (0.1% trifluoroacetic acid (TFA), 10% acetonitrile (ACN), 90% water). After column wash with RPC solvent A, bound proteins were eluted with a linear gradient of 0-60% RPC solvent B (0.1% TFA, 80% ACN, 20% water) for 18 ml. The buffer was then exchanged to DPBS (Corning, cat. no. 21-031-CVR) using PD-10 desalting columns.

Protein concentrations were determined by measuring the absorbance at 280 nm, using a NanoDrop® ND-1000 spectrophotometer (Saveen Werner AB) and the extinction coefficient of the respective protein. In batches where endotoxin levels exceeded 10 EU/mg, an endotoxin removal step was applied using a 1 ml EndoTrap red column (Hyglos, cat. no. 321063) according to supplier's recommendations. Samples with a concentration less than approximately 1 mg/ml were concentrated using Am icon Ultra-4, Ultracel-3K (Merck Millipore). The purity was analyzed by SDS-PAGE stained with Coomassie Blue, endotoxin level was analyzed with Endosafe PTS (Charles River) and the identity of each purified Z variant was confirmed using LC/MS analysis.

Purification of HMGB1 binding Z variants in fusion with ABD: Approximately 5 g of each cell pellet was re-suspended in TST-buffer (25 mM Tris-HCl, 1 mM EDTA, 200 mM NaCl, 0.05% Tween20, pH 8.0) supplemented with Benzonase® (Merck). After cell disruption, clarification by centrifugation and passing through a 0.45 μm filter, each supernatant was applied on a gravity flow column with 5 ml agarose immobilized with an anti-ABD ligand (produced in-house). After washing with TST-buffer and 5 mM NH₄Ac pH 5.5 buffer, the ABD fused Z variants were eluted with 0.1 M HAc. The eluates were then subjected to a second purification step by RPC. 10% ACN was added to each eluate before loaded on a 3 ml Resource RPC column (GE Healthcare, cat. no. 17-1182-01) equilibrated with solvent A (10% ACN, 0.1% TFA, 90% milli-Q-water) and eluted using a gradient of 0-60% solvent B (80% ACN, 0.1% TFA, 20% milli-Q-water) for 54 ml. Eluted fractions were analyzed by SDS-PAGE and HPLC-MS and pooled. The buffer of the eluate was exchanged to DPBS using PD-10 desalting columns.

Endotoxin removal and determination of protein concentrations, as well as analysis of purity and identity, was performed as described above for Z variants with a His₆-tag.

### Results

Production of HMGB1 binding Z variants: The HMGB1 binding Z variants with His₆-tag or in fusion with ABD were successfully cloned and expressed as soluble gene products in E. coli. Each DNA construct was verified by DNA sequencing. SDS-PAGE analysis of each final protein preparation showed that these predominantly contained the HMGB1 binding Z variant. The correct identity and molecular weight of each Z variant were confirmed by HPLC-MS analysis.

### Example 3

### Characterization of Primary HMGB1 Binding Z Variants

### SUMMARY

In this Example, Z variants were characterized in vitro in terms of secondary structure, stability, binding profile and functional properties.

Biacore was used to characterize the interactions of the Z variants with full-length HMGB1 as well as with the individual A-box and B-box domains separately. The melting temperature and secondary structure content were analyzed by circular dichroism (CD) spectroscopy. Furthermore, the ability of Z variants to functionally block HMGB1-induced mechanisms was investigated using an in vitro cell migration assay.

### Materials and Methods

Circular dichroism (CD) spectroscopy analysis: The His₆-Z variants produced in Example 2 were diluted to 25 μM in PBS or H₂O. A CD spectrum at 250-195 nm was obtained at 20° C. In addition, a variable temperature measurement (VTM) was performed to determine the melting temperature (Tm). In the VTM, absorbance was measured at 221 nm while the temperature was raised from 20° C. to 80° C. (in some cases to 90° C.), with a temperature slope of 5° C./min. A new CD spectrum was obtained at 20° C. after the heating procedure, in order to study the refolding ability of the Z variants. The CD measurements were performed on a Jasco J-810 spectropolarimeter (Jasco Scandinavia AB) using a cell with an optical path length of 1 mm.

Biacore kinetic and specificity analysis: Kinetic constants (kₐ and k_{d}) and affinities (K_{D}) for HMGB1(red), HMGB1(disulfide), boxA(disulfide) and GST-boxA(red) were determined for the A-box binding Z-ABD polypeptides ZA3812 (SEQ ID NO:35) and ZA3914 (SEQ ID NO:36) (see Example 2), as well as for the HMGB1(red) interaction with the B-box binding polypeptide ZA3975, using a Biacore 2000 instrument (GE Healthcare).

The experiment was performed as a capture assay, in which human serum albumin (HSA, Novozymes, cat. no. 230-005) was immobilized on the carboxylated dextran layer of one CM5 chip surface (GE Healthcare, cat. no. BR100012) to immobilization levels of approximately 3000 RU. The immobilization was performed using amine coupling chemistry according to the manufacturer's protocol and using HBS-EP (GE Healthcare, cat. no. BR100188) as running buffer. In a first set of experiments, Z-ABD polypeptides were analyzed under non-reducing conditions. One flow cell surface was activated and deactivated for use as blank during ligand and analyte injections. In the kinetic experiment, HBS-EP was used as running buffer and the flow rate was 30 μl/min. The ligand, i.e. the Z-ABD polypeptide, was diluted in HBS-EP buffer to a concentration of 30 nM and injected during 2 min. The interaction between HSA and ABD had a very slow off rate, resulting in very low dissociation during the following measurement.

The analyte, i.e. HMGB1(disulfide) or boxA(disulfide), was diluted in HBS-EP buffer within a concentration range of 2.5 to 640 nM and injected during 6 min, followed by dissociation in running buffer for 12 min. After dissociation, the surfaces were regenerated with one injection of 10 mM HCl.

In a second set of experiments, ZA3812 and ZA3914 were analyzed under reducing conditions where 2 mM DTT was included in the sample and running buffer. Analyses were performed as described above, except that HMGB1(red) and GST-boxA(red) were used as targets.

In a third experiment, ZA3975 was analyzed for binding to HMGB1(red) under reduced conditions (1.5 mM DTT included in the sample and running buffer). The ligand ZA3975 was diluted in HBS-EP buffer to a concentration of 100 nM and injected during 4 min. The analyte, HMGB1(red), was diluted in HBS-EP buffer to a concentration within the range of 8.3 to 675 nM and injected during 4 min, followed by dissociation in running buffer for 20 min. After dissociation, the surfaces were regenerated with three injections of 10 mM HCl.

Kinetic constants were calculated from sensorgrams using the BiaEvaluation software 4.1 (GE Healthcare). The Langmuir 1:1 model was used for fitting of all data except for HMGB1(disulfide), where a model for heterogeneous ligand parallel reactions better represented the experimental data and bulk was set to zero. Double referencing was applied to all data prior to kinetic fit, i.e. responses obtained from a blank surface and from a blank cycle where buffer was injected instead of analyte were subtracted.

In vitro cell migration assay: Mouse 3T3 fibroblast cells were used to assess the ability of His₆-tagged Z variants to inhibit migration of cells in response to HMGB1 in modified Boyden Chambers (Neuroprobe Inc.). Mouse 3T3 fibroblasts were grown in Dulbecco's Modified Eagle Medium (DMEM, Gibco) with 10% fetal bovine serum (FBS, Gibco), 1% L-glutamine and antibiotics (Lonza). Prior to use, cells were centrifuged and the pellet was resuspended in DMEM (without FBS) without further washing. Polycarbonate filters (Neuroprobe Inc.; #PFA8) with 8 μm pores were coated with human fibronectin (Roche). Serum-free DMEM (negative control), DMEM containing 1 nM HMGB1(red) (positive control), and DMEM containing 1 nM HMGB1(red) plus Z variants ZH 01 (SEQ ID NO:1) and ZH 31 (SEQ ID NO:31) at a concentration of 10 or 100 ng/ml, were placed in the lower wells. A control polypeptide, Z04726 (SEQ ID NO:54), binding an irrelevant target, was used as negative control. boxA(red) (SEQ ID NO:51), a known competitor of HMGB1, was used at 1 nM concentration as positive control for inhibition of HMGB1 induced cell migration. Fibronectin-coated filters were placed so as to separate the lower and the upper wells, and 50000 3T3 cells were placed in the upper wells. The chambers were kept at 37° C. in a 5% CO₂, humidified incubator for 3 h. The cells were then fixed with 100% ethanol (Sigma) and stained 20 minutes with Giemsa Stain (Sigma). The cells on the lower side of the filter were counted with a Leica DM LS2 microscope at 400× magnification.

ZH 30 was tested in a migration assay by performing an extended titration to allow IC50 calculations with 0.2 nM HMGB1(red) and lower concentrations of Z variants (0.1 ng/ml, 0.3 ng/ml, 1 ng/ml). A control polypeptide, ZZA4014 (SEQ ID NO:49), binding an irrelevant target and cloned and produced as described in Example 4, was used as negative control in this set up.

### Results

CD analysis: The CD spectra determined for five HMGB1 binding Z variants with a His₆ tag showed that all variants have an α-helical structure at 20° C. as judged from the typical minima at 208 and 222 nm. Reversible folding was seen for all Z variants when spectra measured before and after heating to 80-90° C. were superimposed. The melting temperatures (Tm) are summarized in Table 6.

TABLE 6
Melting temperatures (Tm)

| Z variant | SEQ ID NO | Tm (° C.) |
|---|---|---|
| ZH01 | 1 | 49 |
| ZH02 | 2 | 58 |
| ZH03 | 3 | 54 |
| ZH30 | 30 | 53 |
| ZH31 | 31 | 57 |

Biacore kinetic and specificity analysis: The interactions of the A-box binding Z-ABD polypeptides ZA3812 (SEQ ID NO:35) and ZA3914 (SEQ ID NO:36) with four different HMGB1 variants were analyzed in a Biacore instrument by injecting various concentrations of HMGB1 variants over a surface containing ZA3812 and ZA3914, respectively, captured on immobilized HSA. Both ZA3812 and ZA3914 showed binding to all four tested HMGB1 variants. In addition, the interaction of the B-box binding Z-ABD polypeptide ZA3975 (SEQ ID NO:37) was analyzed for binding to HMGB1(red) in the same manner.

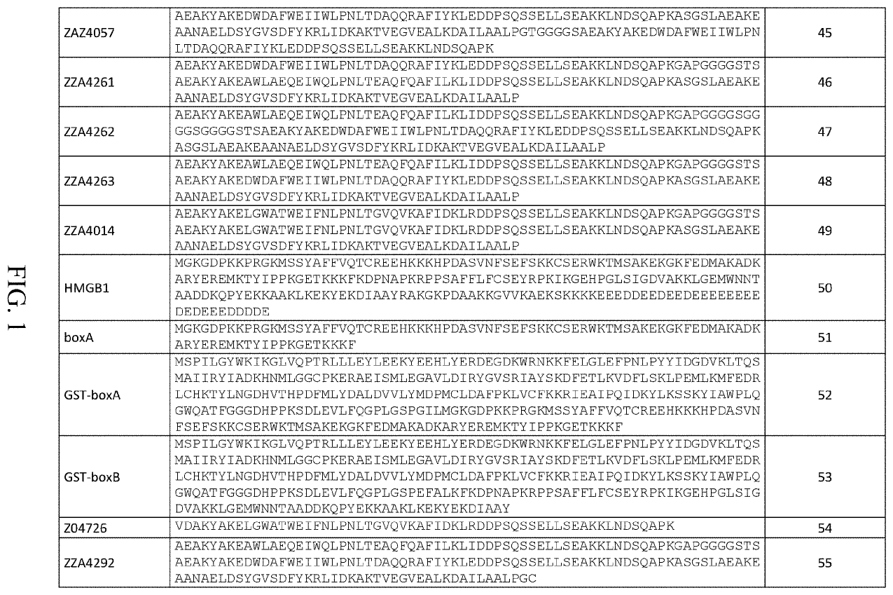

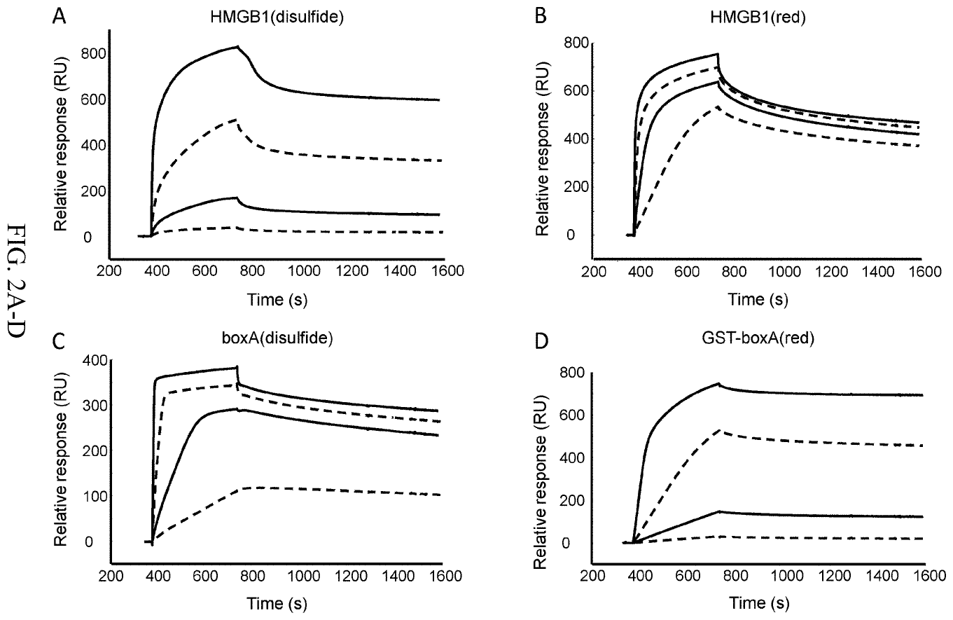

A summary of the estimated (based on data from four concentrations within the range 2.5 to 640 nM of injected HMGB1 variant for ZA3812 and ZA3914; or from two concentrations, 8.3 and 25 nM, for ZA3975) kinetic parameters (kₐ and k_{d}) and affinity constants (K_{D}) for binding of ZA3812, ZA3914 and ZA3975 to HMGB1 variants, obtained using a 1:1 interaction model or a heterogeneous ligand parallel reactions model, is given in Table 7. Resulting sensorgrams for the A-box binding polypeptides are displayed in FIGS. 2A and 2B.

TABLE 7
Kinetic parameters and affinity constants

| Z-ABD | | kₐ₁ | k_{d1} | kₐ₂ | k_{d2} | K_{D1} | K_{D2} |
|---|---|---|---|---|---|---|---|
| variant | Analyte | (1/Ms) | (1/s) | (1/Ms) | (1/s) | (M) | (M) |
| ZA3812 | HMGB1(disulfide) | 1.6 × 10⁵ | 1.8 × 10⁻² | 1.3 × 10⁴ | 3.5 × 10⁻⁵ | 1.1 × 10⁻⁷ | 2.7 × 10⁻⁹ |
| ZA3812 | boxA(disulfide) | 1.5 × 10⁵ | 2.1 × 10⁴ | | | 1.4 × 10⁻⁹ | |
| ZA3812 | HMGB1(red) | 5.0 × 10⁵ | 4.0 × 10⁻⁴ | | | 8.1 × 10⁻¹⁰ | |
| ZA3812 | GST-boxA(red) | 7.2 × 10⁴ | 7.6 × 10⁻⁵ | | | 1.1 × 10⁻⁹ | |
| ZA3914 | HMGB1(disulfide) | 1.8 × 10⁵ | 2.5 × 10⁻² | 1.4 × 10⁴ | 9.5 × 10⁻⁵ | 1.4 × 10⁻⁷ | 6.8 × 10⁻⁹ |
| ZA3914 | boxA(disulfide) | 1.7 × 10⁵ | 1.5 × 10⁻⁴ | | | 0.9 × 10⁻⁹ | |
| ZA3914 | HMGB1(red) | 1.8 × 10⁵ | 1.1 × 10⁻³ | | | 6.1 × 10⁻⁹ | |
| ZA3914 | GST-boxA(red) | 1.2 × 10⁵ | 1.0 × 10⁻⁴ | | | 0.8 × 10⁻⁹ | |
| ZA3975 | HMGB1(red) | 2.5 × 10⁶ | 2.8 × 10⁻⁴ | | | 1.1 × 10⁻¹⁰ | |

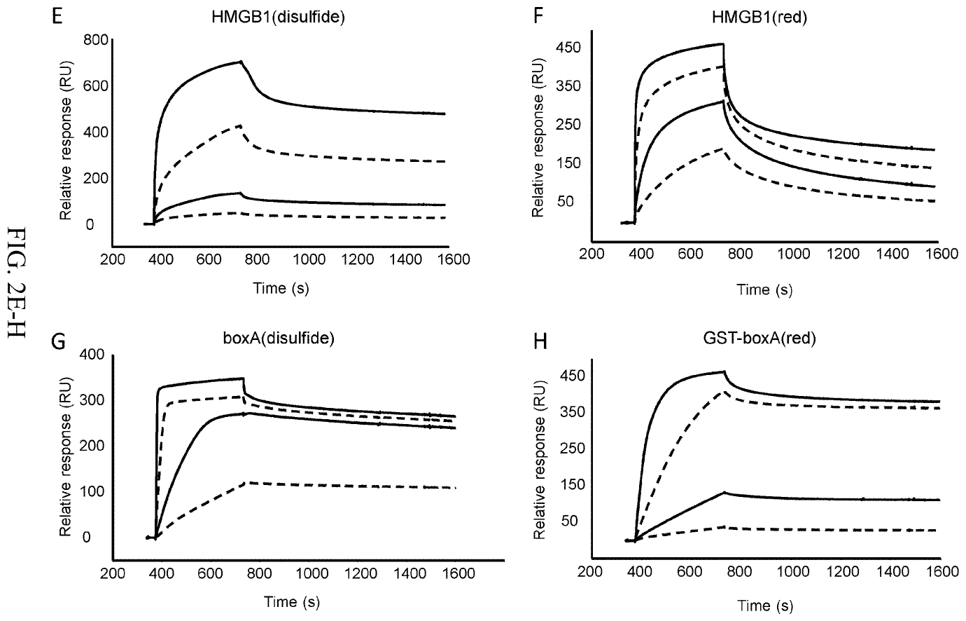

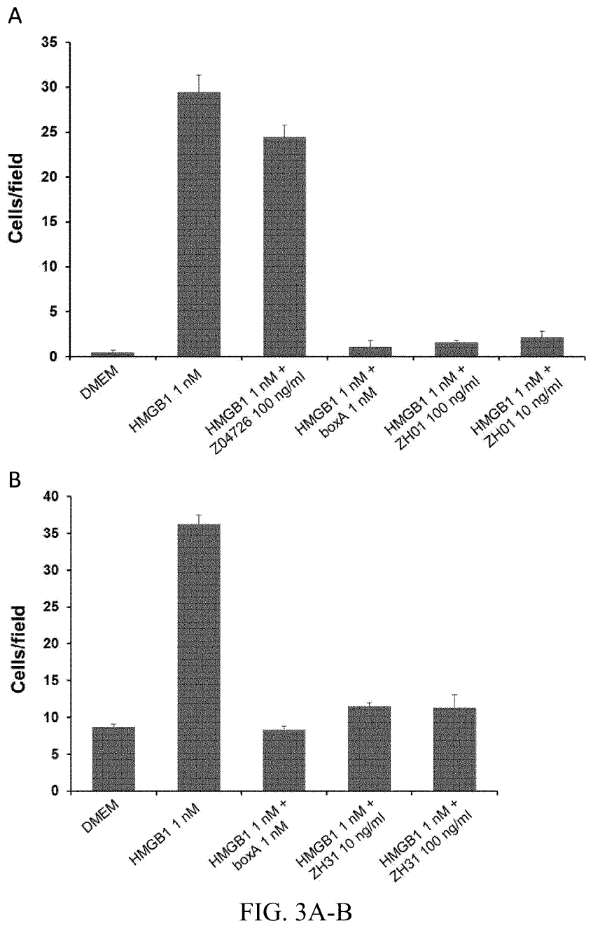

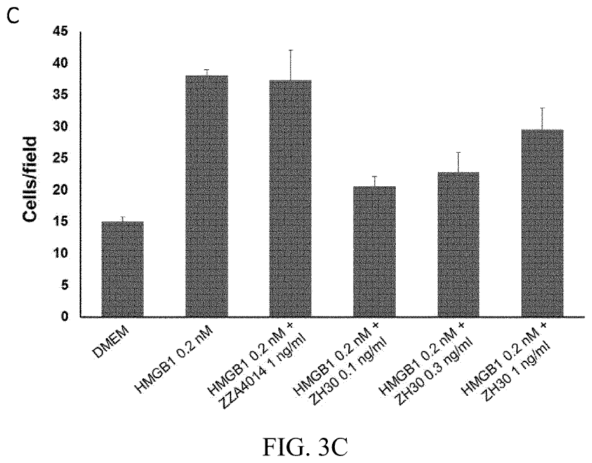

In vitro cell migration assay: ZH 01, binding to HMGB1 A-box, as well as ZH 31 and ZH 30, both binding to HMGB1 B-box, were shown to inhibit HMGB1 induced cell migration, whereas no inhibition was seen for the negative control Z variants Z04726 and ZZA4014 (FIG. 3A-3C).

### Example 4

### Design and Production of Homo- and Heterodimers in Fusion with ABD

### Summary

Five different heterodimeric polypeptides were designed, in which the A-box binding Z variant ZH 01 (SEQ ID NO:1) and the B-box binding Z variant ZH 31 (SEQ ID NO:31) were combined into a trispecific polypeptide targeting two different domains of HMGB1 and albumin. The order of Z variants with respect to one another and to ABD was varied. Different linkers were also evaluated. Furthermore, three homodimeric polypeptides based on ZH 01 were constructed. This Example describes the general procedure for subcloning and production of the formatted homo- and heterodimeric polypeptides in fusion with ABD, which are used throughout the characterization experiments that follow.

### Materials and Methods

Subcloning of formatted polypeptides in fusion with ABD: The N-terminus of respective Z variant was cloned with either VD or AE in amino acid positions 1 and 2, as described previously for monomeric Z variants. The subcloning of the homo- and heterodimeric constructs was performed using standard molecular biology techniques. The Z variants were subcloned into an expression vector containing DNA encoding the ABD variant PP013 (SEQ ID NO:34), The constructs encoded by the expression vectors were [ZH ##]-GAP(G₄S)₃TS-[ZH ##]-ASGS-PP013, [ZH ##]-GAP(G₄S)TS-[ZH ##]-ASGS-PP013 and [ZH ##]-ASGS-PP013-GT(G₄S)-[ZH ##], where the linkers GAP(G₄S)₃TS, GAP(G₄S)TS and GT(G₄S) are referred to in the following as L1, L2 and L3, respectively. An overview of the designed homo- and heterodimeric polypeptides is shown in Table 8.

TABLE 8
Homo- and heterodimeric polypeptides

| | SEQ | A-box | B-box | |
|---|---|---|---|---|
| | ID | binding | binding | |
| Designation | NO | Z | Z | Format |
| ZZA4010 | 38 | ZH01 | ZH31 | Z(boxA)-L1-Z(boxB)-ABD |
| ZZA4011 | 39 | ZH01 | ZH31 | Z(boxA)-L2-Z(boxB)-ABD |
| ZZA4012 | 40 | ZH01 | ZH31 | Z(boxB)-L1-Z(boxA)-ABD |
| ZZA4013 | 41 | ZH01 | ZH31 | Z(boxB)-L2-Z(boxA)-ABD |
| ZAZ4015 | 42 | ZH01 | ZH31 | Z(boxA)-ABD-L3-Z(boxB) |
| ZZA4053 | 43 | ZH27 | — | Z(boxA)-L1-Z(boxA)-ABD |
| ZZA4054 | 44 | ZH27 | — | Z(boxA)-L2-Z(boxA)-ABD |
| ZAZ4057 | 45 | ZH27 | — | Z(boxA)-ABD-L3-Z(boxB) |
| ZZA4261 | 46 | ZH27 | ZH32 | Z(boxA)-L2-Z(boxB)-ABD |
| ZZA4262 | 47 | ZH27 | ZH32 | Z(boxB)-L1-Z(boxA)-ABD |
| ZZA4263 | 48 | ZH27 | ZH32 | Z(boxB)-L2-Z(boxA)-ABD |
| ZZA4014¹ | 49 | — | — | Z03638-L2-Z3638-ABD |

¹negative control

Production of homo- and heterodimeric polypeptides: E. coli T7E2 cells were transformed with plasmids containing the gene fragment of each respective homo- and heterodimeric polypeptide. The cultivation and purification were carried out essentially as described for Z-ABD polypeptides in Example 2.

### Results

Production of homo- and heterodimeric polypeptides: The polypeptides were successfully cloned and expressed as soluble gene products in E. coli. Each DNA construct was verified by DNA sequencing. SDS-PAGE analysis of each final protein preparation showed that these predominantly contained the expected polypeptide. The correct identity and molecular weight of each polypeptide were confirmed by HPLC-MS analysis.

### Example 5

### Characterization of Homo- and Heterodimeric Polypeptides

### Summary

In this Example, the homo- and heterodimeric polypeptides produced as described in Example 4 were characterized in vitro in terms of binding profile and functional properties. The profile of the homo- and heterodimeric polypeptides' binding to HMGB1 was analyzed using an A200® biosensor (Attana, Stockholm, Sweden). Furthermore, the ability of the polypeptides to functionally block HMGB1-induced mechanisms was investigated using the in vitro cell migration assay described for Z-ABD polypeptides in Example 3.

### Materials and Methods

Binding analysis: The Attana A200® biosensor was used to characterize interactions between the dimeric polypeptides (SEQ ID NO:38-48) and HMGB1(red) under reducing conditions and/or HMGB1(disulfide) under non-reducing conditions. The monomeric ZA3812 (SEQ ID NO:35) was included for comparison. The experiment was performed as a capture assay, in which HSA was immobilized on Attana's sensor chip Low-NonSpecific-Binding (LNB). Immobilization was performed using amine coupling. The instrument has two sensor surfaces and HSA was immobilized on both surfaces. Around 110 Hz of HSA was stably immobilized on the surfaces.

In the kinetic experiment, HBS-EP+/−2 mM DTT was used as running buffer and the flow rate was 25 μl/min at 22° C. In each cycle, one polypeptide was tested against one concentration of HMGB1. The ligand, i.e. the HMGB1 binding polypeptide, was diluted in HBS-EP (+/−DTT) buffer to a concentration of 0.5 μg/ml and injected for 84 s over the HSA surface in channel A. The interaction between HSA and ABD had a very slow off-rate, resulting in a very low dissociation during the measurement. The analyte HMGB1(reduced) was diluted in HBS-EP+2 mM DTT within a concentration range of 1.33 to 21.4 μg/ml, and the analyte HMGB1(disulfide) was diluted in HBS-EP buffer within a concentration range of 0.62 to 10 μg/ml. Both analytes were subsequently injected for 84 s over both channel A and B, where channel B was used as a blank surface for curve subtraction. Buffer injections were also made for surface subtractions. After dissociation, the surfaces were regenerated with a 30 s injection of 100 mM HCl followed by a 30 s injection of 20 mM NaOH. Data was collected by Attester Software and subsequently processed in the Evaluation Software. To calculate the kinetic parameters (kₐ and k_{d}) and affinity constant (K_{D}), the experimental data were fitted using a 1:1 or a 1:2 binding model.

In vitro cell migration assay: The migration assay was performed essentially as described for monomeric Z variants in Example 3. One monomeric Z-ABD polypeptide, ZA3812 (SEQ ID NO:35); five heterodimeric polypeptides, ZZA4010 (SEQ ID NO:38), ZZA4011 (SEQ ID NO:39), ZZA4012 (SEQ ID NO:40), ZZA4013 (SEQ ID NO:41) and ZAZ4015 (SEQ ID NO:42); and three homodimeric polypeptides, ZZA4053 (SEQ ID NO:43), ZZA4054 (SEQ ID NO:44) and AZ4057 (SEQ ID NO:45), were tested in a migration assay by performing an extended titration, to allow IC50 calculations with 0.2 nM HMGB1 and reducing doses of polypeptides (0.1 nM, 0.3 nM, 1 nM). ZZA4014 (SEQ ID NO:49), binding an irrelevant target, was included as a negative control.

### Results

Binding analysis of dimeric polypeptides targeting HMGB1: The interactions of a set of dimeric polypeptides, as well as one monomeric polypeptide, with HMGB1(reduced) and/or HMGB1(disulfide) were analyzed using an Attana A200® biosensor instrument by injecting various concentrations of HMGB1 over a surface containing captured polypeptides in fusion with ABD bound by immobilized HSA. All tested polypeptides showed binding to both HMGB1 redox forms. A summary of the approximate (based on data from five concentrations within the range 0.62 to 21 nM, of injected HMGB1) kinetic parameters (kₐ and k_{d}) and affinity constant (K_{D}), obtained by fitting the experimental data using a 1:1 or a 1:2 binding model, are given in Table 9.

TABLE 9
Summary of kinetic parameters of HMGB1-binding polypeptides

| | | | Average | k_{d}¹ | Average Kp |
|---|---|---|---|---|---|
| | SEQ | k_{d}¹ | K_{D} | HMGB1 | HMGB1 |
| | ID | HMGB1 | HMGB1 | (disulfide) | (disulfide) |
| Designation | NO | (red) (s⁻¹) | (red) (M) | (s⁻¹) | (M) |
| ZA3812 | 35 | 1.3 × 10⁻² | 5.1 × 10⁻⁸ | 4.8 × 10⁻⁴ | 1.3 × 10⁻⁷ |
| ZZA4010 | 38 | 5.5 × 10⁻⁴ | 4.4 × 10⁻⁹ | 1.0 × 10⁻³ | 7.0 × 10⁻⁸ |
| ZZA4011 | 39 | 4.9 × 10⁻⁴ | 4.2 × 10⁻⁹ | 1.1 × 10⁻³ | 1.1 × 10⁻⁷ |
| ZZA4012 | 40 | 4.6 × 10⁻⁴ | 3.0 × 10⁻⁹ | 4.5 × 10⁻⁴ | 1.8 × 10⁻⁸ |
| ZZA4013 | 41 | 4.2 × 10⁻⁴ | 3.2 × 10⁻⁹ | 6.6 × 10⁻⁴ | 3.2 × 10⁻⁸ |
| ZAZ4015 | 42 | 4.0 × 10⁻⁴ | 2.0 × 10⁻⁸ | 1.3 × 10⁻³ | 3.5 × 10⁻⁸ |
| ZZA4053 | 43 | 1.3 × 10⁻² | 9.5 × 10⁻⁸ | 6.2 × 10⁻⁴ | 9.7 × 10⁻⁸ |
| ZZA4054 | 44 | 1.3 × 10⁻² | 1.5 × 10⁻⁷ | 6.3 × 10⁻⁴ | 9.3 × 10⁻⁸ |
| ZAZ4057 | 45 | 9.6 × 10⁻³ | 2.1 × 10⁻⁸ | 6.5 × 10⁻⁴ | 3.1 × 10⁻⁸ |
| ZZA4261 | 46 | n.a. | n.a. | 1.4 × 10⁻³ | 1.1 × 10⁻⁸ |
| ZZA4262 | 47 | n.a. | n.a. | 9.8 × 10⁻⁴ | 5.4 × 10⁻⁹ |
| ZZA4263 | 48 | n.a. | n.a. | 9.5 × 10⁻⁴ | 7.6 × 10⁻⁹ |

¹Relevant K_{d} (the most represented in %)
n.a. Not analyzed

In vitro cell migration assay with dimeric polypeptides: Homo- and heterodimeric polypeptides were analyzed for their ability to inhibit HMGB1 induced cell migration. IC50 values were calculated from experimental data and are shown in Table 10. With this experimental set up, the lowest detection limit for IC50 values is 0.1 nM. The negative homodimeric control polypeptide ZZA4014 (SEQ ID NO:49) had no effect on HMGB1 induced migration.

TABLE 10
Summary of IC50 values polypeptides
inhibiting HMGB1 induced cell migration

| Designation | SEQ ID NO | IC50 (nM) |
|---|---|---|
| ZA3812 | 35 | ≤0.1 |
| ZZA4010 | 38 | 0.1-0.3 |
| ZZA4011 | 39 | ≤0.1 |
| ZZA4012 | 40 | ≤0.1 |
| ZZA4013 | 41 | 0.2 |
| ZAZ4015 | 42 | >1 |
| ZZA4053 | 43 | 0.3-1 |
| ZZA4054 | 44 | >1 |
| ZAZ4057 | 45 | >1 |

### Example 6

### Alanine Scan of HMGB1-Binding Z Variant

### Summary

In this Example, alanine scanning mutagenesis was used to analyze the individual contribution from residues in ZH 01 to the interaction with HMGB1. Each mutated Z variant was analyzed by circular dichroism (CD) spectroscopy by measuring the melting temperature and secondary structure content to ensure preserved structure and stability properties.

### Materials and Methods

Construction of single-point mutation variants of ZH 01 for alanine scan: Single-point mutations of ZH 01 (SEQ ID NO:1) with codon substitution to alanine at residues 9, 10, 11, 13, 14, 17, 18, 24, 27, 28, 32 or 35 were designed. Residue A25 in ZH 01 was mutated to a serine. Synthesis and cloning of the designed Z variants (SEQ ID NO:14-26; FIG. 1) were ordered from DNA2.0 (Newark, Calif.). The Z gene fragments were subcloned into an expression vector resulting in the encoded sequence MGSSHHHHHHLQ-[ZH ##]-VD.

Cultivation of mutated Z variants with His₆-tag: E. coli T7E2 cells were transformed with plasmids containing the gene fragment of each respective mutated Z variant. The resulting recombinant strains were cultivated in media supplemented with 50 μg/ml kanamycin at 30° C. in 50 ml scale using the EnPresso protocol (BioSilta). In order to induce protein expression, IPTG was added to a final concentration of 0.2 mM at OD₆₀₀≈10. After induction, the cultivations were incubated for 16 h. The cells were harvested by centrifugation.

Purification of mutated Z variants with a His₆-tag: The purification and verification of Z variants were performed as described in Example 2.

Circular dichroism (CD) spectroscopy analysis: The measurements were performed essentially as described in Example 3.

Binding analysis of mutated Z variants: A binding analysis was performed using a Biacore 2000 instrument. All alanine mutated Z variants, together with the original Z variant ZH 01, were analyzed against GST-boxA(red), HMGB1(red), boxA(disulfide) and HMGB1(disulfide). The immobilizations and Biacore analyses were performed essentially as described in Example 3. However, in these analyses, the different targets were directly coupled to separate flow cells of different CM5 chip surfaces and a flow rate of 30 μl/min was used. The ligand immobilization levels on the surfaces were 1410 RU for GST-boxA(red), 1380 RU for HMGB1(red), 540 RU for boxA(disulfide) and 2900 RU for HMGB1(disulfide). The analytes, i.e. the Z variants, were each diluted in HBS-EP buffer (or in HBS-EP+2 mM DTT for use in analysis with reduced targets GST-boxA(red) and HMGB1(red)), within a concentration range of 50 to 1350 nM and injected for 3 min, followed by dissociation in running buffer for 35 min. No regeneration buffer was used in the analyses. Instead, the dissociation time was enough for all binders to dissociate from the chip surfaces. HBS-EP was used as running buffer for targets boxA(disulfide) and HMGB1(disulfide) while HBS-EP+2 mM DTT was used for reduced targets GST-boxA(red) and HMGB1(red). The dissociation constants were calculated as in Example 3.

### Results

Production of mutated Z variants: The mutated Z variants with His₆-tag were successfully cloned and expressed as soluble gene products in E. coli. Each DNA construct was verified by DNA sequencing. SDS-PAGE analysis of each final protein preparation showed that these predominantly contained the desired Z variant. The correct identity and molecular weight of each mutated Z variant were confirmed by HPLC-MS analysis.

CD analysis: The CD spectra and melting temperatures (Tm) for 13 mutated Z variants with His₆-tag was analyzed as described in Example 3. The secondary structure contents and refolding properties showed to be unaffected by the introduction of a point mutation. However, the thermal stability was affected to various extents, resulting in a shift in melting temperature with +/−10° C. compared to the original Z variant ZH 01. The melting temperatures (Tm) are summarized in Table 11.

TABLE 11
Melting temperatures (Tm) of mutated Z variants

| Designation | SEQ ID NO | Mutation | Tm (° C.) |
|---|---|---|---|
| ZH01 | 1 | — | 49 |
| ZH14 | 14 | D9A | 59 |
| ZH15 | 15 | W10A | 44 |
| ZH16 | 16 | D11A | 50 |
| ZH17 | 17 | F13A | 43 |
| ZH18 | 18 | W14A | 41 |
| ZH19 | 19 | IVA | 42 |
| ZH20 | 20 | W18A | 55 |
| ZH21 | 21 | D24A | 53 |
| ZH22 | 22 | A25S | 46 |
| ZH23 | 23 | Q27A | 47 |
| ZH24 | 24 | R28A | 47 |
| ZH25 | 25 | Y32A | 56 |
| ZH26 | 26 | E35A | 56 |

Binding analysis of mutated Z variants: The interaction of HMGB1 with ZH 01 and the 13 mutated variants thereof was analyzed in a Biacore instrument by injecting various concentrations of the Z variants over surfaces containing immobilized GST-boxA(red), HMGB1(red), boxA(disulfide) and HMGB1(disulfide), respectively. A summary of the dissociation constants from the experiments, which were obtained by using a 1:1 interaction model, is given in Table 12.

TABLE 12
Dissociation constants for binding of Z variants to GST-
boxA(red), HMGB1(red), boxA(disulfide) and HMGB1(disulfide)

| | | | GST- | | boxA(di- | HMGB1(di- |
|---|---|---|---|---|---|---|
| Z | SEQ | | boxA(red) | HMGB1(red) | sulfide) | sulfide) |
| variant | ID NO | Mutation | K_{D} (M) | K_{D} (M) | K_{D} (M) | K_{D} (M) |
| ZH01 | 1 | — | 2.5 × 10⁻⁸ | 1.2 × 10⁻⁸ | 3.8 × 10⁻⁷ | 1.7 × 10⁻⁷ |
| ZH14 | 14 | D9A | 3.6 × 10⁻⁸ | 1.8 × 10⁻⁸ | 7.7 × 10⁻⁷ | 1.5 × 10⁻⁷ |
| ZH15 | 15 | W10A | n.d. | 5.7 × 10⁻⁷ | n.d. | 1.2 × 10⁻⁵ |
| ZH16 | 16 | D11A | 2.1 × 10⁻⁸ | 1.0 × 10⁻⁸ | 6.6 × 10⁻⁷ | 1.3 × 10⁻⁷ |
| ZH17 | 17 | F13A | n.d. | n.d. | n.d. | 6.8 × 10⁻⁶ |
| ZH18 | 18 | W14A | 3.7 × 10⁻⁷ | 2.9 × 10⁻⁷ | n.d. | 2.2 × 10⁻⁶ |
| ZH19 | 19 | I17A | n.d. | n.d. | n.d. | 3.1 × 10⁻⁶ |
| ZH20 | 20 | W18A | 1.3 × 10⁻⁷ | 9.0 × 10⁻⁸ | 5.0 × 10⁻⁵ | 1.5 × 10⁻⁶ |
| ZH21 | 21 | D24A | 1.1 × 10⁻⁷ | 6.4 × 10⁻⁸ | 1.5 × 10⁻⁶ | 6.7 × 10⁻⁷ |
| ZH22 | 22 | A25S | 1.7 × 10⁻⁸ | 9.0 × 10⁻⁹ | 3.3 × 10⁻⁷ | 1.9 × 10⁻⁷ |
| ZH23 | 23 | Q27A | 6.7 × 10⁻⁸ | 4.1 × 10⁻⁸ | 7.9 × 10⁻⁷ | 8.7 × 10⁻⁷ |
| ZH24 | 24 | R28A | n.d. | 6.8 × 10⁻⁷ | n.d. | 7.8 × 10⁻⁶ |
| ZH25 | 25 | Y32A | n.d. | n.d. | n.d. | 7.8 × 10⁻⁶ |
| ZH26 | 26 | E35A | 2.2 × 10⁻⁷ | 1.1 × 10⁻⁷ | n.d. | 2.4 × 10⁻⁶ |

n.d. not determined due to low response.

### Example 7

### Design and Construction of a Maturation Library of HMGB1 A-Box Binding Z Variants

### Summary

In this Example, a new library was designed based on the HMGB1 A-box binding variant ZH 01, as well as on the result from the alanine scan described in Example 6. The maturation library contained approximately 1×10¹⁰ individual clones.

### Materials and Methods

Design of an affinity maturation HMGB1 A-box library: A new library was designed, in which 13 positions of the Z variant molecules were biased towards amino acid residues based on the sequence of ZH 01 (SEQ ID NO:1). Each position was randomized allowing amino acid residues A, D, E, F, H, I, K, L, N, Q, R, S, T, Y, V, W (excluding C, G, M and P in all positions), with the exception of position 24 in the Z molecule in which amino acid G was also allowed. The amino acid residues based on the sequence of the HMGB1 binding Z variant ZH 01 were spiked at a higher proportion to generate an average mutation frequency of approximately four mutations per molecule. The randomization frequency in each position was also normalized with the results from the alanine scan described above, resulting in less mutations in positions important for HMGB1 binding or for thermal stability and more mutations in positions of less importance (Table 13).

TABLE 13
The design of HMGB1 A-box maturation library. The percentages of the amino
acids used in each of the 13 randomized library positions are indicated

| | | Position in BM | | | | | | | | | | | | |
|---|---|---|---|---|---|---|---|---|---|---|---|---|---|---|
| | | 2 | 3 | 4 | 6 | 7 | 10 | 11 | 17 | 18 | 20 | 21 | 25 | 28 |
| | | Position in full length Z variant | | | | | | | | | | | | |
| Codon | | 9 | 10 | 11 | 13 | 14 | 17 | 18 | 24 | 25 | 27 | 28 | 32 | 35 |
| A | Ala | 3.1 | 1.3 | 3.1 | 1.3 | 1.9 | 1.3 | 1.9 | 1.8 | 53.0 | 3.1 | 1.3 | 1.3 | 1.9 |
| C | Cys | 0 | 0 | 0 | 0 | 0 | 0 | 0 | 0 | 0 | 0 | 0 | 0 | 0 |
| D | Asp | 53.0 | 1.3 | 53.0 | 1.3 | 1.9 | 1.3 | 1.9 | 72.0 | 3.1 | 3.1 | 1.3 | 1.3 | 1.9 |
| E | Glu | 3.1 | 1.3 | 3.1 | 1.3 | 1.9 | 1.3 | 1.9 | 1.8 | 3.1 | 3.1 | 1.3 | 1.3 | 72.0 |
| F | Phe | 3.1 | 1.3 | 3.1 | 81.0 | 1.9 | 1.3 | 1.9 | 1.8 | 3.1 | 3.1 | 1.3 | 1.3 | 1.9 |
| G | Gly | 0 | 0 | 0 | 0 | 0 | 0 | 0 | 1.8 | 0 | 0 | 0 | 0 | 0 |
| H | His | 3.1 | 1.3 | 3.1 | 1.3 | 1.9 | 1.3 | 1.9 | 1.8 | 3.1 | 3.1 | 1.3 | 1.3 | 1.9 |
| I | Ile | 3.1 | 1.3 | 3.1 | 1.3 | 1.9 | 81.0 | 1.9 | 1.8 | 3.1 | 3.1 | 1.3 | 1.3 | 1.9 |
| K | Lys | 3.1 | 1.3 | 3.1 | 1.3 | 1.9 | 1.3 | 1.9 | 1.8 | 3.1 | 3.1 | 1.3 | 1.3 | 1.9 |
| L | Leu | 3.1 | 1.3 | 3.1 | 1.3 | 1.9 | 1.3 | 1.9 | 1.8 | 3.1 | 3.1 | 1.3 | 1.3 | 1.9 |
| M | Met | 0 | 0 | 0 | 0 | 0 | 0 | 0 | 0 | 0 | 0 | 0 | 0 | 0 |
| N | Asn | 3.1 | 1.3 | 3.1 | 1.3 | 1.9 | 1.3 | 1.9 | 1.8 | 3.1 | 3.1 | 1.3 | 1.3 | 1.9 |
| P | Pro | 0 | 0 | 0 | 0 | 0 | 0 | 0 | 0 | 0 | 0 | 0 | 0 | 0 |
| Q | Gln | 3.1 | 1.3 | 3.1 | 1.3 | 1.9 | 1.3 | 1.9 | 1.8 | 3.1 | 53.0 | 1.3 | 1.3 | 1.9 |
| R | Arg | 3.1 | 1.3 | 3.1 | 1.3 | 1.9 | 1.3 | 1.9 | 1.8 | 3.1 | 3.1 | 81.0 | 1.3 | 1.9 |
| S | Ser | 3.1 | 1.3 | 3.1 | 1.3 | 1.9 | 1.3 | 1.9 | 1.8 | 3.1 | 3.1 | 1.3 | 1.3 | 1.9 |
| T | Thr | 3.1 | 1.3 | 3.1 | 1.3 | 1.9 | 1.3 | 1.9 | 1.8 | 3.1 | 3.1 | 1.3 | 1.3 | 1.9 |
| V | Val | 3.1 | 1.3 | 3.1 | 1.3 | 1.9 | 1.3 | 1.9 | 1.8 | 3.1 | 3.1 | 1.3 | 1.3 | 1.9 |
| W | Trp | 3.1 | 81.0 | 3.1 | 1.3 | 72.0 | 1.3 | 72.0 | 1.8 | 3.1 | 3.1 | 1.3 | 1.3 | 1.9 |
| Y | Tyr | 3.1 | 1.3 | 3.1 | 1.3 | 1.9 | 1.3 | 1.9 | 1.8 | 3.1 | 3.1 | 1.3 | 81.0 | 1.9 |

Two oligonucleotides, one forward and one reverse complementary, with complementary 3′-ends were generated using TRIM technology. These oligos were ordered from Ella Biotech GmbH (Martinsried, Germany).

The construction of the library was performed essentially as described earlier (e.g. PCT publication WO2017/072280) in a vector denoted pAY03894, but with the exception that after transformation, the cells were pooled and cultivated in 3 l medium TSB-YE medium, supplemented with 10 μg/ml tetracycline and 100 μg/ml ampicillin. Furthermore, the vector pAY03894 differs from the earlier described library vector in three amino acid positions as follows: Y5F, N52S and D53E, thus including these mutations in the expressed protein Z variants displayed on the phage. The library quality and the distribution of amino acids were verified by sequencing as described in WO2009/077175.

Preparation of phage stock: Cells from a glycerol stock containing the phagemid library were inoculated into 10 l cultivation medium (2.5 g/l (NH₄)₂SO₄; 5.0 g/l yeast extract; 30 g/l tryptone; 2 g/l K₂HPO₄; 3 g/l KH₂PO₄; 1.25 g/l Na₃C₆H₅O₇ 2H₂O; 0.1 ml/l Breox FMT30 antifoaming agent, supplemented with 25 μg/ml carbenicillin, 10 μg/ml tetracycline, 5 ml/l of 1.217 M MgSO₄ and 10 ml of a trace element solution [194 mM FeCl₃; 55 mM ZnSO₄; 10.6 mM CuSO₄; 62.5 mM MnSO₄; 47 mM CaCl₂, dissolved in 1.2 M HCl]. pH was controlled to 7 through the automatic addition of 25% NH₄OH, air was supplemented (10 l/min), and the stirrer was set to keep the dissolved oxygen level above 30%. When the cells reached an optical density at 600 nm (OD₆₀₀) of 0.50, the cultivation was infected using a 10× molar excess of M13K07 helper phage. The cells were incubated for 30 min. Then expression was induced by the addition of IPTG to a concentration of 100 μM. 1 h after induction, the cultivation was supplemented with 25 μg/ml kanamycin, and a glucose-limited fed-batch cultivation was started where a 600 g/l glucose solution was fed to the reactor (15 g/h at the start, 40 g/h after 20 h and to the end of the cultivation). The cultivation was harvested 24 h after the addition of helper phages. The cells in the cultivation were removed by centrifugation (15,900×g, 50 min). The phage particles were precipitated from the supernatant twice in PEG/NaCl, filtered and dissolved in PBS and glycerol as described in Example 1. Phage stocks were stored at −80° C. until use in selection.

### Results

Library construction: The new library was designed based on the HMGB1 binding variant ZH 01 (see Examples 1-3) as well as on the result from the alanine scan described in Example 6. The theoretical size of the designed library was 4.7×10⁷Z variants. The actual size of the library, determined by titration after transformation to E. coli. ER2738 cells, was 1.3×10¹⁰ transformants. The library quality was tested by sequencing of 96 transformants and by comparing their actual sequences with the theoretical design. Sequence analysis of individual library members verified a distribution of codons in accordance with the theoretical design.

### Example 8

### Selection and Screening of Affinity Matured HMGB1 A-Box Binding Z Variants

### Summary

In this Example GST-boxA(red), biotinylated HMGB1(red), alternating targets HMGB1(disulfide) and HMGB1(red), and biotinylated boxA(disulfide) were used as targets in phage display selections using an HMGB1 A-box maturation phage library of Z variants. Selected clones were DNA sequenced, produced in E. coli periplasmic fractions and assayed against different target proteins in ELISA and Biacore.

### Materials and Methods

Biotinylation of proteins: GST-boxA(red), HMGB1(red), boxA(disulfide) and a mouse monoclonal anti-HMGB1(C-terminal part) antibody (DPH1.1, HMGBiotech cat. no. HM-904) were biotinylated using No-Weigh EZ-Link Sulfo-NHS-LC-Biotin as described in Example 1.

Phage display selection of HMGB1 A-box binding Z variants: Phage display selection was performed using a phage stock of the newly produced A-box maturation library. Selections against biotinylated GST-boxA(red), biotinylated HMGB1(red), alternating targets HMGB1(disulfide) and HMGB1(red), and biotinylated boxA(disulfide) were performed in four cycles. In track 1, selection was performed in solution and SA beads were used to catch the b-GST-boxA(red):Z-variant complexes. In track 2, selection was performed on solid phase where SA beads were used to catch the HMGB1(red) prior to selection. In track 3, an alternating target strategy was used. Selection was either performed in solution where SA beads with pre-immobilized b-anti-HMGB1 antibody were used to catch the HMGB1(disulfide) or on solid phase where SA beads were used to catch the HMGB1(red) prior to selection. In track 4 with descendants, selection was performed on solid phase where SA beads were used to catch the b-boxA(disulfide) prior to selection.

As selection proceeded, the tracks were further divided according to target concentration and number and/or time of washes. Phage stock preparation, selection procedure and amplification of phage between selection cycles were performed essentially as described in Example 1. The selection buffer consisted of either PBST 0.1% supplemented with 10% FBS and 2 mM DTT (tracks 1, 2 and track 3 (in cycles using HMGB1(red)) or PBST 0.1% supplemented with 10% FBS and 1.5 μM HSA (tracks 3 (in cycles using HMGB1(disulfide)) and 4). No pre-selection was performed. The washing steps were performed either during 1 min, 1 h, 4 h or overnight and with or without addition of a non-biotinylated version of the target used during the selection. An overview of the selection strategy, describing an increased stringency in subsequent cycles, using a lowered target concentration and an increased number of washes, is shown in Table 14.

TABLE 14
Overview of the selection against HMGB1 using the A-box maturation library

| | | Phage | | | | | Conc. of |
|---|---|---|---|---|---|---|---|
| | | stock from | | Target | Number | Duration | free target |
| | Selection | library or | | conc. | of | of last | in last |
| Cycle | track | selection track | Target protein | (nM) | washes | wash (h) | wash (nM) |
| 1 | 1 | A-box matlib | b-GST-boxA(red) | 100 | 2 | — | — |
| 1 | 2 | A-box matlib | b-HMGB1(red) | 100 | 2 | — | — |
| 1 | 3 | A-box matlib | HMGB1(disulfide) | 10 | 2 | — | — |
| 1 | 4 | A-box matlib | b-boxA(disulfide) | 100 | 2 | — | — |
| 2 | 1-1 | 1 | b-GST-boxA(red) | 10 | 5 | 1 | — |
| 2 | 2-1 | 2 | b-HMGB1(red) | 10 | 5 | 1 | — |
| 2 | 3-1 | 3 | b-HMGB1(red) | 1 | 5 | 1 | — |
| 2 | 3-2 | 3 | HMGB1(disulfide) | 1 | 5 | 1 | — |
| 2 | 4-1 | 4 | b-boxA(disulfide) | 10 | 4 | — | — |
| 3 | 1-1-1 | 1-1 | b-GST-boxA(red) | 1 | 6 | 1 | 100 |
| 3 | 2-1-1 | 2-1 | b-HMGB1(red) | 1 | 6 | 1 | 100 |
| 3 | 3-1-1 | 3-1 | HMGB1(disulfide) | 0.2 | 6 | — | — |
| 3 | 3-2-1 | 3-2 | HMGB1(disulfide) | 0.2 | 6 | — | — |
| 3 | 4-1-1 | 4-1 | b-boxA(disulfide) | 1 | 6 | 1 | 100 |
| 4 | 1-1-1-1 | 1-1-1 | b-GST-boxA(red) | 1 | 10 | 4 | 100 |
| 4 | 1-1-1-2 | 1-1-1 | b-GST-boxA(red) | 1 | 10 | ON | 100 |
| 4 | 2-1-1-1 | 2-1-1 | b-HMGB1(red) | 1 | 6 | 4 | 100 |
| 4 | 2-1-1-2 | 2-1-1 | b-HMGB1(red) | 1 | 6 | ON | 100 |
| 4 | 3-1-1-1 | 3-1-1 | b-HMGB1(red) | 1 | 6 | 4 | 100 |
| 4 | 3-1-1-2 | 3-1-1 | b-HMGB1(red) | 1 | 6 | ON | 100 |
| 4 | 3-2-1-1 | 3-2-1 | HMGB1(disulfide) | 0.2 | 6 | 4 | — |
| 4 | 3-2-1-2 | 3-2-1 | HMGB1(disulfide) | 0.2 | 6 | ON | — |
| 4 | 4-1-1-1 | 4-1-1 | b-boxA(disulfide) | 1 | 6 | 4 | 100 |
| 4 | 4-1-1-2 | 4-1-1 | b-boxA(disulfide) | 1 | 6 | ON | 100 |

ON = overnight

Production of Z variants in periplasmic fractions: The Z variants were produced and the periplasmic fraction of each individual variant was prepared as described in Example 1, with the exception that periplasmic fractions were obtained by heating the bacterial suspensions to 70° C. during 15 min. The final supernatant of the periplasmic extract contained the Z variants as fusions to ABD, expressed as AQHDEALE-[ZH ##]-VDYV-[ABD]-YVPG (SEQ ID NO:131).

ELISA screening of Z variants: The binding of Z variants to HMGB1 was analyzed in ELISA assays. Each Z variant was analyzed against the target used in the selection that it was discovered in. The ELISA was performed essentially as described in Example 1. When GST-boxA(red) or HMGB1(red) were used as target, PBSC+0.5 mM DTT was used for dilution of reagents in the two last steps to ensure reduced conditions during the binding event. Target concentrations used in the assay were 2 nM of b-GST-boxA(red), 10 nM of b-HMGB1(red), 100 nM HMGB1(disulfide) and 10 nM b-boxA(disulfide), respectively. As negative control, a periplasmic fraction containing the fusion protein ABD with no Z fusion partner, i.e. AQHDEALEVDYV-[ABD]-YVPG (SEQ ID NO:132), was used. Periplasm samples containing the primary HMGB1 binding Z variant ZH 01 (SEQ ID.NO:1) in fusion with ABD was included on each plate and analyzed as positive control. The absorbance at 450 nm was measured using a multi-well plate reader, EnSpire (Perkin Elmer).

Sequencing: In parallel with the ELISA screening, all clones were sequenced. PCR fragments were amplified from single colonies, sequenced and analyzed as described in Example 1.

Biacore screening of Z variants: The binding of Z variants to boxA(disulfide) was analyzed in an affinity screening using a Biacore 2000 instrument. A polyclonal goat anti-ABD antibody (goat anti-ABD) was immobilized on CM5 chip surfaces basically as described for other proteins in Example 3. For the kinetic screening, analytes were injected in two steps. First, a Z-ABD (where ABD is GA3) periplasmic extract was injected over the surface at 5 μl/min for 10 min. As a second step, 200 nM boxA(disulfide) was injected at 20 μl/min for 5 min, followed by 10 min of dissociation in running buffer HBS-EP. Glycine-HCl pH 2.0 (GE Healthcare, cat. no. BR100355) was used for regeneration of the antibody surfaces between the cycles. The temperature of the assay was 25° C. Before performing the kinetic analyses, the signal from 200 nM boxA(disulfide) injected over a reference surface containing goat anti-ABD but no Z-ABD sample was subtracted from the sensorgram of Z-ABD binding to boxA(disulfide). Rough screening affinities (K_{D}) were calculated from the reference subtracted 200 nM boxA(disulfide) response using a 1:1 binding model of the BiaEvaluation software. A periplasmic extract of ZH 01 was included in the screening analysis for comparison.

### Results

Phage display selection of HMGB1 binding Z variants: Individual clones were obtained after four cycles of phage display selections against GST-boxA(red), biotinylated HMGB1(red), alternating targets HMGB1(disulfide) and HMGB1(red), and biotinylated boxA(disulfide).

ELISA screening of Z variants: Clones obtained after four cycles of selection were produced in 96-well plates and screened against target used in each selection, respectively. Z variants found to give an average response corresponding to 2×the negative control or higher were considered as positive clones and further subjected to DNA-sequencing.

Sequencing: Sequencing was performed for clones showing a positive response in the ELISA screen against target used in each selection. Each Z variant was given a unique identification number ##, and individual Z variants are referred to as ZH ##. Resulting unique amino acid sequences of the 58 amino acid residues long Z variants are listed in FIG. 1 and in the sequence listing as SEQ ID NO:4-13.

Biacore screening of Z variants: Seven new unique Z variants giving the highest response in ELISA-screen, ZH 04 (SEQ ID NO:4), ZH 05 (SEQ ID NO:5), ZH 06 (SEQ ID NO:6), ZH 07 (SEQ ID NO:7), ZH 08 (SEQ ID NO:8), ZH 09 (SEQ ID NO:9) and ZH 10 (SEQ ID NO:10) were submitted to a Biacore affinity screening together with ZH 01 (SEQ ID NO:1) and ZH 29 (SEQ ID NO:29; its binding motif is identical to that of ZH 01, but the sequence comprises scaffold amino acid variations V1A, D2E, Y5F, N52S and D53E). A single concentration of boxA(disulfide) was injected over each Z-ABD captured from periplasmic extracts on a sensor chip surface containing an anti-ABD antibody. The calculated screening affinities are presented in Table 15.

TABLE 15
Calculated approximate K_{D}
values from Biacore affinity screening

| Z variant | SEQ ID NO | K_{D} (M) |
|---|---|---|
| ZH04 | 4 | 5.4 × 10⁻⁸ (Note 1) |
| ZH05 | 5 | 5.4 × 10⁻⁸ |
| ZH06 | 6 | 7.1 × 10⁻⁸ |
| ZH07 | 7 | 4.4 × 10⁻⁸ (Note 2) |
| ZH29 | 29 | 3.6 × 10⁻⁸ (Note 3) |
| ZH08 | 8 | 3.1 × 10⁻⁸ |
| ZH09 | 9 | 9.1 × 10⁻⁸ |
| ZH10 | 10 | 4.7 × 10⁻⁸ |
| ZH01⁴ | 1 | 4.7 × 10⁻⁹ |

1) Average K_{D} from 2 separate measurements
2) Average K_{D} from 8 separate measurements
3) Average K_{D} from 3 separate measurements
4) Differs in scaffold residues in positions 1 (V), 2 (D), 5 (Y), 52 (N) and 53 (D)

### Example 9

### Design and Construction of a Maturation Library of HMGB1 B-Box Binding Z Variants

### Summary

In this Example, a new library was designed based on the HMGB1 B-box binding variants ZH 30 and ZH 31.

### Materials and Methods

Design of an affinity maturation HMGB1 B-box library: The design of the library was based on the amino acid sequences of the HMGB1 B-box binding Z variants ZH 30 (SEQ ID NO:30) and ZH 31 (SEQ ID NO:31), identified and characterized as described in Example 1 and Example 3. In the new library, 13 variable positions in the Z molecule scaffold were either conserved to certain amino acid residues or randomized according to a strategy based on the sequences of the two Z variants. The design for each amino acid residue of the new library, including six variable amino acid positions (11, 18, 24, 25, 32, and 35) and seven constant amino acid positions (9, 10, 13, 14, 17, 27, and 28) in the Z molecule, are displayed in Table 16. The resulting theoretical library size was 3.4×10⁷ variants. In the theoretical design, an even distribution of the different amino acids was applied within each amino acid position.

TABLE 16
The design of HMGB1 B-box maturation library

| Amino acid | | | |
|---|---|---|---|
| position in | | | |
| the Z | | No of | |
| variant | Randomization | amino | Pro- |
| molecule | (amino acid abbreviations) | acids | portion |
| 9 | A | 1 | 1/1 |
| 10 | W | 1 | 1/1 |
| 11 | A, D, E, F, G, H, I, K, L, M, | 18 | 1/18 |
| | N, Q, R, S, T, V, W, Y | | |
| 13 | E | 1 | 1/1 |
| 14 | Q | 1 | 1/1 |
| 17 | W | 1 | 1/1 |
| 18 | A, D, E, F, G, H, I, K, L, M, | 18 | 1/18 |
| | N, Q, R, S, T, V, W, Y | | |
| 24 | A, D, E, F, G, H, I, K, L, M, | 18 | 1/18 |
| | N, Q, R, S, T, V, W, Y | | |
| 25 | A, D, E, F, G, H, I, K, L, M, | 18 | 1/18 |
| | N, Q, R, S, T, V, W, Y | | |
| 27 | F | 1 | 1/1 |
| 28 | Q | 1 | 1/1 |
| 32 | A, D, E, F, G, H, I, K, L, M, | 18 | 1/18 |
| | N, Q, R, S, T, V, W, Y | | |
| 35 | A, D, E, F, G, H, I, K, L, M, | 18 | 1/18 |
| | N, Q, R, S, T, V, W, Y | | |

Construction of an HMGB1 B-box maturation library: A maturation library for HMGB1 B-box binding Z variants is constructed in analogy to what is described above in Example 7, but using the design described in this Example. Two oligonucleotides, one forward and one reverse complementary, with complementary 3′-ends are generated, for example as described in Example 7. The two oligonucleotides are annealed and extended by PCR, using outer primers, to yield one gene fragment covering 147 bp corresponding to partially randomized helix 1 and 2 and flanked by restriction sites XhoI and SacI. The oligonucleotides are for example ordered from Ella Biotech GmbH. Construction of the library is performed essentially as described in Example 7, in a vector that is electroporated into E. coli that is subsequently cultivated. The library quality and distribution of amino acids are verified by sequencing as described in WO2009/077175.

Preparation of phage stock: Cells from a glycerol stock containing the phagemid library are cultivated and phage stock is produced and prepared as described in for example WO2017/072280 or Example 1 above. Phage stocks are stored at −80° C. until used in selection.

### Expected Results

Design of an affinity maturation HMGB1 B-box library: The new library is designed based on the previously selected HMGB1 B-box binding variants ZH 30 and ZH 31. The theoretical size of the designed library is 3.4×10⁷Z variants.

Construction of an HMGB1 B-box maturation library: A library for maturation of HMGB1 B-box binding Z variants is constructed according to the design presented in this Example. The actual size of the library, determined by titration after transformation to E. coli. ER2738 cells, is for example 5×10⁸ transformants. The library quality is tested by sequencing of 96 transformants and by comparing their actual sequences with the theoretical design. Sequence analysis of individual library members verifies a distribution of codons in accordance with the theoretical design.

Preparation of phage stock: A phage stock of the newly constructed affinity maturation library of HMGB1 B-box binding Z variants is produced and stored at −80° C. until use in selection.

### Example 10

### Selection, Screening and Characterization of Affinity Matured HMGB1 B-Box Binding Z Variants

### Summary

This Example describes the selection, screening and general procedure for subcloning and production of B-box binding Z variants, formatted as monomers and homo- or heterodimers, in fusion with ABD, to be used throughout in characterization experiments below.

### Materials and Methods

Biotinylation of proteins: GST-boxB(red), HMGB1(red), and a mouse monoclonal anti-HMGB1(C-terminal part) antibody are biotinylated using No-Weigh EZ-Link Sulfo-NHS-LC-Biotin as described in Example 1.

Phage display selection of HMGB1 B-box binding Z variants: Phage display selection is performed using a phage stock of the newly produced HMGB B-box affinity maturation library. For example, selections against biotinylated GST-boxB(red), biotinylated HMGB1(red), alternating targets HMGB1(disulfide) and HMGB1(red), and HMGB1(disulfide) are performed in four cycles. Selections are for example done as described in Example 8 with the proteins described above. SA beads are used to catch biotinylated proteins and the mouse monoclonal anti-HMGB1 antibody is used to catch non-biotinylated HMGB1 proteins.

As selections proceed, the tracks are further divided according to target concentration and number and/or time of washes. Phage stock preparation, selection procedure and amplification of phage between selection cycles are performed essentially as described in Example 1 or 8. The selection buffer used is either PBST 0.1% supplemented with 10% FBS and 2 mM DTT (in cycles using GST-boxB(red) or HMGB1(red)) or PBST 0.1% supplemented with 10% FBS and 1.5 μM HSA (in cycles using HMGB1(disulfide)). The washing steps are performed as described in Example 8 with or without addition of a non-biotinylated version of the target used during the selection.

Production of Z variants in periplasmic fractions: The Z variants are produced, and the periplasmic fraction of each individual variant is prepared as described in Example 1. The final supernatant of the periplasmic extract contains the respective Z variant as a fusion to ABD (GA3), expressed as AQHDEALE-[ZH ##]-VDYV-[ABD]-YVPG.

Sequencing: In parallel with the ELISA screening, all clones are sequenced. PCR fragments are amplified from single colonies, sequenced and analyzed as described in Example 1.

Screening of Z variants: The binding of Z variants to HMGB1 may for example be analyzed in ELISA assays against relevant proteins as described in Example 1 and/or in Biacore screening as described in Example 8.

Production and analysis of individual HMGB1 B-box binding Z variants: Z variants considered positive in screening assays are cloned as monomers, homodimers and/or heterodimers comprising for example a second B-box binding Z-variant or an A-box binding Z variant described within the scope of this invention, as well as in fusion with ABD. Production is performed as described in Example 2 and the obtained polypeptides are assayed for binding against relevant HMGB1 proteins using for example Biacore and ELISA, and further characterized in different in vitro and in vivo assays as described in Example 3.

### Results

Phage display selections against for example biotinylated GST-boxB(red), biotinylated HMGB1(red), alternating targets HMGB1(disulfide) and HMGB1(red), and HMGB1(disulfide) are expected to generate new B-box binding Z variants with improved binding properties. Z variants with higher affinity for the B-box of HMGB1 are expected to show enhanced HMGB1 inhibiting potency in in vitro and in vivo models, alone or for example in combination with an A-box binding Z-variant.

### Example 11

### Binding Specificity Study

### Summary

The high-mobility group box (HMGB) family includes four members: HMGB1, HMGB2, HMGB3 and HMGB4. HMGB1 shows 84%, 81% and 42% identity at the amino acid sequence level to HMGB2, HMGB3 and HMGB4, respectively. HMGB1 also shows 86% identity to the SP100 nuclear antigen. In this example, SPR was used to analyze the target binding affinity of ZZA4263 for HMGB1(red), HMGB1(disulfide), HMGB2, HMGB3 and SP100 as well as for tumor necrosis factor (TNF), interleukin-6 (IL-6) and interleukin-1β (IL-1β), the latter three being involved in the HMGB1 signaling pathway.

### Materials and Methods

HSA (Albucult®, Novozyme) was diluted to 10 μg/ml in acetate, pH 4.0, and immobilized onto a CM5 sensor chip using a Biacore 2000 instrument. Immobilization was performed by amine chemistry, using amine coupling kit, according to the manufacturer's instructions. The first surface of each chip was activated and deactivated for use as a reference cell (blank surface) during analyte injections. HBS-EP was used as running buffer, supplemented with 1.5 mM DTT when analyzing the binding to HMGB1(red). ZZA4263 was injected at a concentration of 100 nM for 4 minutes over the HSA and the reference sensor chip surfaces at a constant flow rate of 30 μl/min. This was followed by 4 minutes' injection of 0, 8.3, 25, 75, 225 and 675 nM of HMGB1(red), HMGB1(disulfide), HMGB2 (Biorbyt, cat. no. orb180287), HMGB3 (Biorbyt, cat. no. orb180288), SP100 (Biorbyt, cat. no. orb169341), TNF (R&D Systems, cat. no. 210-TA), IL-6 (R&D Systems, cat. no. 206-IL) and IL-1β (R&D Systems, cat. no. 201-LB/CF), respectively. The proteins were allowed to dissociate for 20 minutes before the chip surfaces were regenerated by two 10 second pulses using 10 mM HCl. The reference surface and the buffer sample were subtracted from all curves. The buffer sample was subtracted to adjust for the dissociation of ZZA4263 from HSA. All curves were fitted to a kinetic 1:1 Langmuir model in the BIA Evaluation software to estimate the binding properties (kₐ, k_{d} and K_{D}).

### Results

ZZA4263 was injected and captured on a surface with immobilized HSA followed by concentration series of HMGB1(red), HMGB1(disulfide), HMGB2, HMGB3, SP100, TNF, IL-6 and IL-1β. The highly homologous human proteins HMGB2, HMGB3, SP100 all bind to ZZA4263, although with 17-550 times lower affinity compared to HMGB1(red). A summary of the kinetic parameters is presented in Table 17. No significant binding was detected to any of the other tested proteins involved in the HMGB1 signaling pathway (TNF, IL-6 and IL-1β). Thus, ZZA4263 shows high specificity to HMGB1 and homologous proteins.

TABLE 17
Kinetic constants for ZZA4263 binding to
HMGB1 and homologous proteins

| | kₐ | k_{d} | K_{D} |
|---|---|---|---|
| Analyte | (1/Ms) | (1/s) | (M) |
| HMGB1(red) | 1.3 × 10⁶ | 1.5 × 10⁻⁴ | 1.2 × 10⁻¹⁰ |
| HMGB1 (disulfide) | 4.9 × 10⁴ | 5.4 × 10⁻⁴ | 1.1 × 10⁻⁸ |
| HMGB2 | 1.8 × 10⁵ | 3.6 × 10⁻⁴ | 2.0 × 10⁻⁹ |
| HMGB3 | 2.0 × 10⁴ | 1.3 × 10⁻³ | 6.6 × 10⁻⁸ |
| SP100 nuclear antigen | 5.7 × 10³ | 2.2 × 10⁻⁴ | 3.8 × 10⁻⁸ |

### Example 12

### Inhibitory Effect of ZZA4263 in CXCR4 In Vitro Cell Assay

### Summary

Blocking of the HMGB1-CXCL12 complex binding to CXCR4 is thought to be beneficial in tissue injury, where an excessive recruitment of leukocytes is supported by HMGB1 (Schiraldi et al (2012) J Exp Med 209:551-63). This Example describes analysis by the cAMP Hunter eXpress CXCR4 CHO-K1 GPCR assay, which includes cells that overexpress the receptor CXCR4. Forskolin-induced release of cAMP activates an enzymatic reaction that produces a luminescent signal, which is inhibited upon HMGB1-CXCL12 binding to CXCR4. The ability of the HMGB1-binding polypeptide ZZA4263 to reverse the inhibition of the signal, via blocking of HMGB1-CXCL12 binding to CXCR4, was demonstrated.

### Materials and Methods

cAMP Hunter eXpress CXCR4 CHO-K1 GPCR Assay kit with reagents was obtained from DiscoverX (cat no 95-0081E2CP2L). cAMP CHO-K1 CXCR4 eXpress cells (DiscoverX, cat no: 95-0081E2) were thawed and seeded into a white 96-well plate, with clear bottom, in 100 μl AssayComplete Cell Plate 2 Reagent. The cells were cultured overnight at 37° C., 5% CO₂.

On the day of the experiment, dilution of HMGB1 (HMGBiotech, cat no: HM001), CXCL12 (DiscoverX, cat no: 92-1011) and ZZA4263 (SEQ ID NO: 48) was performed in a separate dilution plate. First, a serial dilution of HMGB1 (200 nM-0.3 nM) in Cell Assay Buffer with forskolin and tris(2-carboxyethyl)phosphine hydrochloride (TCEP; Sigma-Aldrich) was performed and incubated with CXCL12 at RT for 15 min. Second, a serial dilution of ZZA4263 (1000 nM-0.0006 nM) in PBS was performed and incubated with HMGB1 (10 nM) and CXCL12 (0.4 nM) in Cell Assay Buffer with forskolin and TCEP at RT for 15 min. Cell medium was removed from cells and 30 μl Cell Assay Buffer was added together with 15 μl HMGB1, CXCL12 and ZZA4263 dilutions. Cells were incubated at 37° C., 5% CO₂ for 30 min. cAMP standard was serially diluted and added to the plate after the incubation. All Cell Assay Buffer was removed from the cells and 45 μl of fresh Cell Assay Buffer was added to each well. Subsequently, 15 μl cAMP Antibody Reagent as well as 60 μl cAMP Working Detection Solution including cAMP Lysis Buffer, Substrate Reagent 1, Substrate Reagent 2 and cAMP Solution D were added to each well. After 1 h of incubation, 60 μl cAMP Solution A was added to each well and the cell plate was incubated for additional 3 h in RT. The luminescence was measured in a 96-well plate reader (Enspire).

### Results

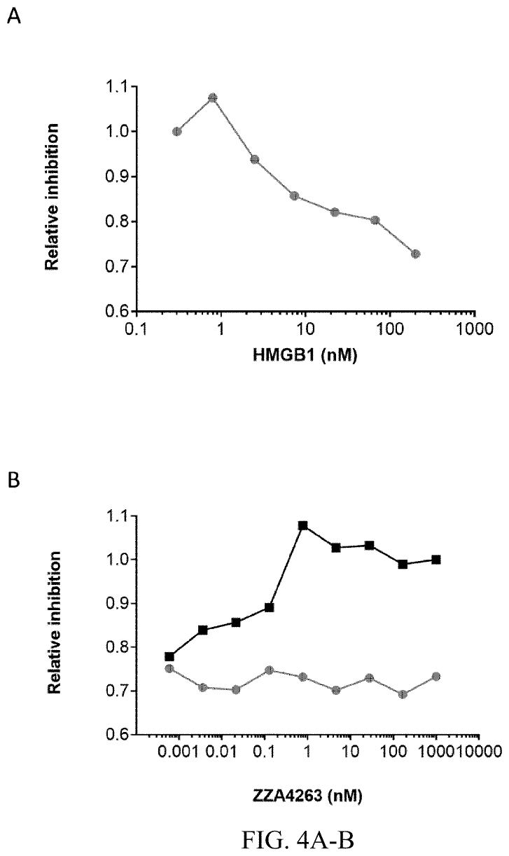

ZZA4263 was investigated for its capacity to block the HMGB1-CXCL12 complex binding to CXCR4, in the cAMP Hunter eXpress CXCR4 CHO-K1 GPCR Assay. Firstly, as demonstrated by FIG. 4A, HMGB1 was shown to bind to CXCR4 in the presence of CXCL12 and to inhibit the luminescent signal in a dose-dependent manner. Secondly, as demonstrated by FIG. 4B, ZZA4263 was shown to inhibit the binding of the HMGB1-CXCL12 complex to CXCR4 in a dose dependent manner, where release of the inhibition enables the luminescent signal to return. The PBS negative control did not show any inhibition.

### Example 13

### Pharmacokinetic Study of ZZA4263 in Mouse and Rat

### Summary

This Example describes the pharmacokinetic (PK) profiles of ZZA4263 obtained in mice and rats after intravenous (i.v.) or intraperitoneal (i.p) administration (mouse only). ZZA4263 was administered at a dose of 1.6 mg/kg in both species. Blood samples were collected up to three weeks post dose and analyzed using an antibody based sandwich PK-ELISA.

### Materials and Methods

PK study in mouse: Six C57BL/6J mice (Charles River) were divided into two groups for i.p. or i.v. administration. ZZA4263 was prepared in PBS and administered as a bolus injection through tail vein or i.p. at 1.6 mg/kg (83 nmol/kg) at a slow and steady rate. The dosing volume administered was 5 and 20 ml/kg for i.v. and i.p. respectively. Serum samples were collected from tail vein of each mouse at time points 0 (predose), 5 min, 8 h, 48 h and 120 h. To prepare sera, the blood samples were left at RT for at least 30 min before centrifugation at 2000×g for 5 min. Sera were extracted and transferred into pre-labelled test tubes and stored at −20° C.

PK study in rat: Six female Sprague Dawley (SD) rats were divided into two groups for crosswise sampling. ZZA4263 was administered as a bolus injection through tail vein at 1.6 mg/kg (83 nmol/kg) at a slow and steady rate. The dosing volume administered was 1 ml/kg. Serum samples (0.2-0.3 ml) were collected from tail vein of each rat at time points 0 (predose), 30 min, 3 h, 24 h, 120 h, 216 h, 336 h and 456 h for group 1 and at time points 0 (predose), 5 min, 1 h, 8 h, 72 h, 168 h, 264 h, 408 h and 504 h for group 2. To prepare sera, the blood samples were left at RT for at least 30 min before centrifugation at 2000×g for 5 min. Sera were extracted and transferred into pre-labelled test tubes and stored at −20° C.

Quantification by ELISA: 96-well half area plates were coated with mouse anti-Z monoclonal antibody (2 μg/ml) in PBS (50 μl/well) and incubated overnight at 4° C. Plates were washed in PBST and blocked with PBSC for 1.5 h. ZZA4263 standard and serum samples were added to the plates (50 μl/well) and incubated for 1.5 h at RT. The ZZA4263 standard was diluted at 10 different concentrations between 1.3-50 μM in PBSC+1% mouse or rat serum pool. Serum samples were diluted 100× (minimal required dilution) in PBSC and titrated in 1:2 steps in PBSC+1% mouse or rat serum pool. Two quality control samples in PBSC+1% mouse or rat serum pool to a concentration of 40 and 4 μM, respectively, were included on each plate. Following washing with PBST, a goat anti-ABD polyclonal antibody (2 μg/ml; 50 μl/well) was added. After incubation for 1 h, the plates were washed and anti-goat IgG-HRP (100 ng/ml; 50 μl/well) added to each well. After one additional hour of incubation and subsequent washing, the plates were developed TMB (50 μl/well) and the reactions were stopped with 2M H₂SO₄ (50 μl/well). The absorbance at 450 nm was measured in a 96-well plate reader (Victor3). Data analysis was performed using Graph Pad Prism and Excel software.

### Results

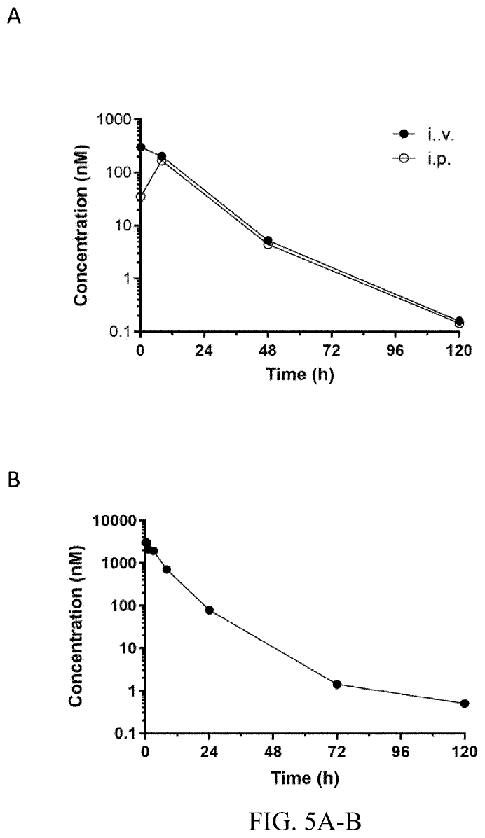

The PK profiles of ZZA4263 in mice after i.v. and i.p. administration, respectively, are shown in FIG. 5A. The bioavailability after i.p. administration was 67% of i.v. administration and the terminal half-life appears to be the same when comparing the two different administration routes. The PK profile of ZZA4263 in rat after i.v. administration is shown in FIG. 5B and the estimated t½ was 25-36 h.

### Example 14

### In Vivo Evaluation of ZZA4263 in a CLP-Induced Sepsis Model

### Summary

The Cecal Ligation and Puncture (CLP) model is a standard animal model of microbial sepsis. The study described in this Example primarily assessed the capacity of the HMGB-1 binding polypeptide ZZA4263 to reduce mortality in CLP-induced sepsis in mice.

### Materials and Methods

The animal study in C57BL mice was performed at Pharmaseed Ltd. (Israel) in compliance with The Israel Animal Welfare Act and following The Israel Board for Animal Experiments Ethics Committee approval # IL-19-1-49. On day 1, sepsis was induced following a modification of a previously published method of CLP (Ruiz et al (2016), Intensive Care Medicine Experimental 4:22-35). Sham operated control mice (n=10) were anesthetized and underwent laparotomy without puncture or cecal ligation. Sepsis induced mice were divided into groups of twenty. CLP is a harsh procedure and animals that either died or showed severe symptoms with low chance of recovery were excluded on humane grounds before treatment initiation (or before second dose). 0.14 mg/kg ZZA4263, 1.4 mg/kg ZZA4263, 25 mg/kg imipenem/cilastatin (antibiotics positive control) and vehicle (PBS), respectively, were administered i.p. to mice on day 2, 3, 4 and 5 (first dose 24 h after the surgery). Sham-operated mice did not receive any treatment. Until termination of the study on day 7, mice were monitored with regard to morbidity and mortality (twice daily), Murine Sepsis Score (MSS; twice daily), body weight (once daily) and body temperature (twice daily).

### Results

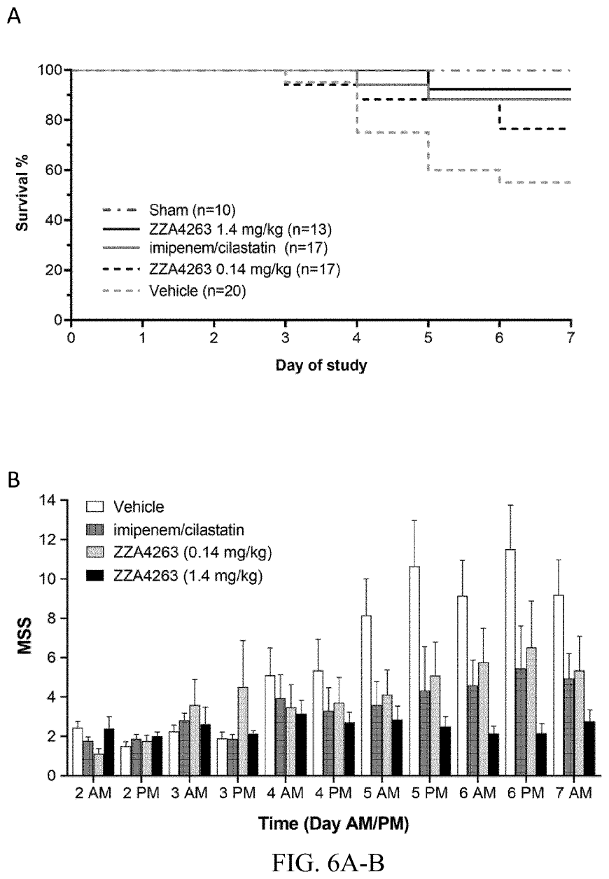

The effect of the HMGB1 binding polypeptide ZZA4263 in the treatment of sepsis was assessed in the CLP model in mice. ZZA4263 demonstrated dose dependent increased survival rate (FIG. 6A) and lower MSS, i.e. reduced severity of sepsis (FIG. 6B), compared to vehicle. Both dose groups that received ZZA4263 showed a lower MSS compared to the group receiving positive control imipenem/cilastatin. The higher dose group, i.e. 1.4 mg/kg of ZZA4263, also showed an increased survival rate compared to imipenem/cilastatin. All mice subjected to CLP suffered from pronounced weight loss during the whole study period, but no significant differences were observed between the treatment groups. Sham operated mice lost weight after the surgery, but their body weight recovered to baseline by the end of the study. Body temperature was comparable in all groups. The temperature remained stable during the study except for animals that were in near-death condition for which the body temperature dropped drastically.

### Itemized List of Embodiments

1. HMGB1 binding polypeptide, comprising an HMGB1 binding motif BM, which motif consists of an amino acid sequence selected from:

i)

(SEQ ID NO: 56)
EX₂X₃X₄AX₆X₇EIX₁₀X₁₁LPNLX₁₆
X₁₇X₁₈QX₂₀X₂₁AFIYX₂₆LED

wherein, independently of each other,
X₂ is selected from A, D, S and T;
X₃ is selected from E, R and W;
X₄ is selected from A, D and Q;
X₆ is selected from F and M;
X₇ is selected from E, H, W and Y;
X₁₀ is selected from I and L;
X₁₁ is selected from A and W;
X₁₆ is selected from N and T;
X₁₇ is selected from A, D, N and W;
X₁₈ is selected from A, E, Q, R, S, T and Y;
X₂₀ is selected from A and Q;
X₂₁ is selected from K, L and R; and
X₂₆ is selected from K and S;
   and
   ii) an amino acid sequence which has at least 93% identity to the sequence defined in i).

2. HMGB1 binding polypeptide according to item 1, wherein in sequence i)
X₂ is selected from A, D, S and T;
X₃ is selected from E, R and W;
X₄ is selected from D and Q;
X₆ is selected from F and M;
X₇ is selected from E, H, W and Y;
X₁₀ is selected from I and L;
X₁₁ is W;
X₁₆ is selected from N and T;
X₁₇ is selected from D, N and W;
X₁₈ is selected from A, E, Q, R, T and Y;
X₂₀ is Q;
X₂₁ is selected from K, L and R; and
X₂₆ is selected from K and S.

3. HMGB1 binding polypeptide according to any preceding item, wherein in sequence i)
X₂ is selected from A, D, S and T;
X₃ is selected from E, R and W;
X₄ is selected from D and Q;
X₆ is selected from F and M;
X₇ is selected from E, H, W and Y;
X₁₀ is selected from I and L;
X₁₁ is W;
X₁₆ is selected from N and T;
X₁₇ is selected from D, N and W;
X₁₈ is selected from A, E, Q, R and T;
X₂₀ is Q;
X₂₁ is selected from K, L and R; and
X₂₆ is selected from K and S.
   4. HMGB1 binding polypeptide according to any preceding item, wherein in sequence i)
X₂ is selected from A and D;
X₃ is selected from E and W;
X₄ is selected from D and Q;
X₆ is selected from F and M;
X₇ is selected from E, H and W;
X₁₀ is selected from I and L;
X₁₁ is W;
X₁₆ is selected from N and T;
X₁₇ is selected from D, N and W;
X₁₈ is selected from A, E, and T;
X₂₀ is Q;
X₂₁ is selected from K, L and R; and
X₂₆ is selected from K and S.

5. HMGB1 binding polypeptide according to any one of items 1-3, wherein in sequence i)
X₂ is selected from A, D, S and T;
X₃ is selected from R and W;
X₄ is D;
X₆ is F;
X₇ is selected from E, Y and W;
X₁₀ is I;
X₁₁ is W;
X₁₆ is selected from N and T;
X₁₇ is D;
X₁₈ is selected from A, Q and R;
X₂₀ is Q;
X₂₁ is R; and
X₂₆ is selected from K and S.

6. HMGB1 binding polypeptide according to any preceding item, wherein sequence i) corresponds to the sequence from position 8 to position 36 in a sequence selected from the group consisting of SEQ ID NO:1-14, 16 and 20-23.

7. HMGB1 binding polypeptide according to item 6, wherein sequence i) corresponds to the sequence from position 8 to position 36 in a sequence selected from the group consisting of SEQ ID NO:1-14.

8. HMGB1 binding polypeptide according to item 7, wherein sequence i) corresponds to the sequence from position 8 to position 36 in a sequence selected from the group consisting of SEQ ID NO:1-10.

9. HMGB1 binding polypeptide according to item 8, wherein sequence i) corresponds to the sequence from position 8 to position 36 in a sequence selected from the group consisting of SEQ ID NO:1-3.

10. HMGB1 binding polypeptide according to any one of items 6-7, wherein sequence i) corresponds to the sequence from position 8 to position 36 in a sequence selected from the group consisting of SEQ ID NO:4-13.

11. HMGB1 binding polypeptide according to any one of items 7-9, wherein sequence i) corresponds to the sequence from position 8 to position 36 in SEQ ID NO:1.

12. HMGB1 binding polypeptide according to any preceding item, which binds to the A-box of HMGB1 such that the K_{D} value of the interaction is at most 1×10⁻⁶ M, for example at most 5×10⁻⁷ M, for example at most 1×10⁻⁷ M, for example at most 5×10⁻⁸ M, for example at most 1×10⁻⁸ M.

13. HMGB1 binding polypeptide according to any preceding item, which binds to the A-box of HMGB1 such that the half maximal effective concentration of the interaction, in a suitable assay of the effect, is at most 1×10⁻⁶ M, for example at most 5×10⁻⁷ M, for example at most 1×10⁻⁷ M, for example at most 5×10⁻⁸ M, for example at most 1×10⁻⁸ M.

14. HMGB1 binding polypeptide according to any preceding item, which binds to the HMGB1 A-box region in its reduced state.

15. HMGB1 binding polypeptide according to any preceding item, which binds to the HMGB1 A-box region in its oxidized state.

16. HMGB1 binding polypeptide, comprising an HMGB1 binding motif BM, which motif consist of an amino acid sequence selected from:

iii)

(SEQ ID NO: 57)
EAWX₄AEQEIW X₁₁LPNLX₁₆X₁₇X₁₈
QF QAFIX₂₅X₂₆LX₂₈D

wherein each of X₄, X₁₁, X₁₇, X₁₈, X₂₅, and X₂₈ are, independently of each other, selected from A, D, E, F, G, H, I, K, L, M, N, Q, R, S, T, V,
W and Y;
X₁₆ is selected from N and T; and
X₂₆ is selected from K and S;
   and
   iv) an amino acid sequence which has at least 93% identity to the sequence defined in iii).

17. HMGB1 binding polypeptide according to item 16, wherein in sequence iii)
X₄ is selected from L and S;
X₁₁ is selected from D and Q;
X₁₆ is selected from N and T;
X₁₇ is selected from E and L;
X₁₈ is selected from A and Q;
X₂₅ is selected from L and M;
X₂₆ is selected from K and S; and
X₂₈ is selected from I and L.

18. HMGB1 binding polypeptide according to any one of items 16-17, wherein sequence iii) corresponds to the sequence from position 8 to position 36 in a sequence selected from the group consisting of SEQ ID NO:30 and SEQ ID NO:31.

19. HMGB1 binding polypeptide according to item 18, wherein sequence iii) corresponds to the sequence from position 8 to position 36 in SEQ ID NO:31.

20. HMGB1 binding polypeptide according to any one of items 16-19, which binds t to the B-box of HMGB1 such that the K_{D} value of the interaction is at most 1×10⁻⁶ M, for example at most 5×10⁻⁷ M, for example at most 1×10⁻⁷ M, for example at most 5×10⁻⁸ M, for example at most 1×10⁻⁸ M.

21. HMGB1 binding polypeptide according to item 20, which binds to the B-box of HMGB1 such that the half maximal effective concentration of the interaction, in a suitable assay of the effect, is at most 1×10⁻⁶ M, for example at most 5×10⁻⁷ M, for example at most 1×10⁻⁷ M, for example at most 5×10⁻⁸ M, for example at most 1×10⁻⁸ M.

22. HMGB1 binding polypeptide according to any preceding item, wherein said HMGB1 binding motif forms part of a three-helix bundle protein domain.

23. HMGB1 binding polypeptide according to item 22, wherein said HMGB1 binding motif essentially forms part of two helices with an interconnecting loop, within said three-helix bundle protein domain.

24. HMGB1 binding polypeptide according to item 23, wherein said three-helix bundle protein domain is selected from bacterial receptor domains.

25. HMGB1 binding polypeptide according to item 24, wherein said three-helix bundle protein domain is selected from domains of protein A from Staphylococcus aureus or derivatives thereof.

26. HMGB1 binding polypeptide according to any preceding item, which comprises a binding module BMod, the amino acid sequence of which is selected from:

v)

(SEQ ID NO: 58)
K-[BM]-DPSQSXₐX_{b}LLX_{c} EAKKLX_{d}XₑX_{f}Q;

wherein
[BM] is an HMGB1 binding motif as defined in any one of items 1-11 and 16-19;
Xₐ is selected from A and S;
X_{b} is selected from N and E;
X_{c} is selected from A, S and C;
X_{d} is selected from E, N and S;
Xₑ is selected from D, E and S; and
X_{f} is selected from A and S; and
   vi) an amino acid sequence which has at least 93% identity to a sequence defined in v).

27. HMGB1 binding polypeptide according to item 26, wherein sequence v) corresponds to the sequence from position 7 to position 55 in a sequence selected from the group consisting of SEQ ID NO:1-14, 16, 20-23 and 27-29.

28. HMGB1 binding polypeptide according to item 27, wherein sequence v) corresponds to the sequence from position 7 to position 55 in a sequence selected from the group consisting of SEQ ID NO:1-14 and 29.

29. HMGB1 binding polypeptide according to item 28, wherein sequence v) corresponds to the sequence from position 7 to position 55 in a sequence selected from the group consisting of SEQ ID NO:1-14.

30. HMGB1 binding polypeptide according to item 29, wherein sequence v) corresponds to the sequence from position 7 to position 55 in a sequence selected from the group consisting of SEQ ID NO:1-10.

31. HMGB1 binding polypeptide according to item 30, wherein sequence v) corresponds to the sequence from position 7 to position 55 in a sequence selected from the group consisting of SEQ ID NO:1-3.

32. HMGB1 binding polypeptide according to any one of items 27-29, wherein sequence v) corresponds to the sequence from position 7 to position 55 in a sequence selected from the group consisting of SEQ ID NO:4-13.

33. HMGB1 binding polypeptide according to any one of items 27-31, wherein sequence v) corresponds to the sequence from position 7 to position 55 in SEQ ID NO:1.

34. HMGB1 binding polypeptide according to any one of items 27-28, wherein sequence v) corresponds to the sequence from position 7 to position 55 in SEQ ID NO:29.

35. HMGB1 binding polypeptide according to item 26, wherein sequence v) corresponds to the sequence from position 7 to position 55 in a sequence selected from the group consisting of SEQ ID NO:30-32.

36. HMGB1 binding polypeptide according to item 35, wherein sequence v) corresponds to the sequence from position 7 to position 55 in SEQ ID NO:31.

37. HMGB1 binding polypeptide according to any preceding item, which comprises an amino acid sequence selected from:

vii) YA-[BMod]-AP;

wherein [BMod] is an HMGB1 binding module as defined in any one of items 26-36; and

viii) an amino acid sequence which has at least 90% identity to a sequence defined in vii).

38. HMGB1 binding polypeptide according to item 37, which comprises an amino acid sequence selected from:

ix)

(SEQ ID NO: 59)
VDAKYAK-[BM]-DPSQSSELLSEAKKLNDSQAPK;

wherein [BM] is an HMGB1 binding motif as defined in any one of items 1-11 and 16-19; and

x) an amino acid sequence which has at least 89% identity to the sequence defined in ix).

39. HMGB1 binding polypeptide according to item 38, wherein sequence ix) is selected from the group consisting of SEQ ID NO:1-3 and 14-26.

40. HMGB1 binding polypeptide according to item 39, wherein sequence ix) is selected from the group consisting of SEQ ID NO:1-3.

41. HMGB1 binding polypeptide according to item 40, wherein sequence ix) is SEQ ID NO:1.

42. HMGB1 binding polypeptide according to item 38, wherein sequence ix) is selected from the group consisting of SEQ ID NO:30-31.

43. HMGB1 binding polypeptide according to item 42, wherein sequence ix) is SEQ ID NO:31.

44. HMGB1 binding polypeptide according to item 37, which comprises an amino acid sequence selected from:

xi)

(SEQ ID NO: 60)
AEAKYAK-[BM]-DPSQSSELLSEAKKLNDSQAPK,

wherein [BM] is an HMGB1 binding motif as defined in any one of items 1-11 and 16-19; and

xii) an amino acid sequence which has at least 89% identity to the sequence defined in xi).

45. HMGB1 binding polypeptide according to item 44, wherein sequence xi) is selected from the group consisting of SEQ ID NO:27-28.

46. HMGB1 binding polypeptide according to item 45, wherein sequence xi) is SEQ ID NO:27.

47. HMGB1 binding polypeptide according to item 44, wherein sequence xi) is SEQ ID NO:32.

48. HMGB1 binding polypeptide according to any one of items 1-36, which comprises an amino acid sequence selected from:

xiii) FA-[BMod]-AP;

wherein [BMod] is an HMGB1 binding module as defined in any one of items 26-36; and

xiv) an amino acid sequence which has at least 90% identity to a sequence defined in xiii).

49. HMGB1 binding polypeptide according to item 48, which comprises an amino acid sequence selected from:

xv)

(SEQ ID NO: 61)
AEAKFAK-[BM]-DPSQSSELLSEAKKLSESQAPK;

wherein [BM] is an HMGB1 binding motif as defined in any one of items 1-11 and 16-19; and

xvi) an amino acid sequence which has at least 89% identity to the sequence defined in xv).

50. HMGB1 binding polypeptide according to item 49, wherein sequence xv) is selected from the group consisting of SEQ ID NO:4-13 and 29.

51. HMGB1 binding polypeptide according to item 48, which comprises an amino acid sequence selected from:

xvii)

(SEQ ID NO: 62)
AEAKFAK-[BM]-DPSQSSELLSEAKKLNDSQAPK;

wherein [BM] is an HMGB1 binding motif as defined in any one of items 1-11 and 16-19; and

xviii) an amino acid sequence which has at least 89% identity to the sequence defined in xvii).

52. HMGB1 binding polypeptide according to any preceding item, which comprises an amino acid sequence selected from:

(SEQ ID NO: 63)
ADNNFNK-[BM]-DPSQSANLLSEAKKLNESQAPK;
(SEQ ID NO: 64)
ADNKFNK-[BM]-DPSQSANLLAEAKKLNDAQAPK;
(SEQ ID NO: 65)
ADNKFNK-[BM]-DPSVSKEILAEAKKLNDAQAPK;
(SEQ ID NO: 66)
ADAQQNNFNK-[BM]-DPSQSTNVLGEAKKLNESQAPK;
(SEQ ID NO: 67)
AQHDE-[BM]-DPSQSANVLGEAQKLNDSQAPK;
(SEQ ID NO: 68)
VDNKFNK-[BM]-DPSQSANLLAEAKKLNDAQAPK;
(SEQ ID NO: 69)
AEAKYAK-[BM]-DPSESSELLSEAKKLNKSQAPK;
(SEQ ID NO: 70)
VDAKYAK-[BM]-DPSQSSELLAEAKKLNDAQAPK;
(SEQ ID NO: 71)
VDAKYAK-[BM]-DPSQSSELLAEAKKLNDSQAPK;
(SEQ ID NO: 72)
AEAKYAK-[BM]-DPSQSSELLSEAKKLNDSQAPK;
(SEQ ID NO: 73)
AEAKYAK-[BM]-DPSQSSELLSEAKKLNDSQAP;
(SEQ ID NO: 74)
AEAKFAK-[BM]-DPSQSSELLSEAKKLNDSQAPK;
(SEQ ID NO: 75)
AEAKFAK-[BM]-DPSQSSELLSEAKKLNDSQAP;
(SEQ ID NO: 76)
AEAKYAK-[BM]-DPSQSSELLAEAKKLNDAQAPK;
(SEQ ID NO: 77)
AEAKYAK-[BM]-DPSQSSELLSEAKKLSESQAPK;
(SEQ ID NO: 78)
AEAKYAK-[BM]-DPSQSSELLSEAKKLSESQAP;
(SEQ ID NO: 79)
AEAKFAK-[BM]-DPSQSSELLSEAKKLSESQAPK;
(SEQ ID NO: 80)
AEAKFAK-[BM]-DPSQSSELLSEAKKLSESQAP;
(SEQ ID NO: 81)
AEAKYAK-[BM]-DPSQSSELLAEAKKLSEAQAPK;
(SEQ ID NO: 82)
AEAKYAK-[BM]-QPEQSSELLSEAKKLSESQAPK;
(SEQ ID NO: 83)
AEAKYAK-[BM]-DPSQSSELLSEAKKLESSQAPK;
(SEQ ID NO: 84)
AEAKYAK-[BM]-DPSQSSELLSEAKKLESSQAP;
(SEQ ID NO: 85)
AEAKYAK-[BM]-DPSQSSELLAEAKKLESAQAPK;
(SEQ ID NO: 86)
AEAKYAK-[BM]-QPEQSSELLSEAKKLESSQAPK;
(SEQ ID NO: 87)
AEAKYAK-[BM]-DPSQSSELLSEAKKLSDSQAPK;
(SEQ ID NO: 88)
AEAKYAK-[BM]-DPSQSSELLSEAKKLSDSQAP;
(SEQ ID NO: 89)
AEAKYAK-[BM]-DPSQSSELLAEAKKLSDSQAPK;
(SEQ ID NO: 90)
AEAKYAK-[BM]-DPSQSSELLAEAKKLSDAQAPK;
(SEQ ID NO: 91)
AEAKYAK-[BM]-QPEQSSELLSEAKKLSDSQAPK;
(SEQ ID NO: 92)
VDAKYAK-[BM]-DPSQSSELLSEAKKLNDSQAPK;
(SEQ ID NO: 93)
VDAKYAK-[BM]-DPSQSSELLAEAKKLNDAQAPK;
(SEQ ID NO: 94)
VDAKYAK-[BM]-DPSQSSELLSEAKKLSESQAPK;
(SEQ ID NO: 95)
VDAKYAK-[BM]-DPSQSSELLAEAKKLSEAQAPK;
(SEQ ID NO: 96)
VDAKYAK-[BM]-QPEQSSELLSEAKKLSESQAPK;
(SEQ ID NO: 97)
VDAKYAK-[BM]-DPSQSSELLSEAKKLESSQAPK;
(SEQ ID NO: 98)
VDAKYAK-[BM]-DPSQSSELLAEAKKLESAQAPK;
(SEQ ID NO: 99)
VDAKYAK-[BM]-QPEQSSELLSEAKKLESSQAPK;
(SEQ ID NO: 100)
VDAKYAK-[BM]-DPSQSSELLSEAKKLSDSQAPK;
(SEQ ID NO: 101)
VDAKYAK-[BM]-DPSQSSELLAEAKKLSDSQAPK;
(SEQ ID NO: 102)
VDAKYAK-[BM]-DPSQSSELLAEAKKLSDAQAPK;
(SEQ ID NO: 103)
VDAKYAK-[BM]-QPEQSSELLSEAKKLSDSQAPK;
(SEQ ID NO: 104)
VDAKYAK-[BM]-DPSQSSELLAEAKKLNKAQAPK;
(SEQ ID NO: 105)
AEAKYAK-[BM]-DPSQSSELLAEAKKLNKAQAPK;
and
(SEQ ID NO: 106)
ADAKYAK-[BM]-DPSQSSELLSEAKKLNDSQAPK;

wherein [BM] is an HMGB1 binding motif as defined in any one of items 1-11 and 16-19.

53. HMGB1 binding polypeptide according to any preceding item, wherein said HMGB1 is human HMGB1.

54. HMGB1 binding polypeptide according to any preceding item which comprises additional amino acids at the C-terminal and/or N-terminal end.

55. HMGB1 binding polypeptide according to item 54, wherein said additional amino acid(s) improve(s) production, purification, stabilization in vivo or in vitro, coupling or detection of the polypeptide.

56. HMGB1 binding polypeptide according to any preceding item in multimeric form, comprising at least two HMGB1 binding polypeptide monomer units, whose amino acid sequences may be the same or different.

57. HMGB1 binding polypeptide according to item 56, wherein a first HMGB1 binding polypeptide monomer unit is an HMGB1 binding polypeptide according to any one of items 1-15, and wherein a second HMGB1 binding polypeptide monomer unit is an HMGB1 binding polypeptide according to any one of items 16-21.

58. HMGB1 binding polypeptide according to any one of items 56-57, wherein a first HMGB1 binding polypeptide monomer unit has affinity for the A-box of HMGB1, and wherein a second HMGB1 binding polypeptide monomer unit has affinity for the B-box of HMGB1.

59. HMGB1 binding polypeptide according to any one of items 56-58, wherein said HMGB1 binding polypeptide monomer units are covalently coupled together.

60. HMGB1 binding polypeptide according to item 59, wherein the HMGB1 binding polypeptide monomer units are expressed as a fusion protein.

61. HMGB1 binding polypeptide according to any one of items 56-60, in dimeric form.

62. Fusion protein or conjugate comprising
a first moiety consisting of an HMGB1 binding polypeptide according to any preceding item; and
a second moiety consisting of a polypeptide having a desired biological activity.

63. Fusion protein or conjugate according to item 62, wherein said desired biological activity is a therapeutic activity.

64. Fusion protein or conjugate according to item 62, wherein said desired biological activity is a binding activity.

65. Fusion protein or conjugate according to item 62, wherein said desired biological activity is an enzymatic activity.

66. Fusion protein or conjugate according to item 64, wherein said binding activity is albumin binding activity which increases in vivo half-life of the fusion protein or conjugate.

67. Fusion protein or conjugate according to item 66, wherein said second moiety comprises the albumin binding domain of streptococcal protein G or a derivative thereof.

68. Fusion protein or conjugate according to item 64, wherein said binding activity acts to block a biological activity.

69. Fusion protein or conjugate according to item 63, wherein the second moiety is a therapeutically active polypeptide.

70. Fusion protein or conjugate according to any one of items 62-65 and 68-69, wherein the second moiety is selected from the group consisting of human endogenous enzymes, hormones, growth factors, chemokines, cytokines and lymphokines.

71. Fusion protein or conjugate according to any one of items 62-65 and 68-69, wherein the second moiety is selected from the group consisting of an antibody and an antigen binding fragment thereof.

72. Fusion protein or conjugate according to item 71, wherein said at least one antibody or antigen binding fragment thereof is selected from the group consisting of full-length antibodies, Fab fragments, Fab′ fragments, F(ab′)₂ fragments, Fc fragments, Fv fragments, single chain Fv (scFv) fragments, (scFv)₂ and domain antibodies.

73. Fusion protein or conjugate according to item 72, wherein said at least one antibody or antigen binding fragment thereof is selected from the group consisting of full-length antibodies, Fab fragments and scFv fragments.

74. Fusion protein or conjugate according to item 73, wherein said at least one antibody or antigen binding fragment thereof is a full-length antibody.

75. Fusion protein according to any one of items 62-74, wherein the second moiety further comprises a linker.

76. HMGB1 binding polypeptide, fusion protein or conjugate according to any preceding item, further comprising a label.

77. HMGB1 binding polypeptide, fusion protein or conjugate according to item 76, wherein said label is selected from the group consisting of fluorescent dyes and metals, chromophoric dyes, chemiluminescent compounds and bioluminescent proteins, enzymes, radionuclides, radioactive particles and pretargeting recognition tags.

78. HMGB1 binding polypeptide, fusion protein or conjugate according to item 77, comprising a chelating environment provided by a polyaminopolycarboxylate chelator conjugated to the HMGB1 binding polypeptide via a thiol group of a cysteine residue or an amine group of a lysine residue.

79. HMGB1 binding polypeptide, fusion protein or conjugate according to item 78, which comprises a pretargeting recognition tag forming part of a complementary pair of pretargeting moieties, for example selected from stept(avidin)/biotin, oligonucleotide/complementary oligonucleotide such as DNA/complementary DNA, RNA/complementary RNA, phosphorothioate nucleic acid/complementary phosphorothioate nucleic acid and peptide nucleic acid/complementary peptide nucleic acid and morpholinos/complementary morpholinos.

80. A polynucleotide encoding a polypeptide according to any one of items 1-75.

81. Expression vector comprising a polynucleotide according to item 80.

82. Host cell comprising an expression vector according to item 81.

83. Method of producing a polypeptide according to any one of items 1-75, comprising
culturing a host cell according to item 82 under conditions permissive of expression of said polypeptide from said expression vector, and
isolating said polypeptide.

84. Composition comprising an HMGB1 binding polypeptide, fusion protein or conjugate according to any one of items 1-79 and at least one pharmaceutically acceptable excipient or carrier.

85. HMGB1 binding polypeptide, fusion protein or conjugate according to any one of items 1-79 or a composition according item 84 for oral, topical, intravenous, intraperitoneal, subcutaneous, pulmonary, transdermal, intramuscular, intranasal, buccal, sublingual or suppository administration, such as for topical administration.

86. HMGB1 binding polypeptide, fusion protein or conjugate according to any one of items 1-79 or a composition according to item 84 for use as a medicament, a diagnostic agent and/or a prognostic agent.

87. HMGB1 binding polypeptide, fusion protein, conjugate or composition for use according to item 86 as a medicament.

88. HMGB1 binding polypeptide, fusion protein, conjugate or composition for use according to item 86 as a diagnostic agent and/or a prognostic agent.

89. HMGB1 binding polypeptide, fusion protein, conjugate or composition for use as a medicament according to item 87, wherein said polypeptide, fusion protein, conjugate or composition modulates HMGB1 function in vivo.

90. HMGB1 binding polypeptide, fusion protein, conjugate or composition for use according to any one of items 86-89 in the treatment, prognosis or diagnosis of an HMGB1 related disorder.

91. HMGB1 binding polypeptide, fusion protein, conjugate or composition for use according to item 90, wherein said HMGB1 related disorder is selected from the group consisting of inflammatory diseases, respiratory diseases, autoimmune diseases, infectious diseases, trauma, cardiovascular disease, neurodegenerative diseases, metabolic disorders, liver injury and cancers.

92. HMGB1 binding polypeptide, fusion protein, conjugate or composition for use according to any one of items 90-91, wherein said HMGB1 related disorder is selected from the group consisting of arthritis (such as rheumatoid arthritis, collagen-induced arthritis, crystal-induced arthritis ankylosing spondylitis), atherosclerosis, hepatitis, inflammatory bowel disease, chronic inflammatory anemia, myositis, pancreatitis, pulmonary fibrosis, pulmonary inflammation, hepatic ischemia-reperfusion injury, drug-induced liver intoxication (such as acetaminophen/paracetamol intoxication), acute or chronic liver failure, nonalcoholic fatty liver disease, liver fibrosis, cirrhosis, hemolytic uremic syndrome, systemic lupus erythematosus, cutaneous lupus erythematosus, lupus nephritis, glomerulonephritis, juvenile idiopathic arthritis, antineutrophilic cytoplasmatic antibody (ANCA)-associated vasculitis, systemic vasculitis scleroderma, Sjogren syndrome, Behcet's disease, cancer (such as breast cancer, colorectal cancer, hepatocellular carcinoma, lung cancer, pancreatic cancer, renal cell carcinoma, melanoma and mesothelioma), ischemia/reperfusion, stroke, ischemic brain injury, chemical toxemia, traumatic brain injury, neuroinflammation, epileptogenesis, cognitive dysfunctions, atherosclerosis, gastric ulcer, hyperoxia, sepsis, endotoxemia, hemorrhagic shock, cerebrovascular disease, myocardial infarction, heart failure, Alzheimer's disease, multiple sclerosis, epilepsy, diabetes, obesity, transplant rejection, chronic kidney disease, sciatica and neuropathic pain.

93. HMGB1 binding polypeptide, fusion protein, conjugate or composition for use according to item 92, wherein said HMGB1 related disorder is selected from the group consisting of cerebrovascular diseases, chronic inflammatory anemia, acute liver failure, drug-induced liver intoxication, hemolytic uremic syndrome, systemic lupus erythematosus, mesothelioma, lung cancer, stroke, sepsis, sciatica and neuropathic pain, such as selected from the group consisting of drug-induced liver intoxication, sepsis, sciatica and neuropathic pain.

94. Method of treatment of an HMGB1 related disorder, comprising administering to a subject in need thereof an effective amount of an HMGB1 binding polypeptide, fusion protein or conjugate according to any one of items 1-79 or a composition according to item 84.

95. Method according to item 94, wherein said HMGB1 related disorder is selected from the group consisting of inflammatory diseases, respiratory diseases, autoimmune diseases, infectious diseases, trauma, cardiovascular disease, neurodegenerative diseases, metabolic disorders, liver injury and cancers.

96. Method according to item 95, wherein said HMGB1 related disorder is selected from the group consisting of arthritis (such as rheumatoid arthritis, collagen-induced arthritis, crystal-induced arthritis ankylosing spondylitis), atherosclerosis, hepatitis, inflammatory bowel disease, chronic inflammatory anemia, myositis, pancreatitis, pulmonary fibrosis, pulmonary inflammation, hepatic ischemia-reperfusion injury, drug-induced liver intoxication (such as acetaminophen/paracetamol intoxication), acute or chronic liver failure, nonalcoholic fatty liver disease, liver fibrosis, cirrhosis, hemolytic uremic syndrome, systemic lupus erythematosus, cutaneous lupus erythematosus, lupus nephritis, glomerulonephritis, juvenile idiopathic arthritis, antineutrophilic cytoplasmatic antibody (ANCA)-associated vasculitis, systemic vasculitis scleroderma, Sjogren syndrome, Behcet's disease, cancer (such as breast cancer, colorectal cancer, hepatocellular carcinoma, lung cancer, pancreatic cancer, renal cell carcinoma, melanoma and mesothelioma), ischemia/reperfusion, stroke, ischemic brain injury, chemical toxemia, traumatic brain injury, neuroinflammation, epileptogenesis, cognitive dysfunctions, atherosclerosis, gastric ulcer, hyperoxia, sepsis, endotoxemia, hemorrhagic shock, cerebrovascular disease, myocardial infarction, heart failure, Alzheimer's disease, multiple sclerosis, epilepsy, diabetes, obesity, transplant rejection, chronic kidney disease, sciatica and neuropathic pain.

97. Method according to item 96, wherein said HMGB1 related disorder is selected from the group consisting of cerebrovascular diseases, chronic inflammatory anemia, acute liver failure, drug-induced liver intoxication, hemolytic uremic syndrome, systemic lupus erythematosus, mesothelioma, lung cancer, stroke, sepsis, sciatica and neuropathic pain, such as selected from the group consisting of drug-induced liver intoxication, sepsis, sciatica and neuropathic pain.

98. Method of detecting HMGB1 in vitro, comprising providing a sample suspected to contain HMGB1, contacting said sample with an HMGB1 binding polypeptide, fusion protein or conjugate according to any one of items 1-79 or a composition according to item 84, and detecting the binding of the HMGB1 binding polypeptide, fusion protein, conjugate or composition to indicate the presence of HMGB1 in the sample.

99. Method for determining the presence of HMGB1 in a subject, comprising the steps of:
a) contacting the subject, or a sample isolated from the subject, with an HMGB1 binding polypeptide, fusion protein or conjugate according to any one of items 1-79 or a composition according to item 84, and
b) obtaining a value corresponding to the amount of the HMGB1 binding polypeptide, fusion protein, conjugate or composition that has bound in said subject or to said sample.

100. Method according to item 99, further comprising a step of comparing said value to a reference.

101. Method according to any one of items 99-100, wherein said subject is a mammalian subject, such as a human subject.

102. Method according to any one of items 99-101, wherein the method is performed in vivo.

1. A HMGB1 binding polypeptide, comprising an HMGB1 binding motif BM, which motif consists of an amino acid sequence selected from:
i)
(SEQ ID NO: 56)
EX2X3X4AX6X7EIX10 XI1LPNLX16X17X18Q
X20 X21AFIYX26LED
wherein, independently of each other,
X2 is selected from A, D, S and T;
X3 is selected from E, R and W;
X4 is selected from A, D and Q;
X6 is selected from F and M;
X7 is selected from E, H, W and Y;
X10 is selected from I and L;
X11 is selected from A and W;
X16 is selected from N and T;
X17 is selected from A, D, N and W;
X18 is selected from A, E, Q, R, S, T and Y;
X20 is selected from A and Q;
X21 is selected from K, L and R; and
X26 is selected from K and S;
and
ii) an amino acid sequence which has at least 93% identity to the sequence defined in i).

2. The HMGB1 binding polypeptide according to claim 1, wherein in sequence i)
X2 is selected from A, D, S and T;
X3 is selected from R and W;
X4 is D;
X6 is F;
X7 is selected from E, Y and W;
X10 is I;
X11 is W;
X16 is selected from N and T;
X17 is D;
X18 is selected from A, Q and R;
X20 is Q;
X21 is R; and
X26 is selected from K and S.

3. The HMGB1 binding polypeptide according to claim 1, wherein sequence i) corresponds to the sequence from position 8 to position 36 in a sequence selected from the group consisting of SEQ ID NO:1-14, 16 and 20-23.

4. The HMGB1 binding polypeptide according to claim 1, which binds to the A-box of HMGB1 such that the KD value of the interaction is at most 1×10⁻⁶M.

5. The HMGB1 binding polypeptide according to claim 1, wherein said HMGB1 binding motif forms part of a three-helix bundle protein domain.

6. The HMGB1 binding polypeptide according to claim 1, which comprises an amino acid sequence selected from:
xi)
(SEQ ID NO: 60)
AEAKYAK-[BM]-DPSQSSELLSEAKKLNDSQAPK;
wherein [BM] is an HMGB1 binding motif as defined in any one of claims 1-3; and
xii) an amino acid sequence which has at least 89% identity to the sequence defined in xi).

7. The HMGB1 binding polypeptide according to claim 6, wherein sequence xi) is selected from the group consisting of SEQ ID NO:27-28.

8. A fusion protein or conjugate comprising
a first moiety consisting of an HMGB1 binding polypeptide according to claim 1; and
a second moiety consisting of a polypeptide having a desired biological activity.

9. The fusion protein or conjugate according to claim 8, comprising at least two HMGB1 binding polypeptide monomer units as said first and second moiety, respectively, wherein said first HMGB1 binding polypeptide monomer unit has affinity for the A-box of HMGB1, and wherein said second HMGB1 binding polypeptide monomer unit has affinity for the B-box of HMGB1.

10. A composition comprising an HMGB1 binding polypeptide according to claim 1 and at least one pharmaceutically acceptable excipient or carrier.

11. (canceled)

12. (canceled)

13. (canceled)

14. (canceled)

15. A method of treatment of an HMGB1 related disorder, comprising administering to a subject in need thereof an effective amount of an HMGB1 binding polypeptide, according to claim 1.

16. A method for determining the presence of HMGB1 in a subject, comprising the steps of:
a) contacting the subject, or a sample isolated from the subject, with an HMGB1 binding polypeptide, according to claim 1, and
b) obtaining a value corresponding to the amount of the HMGB1 binding polypeptide, that has bound in said subject or to said sample.

17. The method of claim 15, wherein said HMGB1 related disorder is inflammatory diseases, respiratory diseases, autoimmune diseases, infectious diseases, trauma, cardiovascular disease, neurodegenerative diseases, metabolic disorders, liver injury, and cancer.

18. The HMGB1 binding polypeptide according to claim 1, which binds to the A-box of HMGB1 such that the KD value of the interaction is at most 5×10⁻⁷M.

19. The HMGB1 binding polypeptide according to claim 1, which binds to the A-box of HMGB1 such that the KD value of the interaction is at most 1×10⁻⁷M.

20. The HMGB1 binding polypeptide according to claim 1, which binds to the A-box of HMGB1 such that the KD value of the interaction is at most 5×10⁻⁸M

21. The HMGB1 binding polypeptide according to claim 1, which binds to the A-box of HMGB1 such that the KD value of the interaction is at most 1×10⁻⁸M.


SEQUENCE LISTING (132 sequences)

SEQ ID NO: 1 - protein, 58 aa, ARTIFICIAL SEQUENCE
  Engineered HMGB1 binding polypeptide
VDAKYAKEDWDAFWEIIWLPNLTDAQQRAFIYKLEDDPSQSSELLSEAKKLNDSQAPK

SEQ ID NO: 2 - protein, 58 aa, ARTIFICIAL SEQUENCE
  Engineered HMGB1 binding polypeptide
VDAKYAKEAEDAMHEILWLPNLTWEQQKAFIYKLEDDPSQSSELLSEAKKLNDSQAPK

SEQ ID NO: 3 - protein, 58 aa, ARTIFICIAL SEQUENCE
  Engineered HMGB1 binding polypeptide
VDAKYAKEAWQAMEEILWLPNLTNTQQLAFIYKLEDDPSQSSELLSEAKKLNDSQAPK

SEQ ID NO: 4 - protein, 58 aa, ARTIFICIAL SEQUENCE
  Engineered HMGB1 binding polypeptide
AEAKFAKEDWDAFYEIIWLPNLTDAQQRAFIYKLEDDPSQSSELLSEAKKLSESQAPK

SEQ ID NO: 5 - protein, 58 aa, ARTIFICIAL SEQUENCE
  Engineered HMGB1 binding polypeptide
AEAKFAKEARDAFYEIIWLPNLTDAQQRAFIYKLEDDPSQSSELLSEAKKLSESQAPK

SEQ ID NO: 6 - protein, 58 aa, ARTIFICIAL SEQUENCE
  Engineered HMGB1 binding polypeptide
AEAKFAKEAWDAFEEIIWLPNLTDAQQRAFIYKLEDDPSQSSELLSEAKKLSESQAPK

SEQ ID NO: 7 - protein, 58 aa, ARTIFICIAL SEQUENCE
  Engineered HMGB1 binding polypeptide
AEAKFAKEAWDAFYEIIWLPNLTDAQQRAFIYKLEDDPSQSSELLSEAKKLSESQAPK

SEQ ID NO: 8 - protein, 58 aa, ARTIFICIAL SEQUENCE
  Engineered HMGB1 binding polypeptide
AEAKFAKEAWDAFYEIIWLPNLTDRQQRAFIYKLEDDPSQSSELLSEAKKLSESQAPK

SEQ ID NO: 9 - protein, 58 aa, ARTIFICIAL SEQUENCE
  Engineered HMGB1 binding polypeptide
AEAKFAKETWDAFYEIIWLPNLTDAQQRAFIYKLEDDPSQSSELLSEAKKLSESQAPK

SEQ ID NO: 10 - protein, 58 aa, ARTIFICIAL SEQUENCE
  Engineered HMGB1 binding polypeptide
AEAKFAKESWDAFYEIIWLPNLTDQQQRAFIYKLEDDPSQSSELLSEAKKLSESQAPK

SEQ ID NO: 11 - protein, 58 aa, ARTIFICIAL SEQUENCE
  Engineered HMGB1 binding polypeptide
AEAKFAKEDWDAFYEIIWLPNLTDEQQRAFIYKLEDDPSQSSELLSEAKKLSESQAPK

SEQ ID NO: 12 - protein, 58 aa, ARTIFICIAL SEQUENCE
  Engineered HMGB1 binding polypeptide
AEAKFAKEDWDAFWEIIWLPNLTDYQQRAFIYKLEDDPSQSSELLSEAKKLSESQAPK

SEQ ID NO: 13 - protein, 58 aa, ARTIFICIAL SEQUENCE
  Engineered HMGB1 binding polypeptide
AEAKFAKEAWDAFYEIIWLPNLTDYQQRAFIYKLEDDPSQSSELLSEAKKLSESQAPK

SEQ ID NO: 14 - protein, 58 aa, ARTIFICIAL SEQUENCE
  Engineered HMGB1 binding polypeptide
VDAKYAKEAWDAFWEIIWLPNLTDAQQRAFIYKLEDDPSQSSELLSEAKKLNDSQAPK

SEQ ID NO: 15 - protein, 58 aa, ARTIFICIAL SEQUENCE
  Engineered HMGB1 binding polypeptide
VDAKYAKEDADAFWEIIWLPNLTDAQQRAFIYKLEDDPSQSSELLSEAKKLNDSQAPK

SEQ ID NO: 16 - protein, 58 aa, ARTIFICIAL SEQUENCE
  Engineered HMGB1 binding polypeptide
VDAKYAKEDWAAFWEIIWLPNLTDAQQRAFIYKLEDDPSQSSELLSEAKKLNDSQAPK

SEQ ID NO: 17 - protein, 58 aa, ARTIFICIAL SEQUENCE
  Engineered HMGB1 binding polypeptide
VDAKYAKEDWDAAWEIIWLPNLTDAQQRAFIYKLEDDPSQSSELLSEAKKLNDSQAPK

SEQ ID NO: 18 - protein, 58 aa, ARTIFICIAL SEQUENCE
  Engineered HMGB1 binding polypeptide
VDAKYAKEDWDAFAEIIWLPNLTDAQQRAFIYKLEDDPSQSSELLSEAKKLNDSQAPK

SEQ ID NO: 19 - protein, 58 aa, ARTIFICIAL SEQUENCE
  Engineered HMGB1 binding polypeptide
VDAKYAKEDWDAFWEIAWLPNLTDAQQRAFIYKLEDDPSQSSELLSEAKKLNDSQAPK

SEQ ID NO: 20 - protein, 58 aa, ARTIFICIAL SEQUENCE
  Engineered HMGB1 binding polypeptide
VDAKYAKEDWDAFWEIIALPNLTDAQQRAFIYKLEDDPSQSSELLSEAKKLNDSQAPK

SEQ ID NO: 21 - protein, 58 aa, ARTIFICIAL SEQUENCE
  Engineered HMGB1 binding polypeptide
VDAKYAKEDWDAFWEIIWLPNLTAAQQRAFIYKLEDDPSQSSELLSEAKKLNDSQAPK

SEQ ID NO: 22 - protein, 58 aa, ARTIFICIAL SEQUENCE
  Engineered HMGB1 binding polypeptide
VDAKYAKEDWDAFWEIIWLPNLTDSQQRAFIYKLEDDPSQSSELLSEAKKLNDSQAPK

SEQ ID NO: 23 - protein, 58 aa, ARTIFICIAL SEQUENCE
  Engineered HMGB1 binding polypeptide
VDAKYAKEDWDAFWEIIWLPNLTDAQARAFIYKLEDDPSQSSELLSEAKKLNDSQAPK

SEQ ID NO: 24 - protein, 58 aa, ARTIFICIAL SEQUENCE
  Engineered HMGB1 binding polypeptide
VDAKYAKEDWDAFWEIIWLPNLTDAQQAAFIYKLEDDPSQSSELLSEAKKLNDSQAPK

SEQ ID NO: 25 - protein, 58 aa, ARTIFICIAL SEQUENCE
  Engineered HMGB1 binding polypeptide
VDAKYAKEDWDAFWEIIWLPNLTDAQQRAFIAKLEDDPSQSSELLSEAKKLNDSQAPK

SEQ ID NO: 26 - protein, 58 aa, ARTIFICIAL SEQUENCE
  Engineered HMGB1 binding polypeptide
VDAKYAKEDWDAFWEIIWLPNLTDAQQRAFIYKLADDPSQSSELLSEAKKLNDSQAPK

SEQ ID NO: 27 - protein, 58 aa, ARTIFICIAL SEQUENCE
  Engineered HMGB1 binding polypeptide
AEAKYAKEDWDAFWEIIWLPNLTDAQQRAFIYKLEDDPSQSSELLSEAKKLNDSQAPK

SEQ ID NO: 28 - protein, 58 aa, ARTIFICIAL SEQUENCE
  Engineered HMGB1 binding polypeptide
AEAKYAKEAWQAMEEILWLPNLTNTQQLAFIYKLEDDPSQSSELLSEAKKLNDSQAPK

SEQ ID NO: 29 - protein, 58 aa, ARTIFICIAL SEQUENCE
  Engineered HMGB1 binding polypeptide
AEAKFAKEDWDAFWEIIWLPNLTDAQQRAFIYKLEDDPSQSSELLSEAKKLSESQAPK

SEQ ID NO: 30 - protein, 58 aa, ARTIFICIAL SEQUENCE
  Engineered HMGB1 binding polypeptide
VDAKYAKEAWSAEQEIWDLPNLTLQQFQAFIMKLLDDPSQSSELLSEAKKLNDSQAPK

SEQ ID NO: 31 - protein, 58 aa, ARTIFICIAL SEQUENCE
  Engineered HMGB1 binding polypeptide
VDAKYAKEAWLAEQEIWQLPNLTEAQFQAFILKLIDDPSQSSELLSEAKKLNDSQAPK

SEQ ID NO: 32 - protein, 58 aa, ARTIFICIAL SEQUENCE
  Engineered HMGB1 binding polypeptide
AEAKYAKEAWLAEQEIWQLPNLTEAQFQAFILKLIDDPSQSSELLSEAKKLNDSQAPK

SEQ ID NO: 33 - protein, 58 aa, ARTIFICIAL SEQUENCE
  Engineered negative control polypeptide
AEAKYAKELGWATWEIFNLPNLTGVQVKAFIDKLRDDPSQSSELLSEAKKLNDSQAPK

SEQ ID NO: 34 - protein, 46 aa, ARTIFICIAL SEQUENCE
  Engineered albumin binding polypeptide
LAEAKEAANAELDSYGVSDFYKRLIDKAKTVEGVEALKDAILAALP

SEQ ID NO: 35 - protein, 108 aa, ARTIFICIAL SEQUENCE
  Engineered HMGB1 binding polypeptide
AEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 36 - protein, 108 aa, ARTIFICIAL SEQUENCE
  Engineered HMGB1 binding polypeptide
AEAKYAKEAWQAMEEILWLPNLTNTQQLAFIYKLEDDPSQSSELLSEAKKLNDSQAPKASGSLAEAKEAANAELDSYGVSDFYKRLIDKAKTVEGVEALKDAILAALP

SEQ ID NO: 37 - protein, 108 aa, ARTIFICIAL SEQUENCE
  Engineered HMGB1 binding polypeptide
AEAKYAKEAWLAEQEIWQLPNLTEAQFQAFILKLIDDPSQSSELLSEAKKLNDSQAPKASGSLAEAKEAANAELDSYGVSDFYKRLIDKAKTVEGVEALKDAILAALP

SEQ ID NO: 38 - protein, 186 aa, ARTIFICIAL SEQUENCE
  Engineered HMGB1 binding polypeptide
VDAKYAKEDWDAFWEIIWLPNLTDAQQRAFIYKLEDDPSQSSELLSEAKKLNDSQAPKGAPGGGGSGGGGSGGGGSTSVDAKYAKEAWLAEQEIWQLPNLTEAQFQAFILKLIDDPSQSSELLSEAKKLNDSQAPKASGSLAEAKEAANAELDSYGVSDFYKRLIDKAKTVEGVEALKDAILAALP

SEQ ID NO: 39 - protein, 176 aa, ARTIFICIAL SEQUENCE
  Engineered HMGB1 binding polypeptide
VDAKYAKEDWDAFWEIIWLPNLTDAQQRAFIYKLEDDPSQSSELLSEAKKLNDSQAPKGAPGGGGSTSVDAKYAKEAWLAEQEIWQLPNLTEAQFQAFILKLIDDPSQSSELLSEAKKLNDSQAPKASGSLAEAKEAANAELDSYGVSDFYKRLIDKAKTVEGVEALKDAILAALP

SEQ ID NO: 40 - protein, 186 aa, ARTIFICIAL SEQUENCE
  Engineered HMGB1 binding polypeptide
VDAKYAKEAWLAEQEIWQLPNLTEAQFQAFILKLIDDPSQSSELLSEAKKLNDSQAPKGAPGGGGSGGGGSGGGGSTSVDAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 41 - protein, 176 aa, ARTIFICIAL SEQUENCE
  Engineered HMGB1 binding polypeptide
VDAKYAKEAWLAEQEIWQLPNLTEAQFQAFILKLIDDPSQSSELLSEAKKLNDSQAPKGAPGGGGSTSVDAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 42 - protein, 173 aa, ARTIFICIAL SEQUENCE
  Engineered HMGB1 binding polypeptide
VDAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALPGTGGGGSVDAKYAKEAWLAEQEIWQLPNLTEAQFQAFILKLIDDPSQSSELLSEAKKLNDSQAPK

SEQ ID NO: 43 - protein, 186 aa, ARTIFICIAL SEQUENCE
  Engineered HMGB1 binding polypeptide
AEAKYAKEDWDAFWEIIWLPNLTDAQQRAFIYKLEDDPSQSSELLSEAKKLNDSQAPKGAPGGGGSGGGGSGGGGSTSAEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 44 - protein, 176 aa, ARTIFICIAL SEQUENCE
  Engineered HMGB1 binding polypeptide
AEAKYAKEDWDAFWEIIWLPNLTDAQQRAFIYKLEDDPSQSSELLSEAKKLNDSQAPKGAPGGGGSTSAEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 45 - protein, 173 aa, ARTIFICIAL SEQUENCE
  Engineered HMGB1 binding polypeptide
AEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALPGTGGGGSAEAKYAKEDWDAFWEIIWLPNLTDAQQRAFIYKLEDDPSQSSELLSEAKKLNDSQAPK

SEQ ID NO: 46 - protein, 176 aa, ARTIFICIAL SEQUENCE
  Engineered HMGB1 binding polypeptide
AEAKYAKEDWDAFWEIIWLPNLTDAQQRAFIYKLEDDPSQSSELLSEAKKLNDSQAPKGAPGGGGSTSAEAKYAKEAWLAEQEIWQLPNLTEAQFQAFILKLIDDPSQSSELLSEAKKLNDSQAPKASGSLAEAKEAANAELDSYGVSDFYKRLIDKAKTVEGVEALKDAILAALP

SEQ ID NO: 47 - protein, 186 aa, ARTIFICIAL SEQUENCE
  Engineered HMGB1 binding polypeptide
AEAKYAKEAWLAEQEIWQLPNLTEAQFQAFILKLIDDPSQSSELLSEAKKLNDSQAPKGAPGGGGSGGGGSGGGGSTSAEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 48 - protein, 176 aa, ARTIFICIAL SEQUENCE
  Engineered HMGB1 binding polypeptide
AEAKYAKEAWLAEQEIWQLPNLTEAQFQAFILKLIDDPSQSSELLSEAKKLNDSQAPKGAPGGGGSTSAEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALP

SEQ ID NO: 49 - protein, 176 aa, ARTIFICIAL SEQUENCE
  Engineered negative control polypeptide
AEAKYAKELGWATWEIFNLPNLTGVQVKAFIDKLRDDPSQSSELLSEAKKLNDSQAPKGAPGGGGSTSAEAKYAKELGWATWEIFNLPNLTGVQVKAFIDKLRDDPSQSSELLSEAKKLNDSQAPKASGSLAEAKEAANAELDSYGVSDFYKRLIDKAKTVEGVEALKDAILAALP

SEQ ID NO: 50 - protein, 215 aa, RATTUS NORVEGICUS
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 51 - protein, 89 aa, HOMO SAPIENS
MGKGDPKKPRGKMSSYAFFVQTCREEHKKKHPDASVNFSEFSKKCSERWKTMSAKEKGKFEDMAKADKARYEREMKTYIPPKGETKKKF

SEQ ID NO: 52 - protein, 324 aa, HOMO SAPIENS
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPGILMGKGDPKKPRGKMSSYAFFVQTCREEHKKKHPDASVNFSEFSKKCSERWKTMSAKEKGKFEDMAKADKARYEREMKTYIPPKGETKKKF

SEQ ID NO: 53 - protein, 311 aa, HOMO SAPIENS
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPEFALKFKDPNAPKRPPSAFFLFCSEYRPKIKGEHPGLSIGDVAKKLGEMWNNTAADDKQPYEKKAAKLKEKYEKDIAAY

SEQ ID NO: 54 - protein, 58 aa, ARTIFICIAL SEQUENCE
  Engineered negative control polypeptide
VDAKYAKELGWATWEIFNLPNLTGVQVKAFIDKLRDDPSQSSELLSEAKKLNDSQAPK

SEQ ID NO: 55 - protein, 178 aa, ARTIFICIAL SEQUENCE
  Engineered HMGB1 binding polypeptide
AEAKYAKEAWLAEQEIWQLPNLTEAQFQAFILKLIDDPSQSSELLSEAKKLNDSQAPKGAPGGGGSTSAEAKYAKEDWDAFWEIIWLPNLTDAQQRAFIYKLEDDPSQSSELLSEAKKLNDSQAPKASGSLAEAKEAANAELDSYGVSDFYKRLIDKAKTVEGVEALKDAILAALPGC

SEQ ID NO: 56 - protein, 29 aa, ARTIFICIAL SEQUENCE
  Engineered HMGB1 binding polypeptide
  VARIANT (2)..(2): A, D, S or T
  VARIANT (3)..(3): E, R or W
  VARIANT (4)..(4): A, D or Q
  VARIANT (6)..(6): F or M
  VARIANT (7)..(7): E, H, W or Y
  VARIANT (10)..(10): I or L
  VARIANT (11)..(11): A or W
  VARIANT (16)..(16): N or T
  VARIANT (17)..(17): A, D, N or W
  VARIANT (18)..(18): A, E, Q, R, S, T or Y
  VARIANT (20)..(20): A or Q
  VARIANT (21)..(21): K, L or R
  VARIANT (26)..(26): K or S
EXXXAXXEIXXLPNLXXXQXXAFIYXLED

SEQ ID NO: 57 - protein, 29 aa, ARTIFICIAL SEQUENCE
  Engineered HMGB1 binding polypeptide
  VARIANT (4)..(4): A, D, E, F, G, H, I, K, L, M, N, Q, R, S, T, V, W or Y
  VARIANT (11)..(11): A, D, E, F, G, H, I, K, L, M, N, Q, R, S, T, V, W or Y
  VARIANT (16)..(16): N or T
  VARIANT (17)..(18): A, D, E, F, G, H, I, K, L, M, N, Q, R, S, T, V, W or Y
  VARIANT (25)..(25): A, D, E, F, G, H, I, K, L, M, N, Q, R, S, T, V, W or Y
  VARIANT (26)..(26): K or S
  VARIANT (28)..(28): A, D, E, F, G, H, I, K, L, M, N, Q, R, S, T, V, W or Y
EAWXAEQEIWXLPNLXXXQFQAFIXXLXD

SEQ ID NO: 58 - protein, 49 aa, ARTIFICIAL SEQUENCE
  Engineered HMGB1 binding polypeptide
  misc_feature (3)..(5): Xaa can be any naturally occurring amino acid
  misc_feature (7)..(8): Xaa can be any naturally occurring amino acid
  misc_feature (11)..(12): Xaa can be any naturally occurring amino acid
  misc_feature (17)..(19): Xaa can be any naturally occurring amino acid
  misc_feature (21)..(22): Xaa can be any naturally occurring amino acid
  misc_feature (26)..(27): Xaa can be any naturally occurring amino acid
  misc_feature (29)..(29): Xaa can be any naturally occurring amino acid
  VARIANT (36)..(36): A or S
  VARIANT (37)..(37): N or E
  VARIANT (40)..(40): A, S or C
  VARIANT (46)..(46): E, N or S
  VARIANT (47)..(47): D, E or S
  VARIANT (48)..(48): A or S
KEXXXAXXEIXXLPNLXXXQXXAFIXXLXDDPSQSXXLLXEAKKLXXXQ

SEQ ID NO: 59 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLSEAKKLNDSQAPK

SEQ ID NO: 60 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLNDSQAPK

SEQ ID NO: 61 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKFAKEXXXAXXEIXXLPNLXXXQXXAFIXXLXDDPSQSSELLSEAKKLSESQAPK

SEQ ID NO: 62 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKFAKEXXXAXXEIXXLPNLXXXQXXAFIXXLXDDPSQSSELLSEAKKLNDSQAPK

SEQ ID NO: 63 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
ADNNFNKEXXXAXXEIXXLPNLXXXQXXAFIXXLXDDPSQSANLLSEAKKLNESQAPK

SEQ ID NO: 64 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
ADNKFNKEXXXAXXEIXXLPNLXXXQXXAFIXXLXDDPSQSANLLAEAKKLNDAQAPK

SEQ ID NO: 65 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
ADNKFNKEXXXAXXEIXXLPNLXXXQXXAFIXXLXDDPSVSKEILAEAKKLNDAQAPK

SEQ ID NO: 66 - protein, 61 aa, ARTIFICIAL SEQUENCE
  Engineered HMGB1 binding polypeptide
  misc_feature (12)..(14): Xaa can be any naturally occurring amino acid
  misc_feature (16)..(17): Xaa can be any naturally occurring amino acid
  misc_feature (20)..(21): Xaa can be any naturally occurring amino acid
  misc_feature (26)..(28): Xaa can be any naturally occurring amino acid
  misc_feature (30)..(31): Xaa can be any naturally occurring amino acid
  misc_feature (35)..(36): Xaa can be any naturally occurring amino acid
  misc_feature (38)..(38): Xaa can be any naturally occurring amino acid
ADAQQNNFNKEXXXAXXEIXXLPNLXXXQXXAFIXXLXDDPSQSTNVLGEAKKLNESQAPK

SEQ ID NO: 67 - protein, 56 aa, ARTIFICIAL SEQUENCE
  Engineered HMGB1 binding polypeptide
  misc_feature (7)..(9): Xaa can be any naturally occurring amino acid
  misc_feature (11)..(12): Xaa can be any naturally occurring amino acid
  misc_feature (15)..(16): Xaa can be any naturally occurring amino acid
  misc_feature (21)..(23): Xaa can be any naturally occurring amino acid
  misc_feature (25)..(26): Xaa can be any naturally occurring amino acid
  misc_feature (30)..(31): Xaa can be any naturally occurring amino acid
  misc_feature (33)..(33): Xaa can be any naturally occurring amino acid
AQHDEEXXXAXXEIXXLPNLXXXQXXAFIXXLXDDPSQSANVLGEAQKLNDSQAPK

SEQ ID NO: 68 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDNKFNKEXXXAXXEIXXLPNLXXXQXXAFIXXLXDDPSQSANLLAEAKKLNDAQAPK

SEQ ID NO: 69 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSESSELLSEAKKLNKSQAPK

SEQ ID NO: 70 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLNDAQAPK

SEQ ID NO: 71 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLNDSQAPK

SEQ ID NO: 72 - protein, 58 aa, ARTIFICIAL SEQUENCE; identical to SEQ ID NO: 60
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid

SEQ ID NO: 73 - protein, 57 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLNDSQAP

SEQ ID NO: 74 - protein, 58 aa, ARTIFICIAL SEQUENCE; identical to SEQ ID NO: 62
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid

SEQ ID NO: 75 - protein, 57 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKFAKEXXXAXXEIXXLPNLXXXQXXAFIXXLXDDPSQSSELLSEAKKLNDSQAP

SEQ ID NO: 76 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLAEAKKLNDAQAPK

SEQ ID NO: 77 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLSESQAPK

SEQ ID NO: 78 - protein, 57 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLSESQAP

SEQ ID NO: 79 - protein, 58 aa, ARTIFICIAL SEQUENCE; identical to SEQ ID NO: 61
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid

SEQ ID NO: 80 - protein, 57 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKFAKEXXXAXXEIXXLPNLXXXQXXAFIXXLXDDPSQSSELLSEAKKLSESQAP

SEQ ID NO: 81 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLAEAKKLSEAQAPK

SEQ ID NO: 82 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDQPEQSSELLSEAKKLSESQAPK

SEQ ID NO: 83 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLESSQAPK

SEQ ID NO: 84 - protein, 57 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLESSQAP

SEQ ID NO: 85 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLAEAKKLESAQAPK

SEQ ID NO: 86 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDQPEQSSELLSEAKKLESSQAPK

SEQ ID NO: 87 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLSDSQAPK

SEQ ID NO: 88 - protein, 57 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLSEAKKLSDSQAP

SEQ ID NO: 89 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLAEAKKLSDSQAPK

SEQ ID NO: 90 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLAEAKKLSDAQAPK

SEQ ID NO: 91 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDQPEQSSELLSEAKKLSDSQAPK

SEQ ID NO: 92 - protein, 58 aa, ARTIFICIAL SEQUENCE; identical to SEQ ID NO: 59
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid

SEQ ID NO: 93 - protein, 58 aa, ARTIFICIAL SEQUENCE; identical to SEQ ID NO: 70
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid

SEQ ID NO: 94 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLSEAKKLSESQAPK

SEQ ID NO: 95 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLSEAQAPK

SEQ ID NO: 96 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDQPEQSSELLSEAKKLSESQAPK

SEQ ID NO: 97 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLSEAKKLESSQAPK

SEQ ID NO: 98 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLESAQAPK

SEQ ID NO: 99 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDQPEQSSELLSEAKKLESSQAPK

SEQ ID NO: 100 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLSEAKKLSDSQAPK

SEQ ID NO: 101 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLSDSQAPK

SEQ ID NO: 102 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLSDAQAPK

SEQ ID NO: 103 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDQPEQSSELLSEAKKLSDSQAPK

SEQ ID NO: 104 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
VDAKYAKEXXXAXXEIXXLPNLXXXQXXAFIXXLXDDPSQSSELLAEAKKLNKAQAPK

SEQ ID NO: 105 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
AEAKYAKEXXXAXXEIXXLPNLXXXQXXAFIXXLXDDPSQSSELLAEAKKLNKAQAPK

SEQ ID NO: 106 - protein, 58 aa, ARTIFICIAL SEQUENCE
  Engineered HMGB1 binding polypeptide
  misc_feature (9)..(11): Xaa can be any naturally occurring amino acid
  misc_feature (13)..(14): Xaa can be any naturally occurring amino acid
  misc_feature (17)..(18): Xaa can be any naturally occurring amino acid
  misc_feature (23)..(25): Xaa can be any naturally occurring amino acid
  misc_feature (27)..(28): Xaa can be any naturally occurring amino acid
  misc_feature (32)..(33): Xaa can be any naturally occurring amino acid
  misc_feature (35)..(35): Xaa can be any naturally occurring amino acid
ADAKYAKEXXXAXXEIXXLPNLXXXQXXAFIXXLXDDPSQSSELLSEAKKLNDSQAPK

SEQ ID NO: 107 - protein, 8 aa, ARTIFICIAL SEQUENCE
  Engineered amino acid linker
GAPGGGGS

SEQ ID NO: 108 - protein, 16 aa, ARTIFICIAL SEQUENCE
  Engineered amino acid linker
GGGGSLVPRGSGGGGS

SEQ ID NO: 109 - protein, 6 aa, ARTIFICIAL SEQUENCE
  Engineered amino acid linker
GSGSGS

SEQ ID NO: 110 - protein, 8 aa, ARTIFICIAL SEQUENCE
  Engineered amino acid linker
GSGSGSGS

SEQ ID NO: 111 - protein, 16 aa, ARTIFICIAL SEQUENCE
  Engineered amino acid linker
GSGSGSGSGSGSGSGS

SEQ ID NO: 112 - protein, 11 aa, ARTIFICIAL SEQUENCE
  Engineered amino acid linker
GGSGGHMGSGG

SEQ ID NO: 113 - protein, 11 aa, ARTIFICIAL SEQUENCE
  Engineered amino acid linker
GGSGGSGGSGG

SEQ ID NO: 114 - protein, 5 aa, ARTIFICIAL SEQUENCE
  Engineered amino acid linker
GGSGG

SEQ ID NO: 115 - protein, 8 aa, ARTIFICIAL SEQUENCE
  Engineered amino acid linker
GGSGGGGG

SEQ ID NO: 116 - protein, 18 aa, ARTIFICIAL SEQUENCE
  Engineered amino acid linker
GGGSEGGGSEGGGSEGGG

SEQ ID NO: 117 - protein, 8 aa, ARTIFICIAL SEQUENCE
  Engineered amino acid linker
AAGAATAA

SEQ ID NO: 118 - protein, 5 aa, ARTIFICIAL SEQUENCE
  Engineered amino acid linker
GGGGG

SEQ ID NO: 119 - protein, 5 aa, ARTIFICIAL SEQUENCE
  Engineered amino acid linker
GGSSG

SEQ ID NO: 120 - protein, 11 aa, ARTIFICIAL SEQUENCE
  Engineered amino acid linker
GSGGGTGGGSG

SEQ ID NO: 121 - protein, 11 aa, ARTIFICIAL SEQUENCE
  Engineered amino acid linker
GSGGGTGGGSG

SEQ ID NO: 122 - protein, 12 aa, ARTIFICIAL SEQUENCE
  Engineered amino acid linker
GSGSGSGSGGSG

SEQ ID NO: 123 - protein, 14 aa, ARTIFICIAL SEQUENCE
  Engineered amino acid linker
GSGGSGGSGGSGGS

SEQ ID NO: 124 - protein, 14 aa, ARTIFICIAL SEQUENCE
  Engineered amino acid linker
GSGGSGSGGSGGSG

SEQ ID NO: 125 - protein, 5 aa, ARTIFICIAL SEQUENCE
  Engineered amino acid linker
SSSSG

SEQ ID NO: 126 - protein, 15 aa, ARTIFICIAL SEQUENCE
  Engineered amino acid linker
SSSSGSSSSGSSSSG

SEQ ID NO: 127 - protein, 20 aa, ARTIFICIAL SEQUENCE
  Engineered amino acid linker
SSSSGSSSSGSSSSGSSSSG

SEQ ID NO: 128 - protein, 5 aa, ARTIFICIAL SEQUENCE
  Engineered amino acid linker
GGGGS

SEQ ID NO: 129 - protein, 15 aa, ARTIFICIAL SEQUENCE
  Engineered amino acid linker
GGGGSGGGGSGGGGS

SEQ ID NO: 130 - protein, 4 aa, ARTIFICIAL SEQUENCE
  Engineered amino acid linker
ASGS

SEQ ID NO: 131 - protein, 120 aa, ARTIFICIAL SEQUENCE
  Engineered fusion polypeptide
  misc_feature (17)..(19): Xaa can be any naturally occurring amino acid
  misc_feature (21)..(22): Xaa can be any naturally occurring amino acid
  misc_feature (25)..(26): Xaa can be any naturally occurring amino acid
  misc_feature (31)..(33): Xaa can be any naturally occurring amino acid
  misc_feature (35)..(36): Xaa can be any naturally occurring amino acid
  misc_feature (40)..(41): Xaa can be any naturally occurring amino acid
  misc_feature (43)..(43): Xaa can be any naturally occurring amino acid
AQHDEALEAEAKFAKEXXXAXXEIXXLPNLXXXQXXAFIXXLXDDPSQSSELLSEAKKLSESQAPKVDYVLAEAKEAANAELDSYGVSDFYKRLIDKAKTVEGVEALKDAILAALPYVPG

SEQ ID NO: 132 - protein, 62 aa, ARTIFICIAL SEQUENCE
  Engineered control polypeptide
AQHDEALEVDYVLAEAKEAANAELDSYGVSDFYKRLIDKAKTVEGVEALKDAILAALPYVPG